(19) United States
(12) Patent Application Publication
(10) Pub. No.: US 2026/0250235 A1
(43) Pub. Date: Aug. 27, 2026

(54) FLUORINATED ESTERS, METHODS OF SYNTHESIZING THE SAME, AND USES OF THE SAME

(71) Applicant: Solstice Advanced Materials US, Inc., Morris Plains, NJ (US)

(72) Inventors: Xanath Ispizua Rodriguez, East Amherst, NY (US); David Charles Bock, Orchard Park, NY (US); Robert Joseph Stewart, East Aurora, NY (US); Sergey Nicholas Maximoff, Glenview, NY (US); Pramod Warrier, Buffalo, NY (US)

(21) Appl. No.: 19/549,301

(22) Filed: Feb. 25, 2026

Related U.S. Application Data
(60) Provisional application No. 63/762,925, filed on Feb. 25, 2025.
(60) Provisional application No. 63/843,733, filed on Jul. 14, 2025.
(60) Provisional application No. 63/899,624, filed on Oct. 15, 2025.
(60) Provisional application No. 63/988,914, filed on Feb. 23, 2026.

(51) Int. Cl. C07C 69/63; C07C 67/04; H01M 10/0567; H01M 10/42

(52) U.S. Cl. CPC C07C 69/63; C07C 67/04; H01M 10/0567; H01M 10/4235; H01M 2300/0025

(57) ABSTRACT

A fluorinated ester selected from 1,2-difluoroethyl 3,3,3-trifluoropropanoate, 1,1-difluoroethyl 3,3,3-trifluoropropanoate, or 1,1-difluoroethyl 2,2-difluoroacetate, methods of synthesizing the same, and uses of the same.

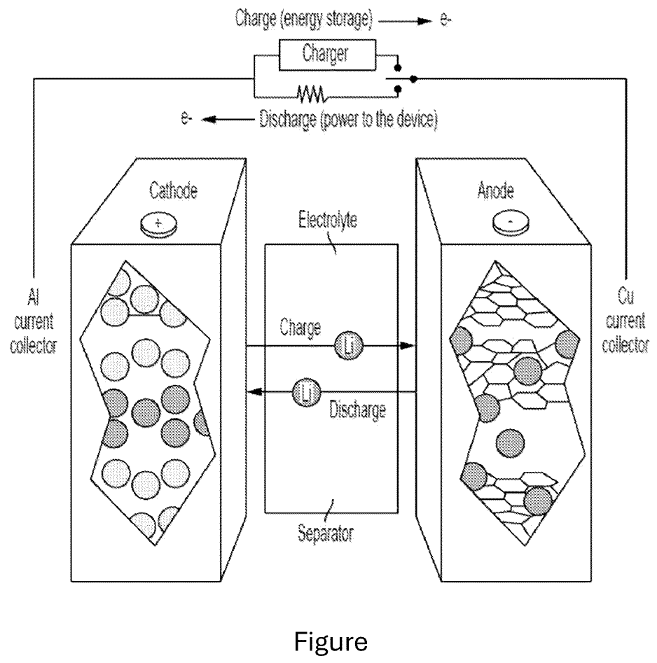

### CROSS REFERENCE TO RELATED APPLICATIONS

The present application claims the benefit of priority to United States Application Nos. 63/762,925, filed Feb. 25, 2025, 63/843,733, filed Jul. 14, 2025, 63/899,624, filed Oct. 15, 2025, and 63/988,914, filed Feb. 23, 2026, the contents of which in their entirety are herein incorporated by reference.

### FIELD

The present disclosure is directed to fluorinated esters, methods of synthesizing the same, and uses of the same, for example, incorporation into electrolytes for use in electrochemical energy storage devices.

### BACKGROUND

Electrochemical energy storage devices (also referred to as electrochemical cells or batteries) providing high charging voltage, wide operating temperature range, fast charge, and resistance to mechanical and thermal abuse, while exhibiting long cycle and calendar life are desired. Also desired are electrolytes for use in batteries that provide improved performance.

Further desired are compounds for inclusion in electrolytes for use in batteries that provide desirable characteristics to the batteries. Provided are compounds that provide desirable characteristics to electrolytes and batteries including such electrolytes.

### SUMMARY

The present disclosure provides a fluorinated ester selected from 1,2-difluoroethyl 3,3,3-trifluoropropanoate, 1,1-difluoroethyl 3,3,3-trifluoropropanoate, or 1,1-difluoroethyl 2,2-difluoroacetate. 1,2-difluoroethyl 3,3,3-trifluoropropanoate is also known as 1,2-difluoroethyl 3,3,3-trifluoropropionate and 1,1-difluoroethyl 3,3,3-trifluoropropanoate is also known as 1,1-difluoroethyl 3,3,3-trifluoropropionate.

The present disclosure is based on the discovery that fluorinated esters 1,2-difluoroethyl 3,3,3-trifluoropropanoate, 1,1-difluoroethyl 3,3,3-trifluoropropanoate, and 1,1-difluoroethyl 2,2-difluoroacetate provide beneficial properties to electrolytes and batteries including such electrolytes.

The present disclosure provides a method of synthesizing a fluorinated ester including (a) providing a carboxylic acid or carboxylic acid derivative; and (b) reacting the carboxylic acid or carboxylic acid derivative with a fluorinated olefin to synthesize the fluorinated ester. Exemplary fluorinated olefins include (E)-1,2-dichloro-1,2-difluoroethene, (Z)-1,2-dichloro-1,2-difluoroethene, 1,1-difluoroethene, and 1,1-dichloro-2,2-difluoroethene.

The present disclosure provides a method of synthesizing a fluorinated ester including (a) providing a fluorinated carboxylic acid or fluorinated carboxylic acid derivative; and (b) reacting the fluorinated carboxylic acid or fluorinated carboxylic acid derivative with a fluorinated olefin to synthesize the fluorinated ester.

The present disclosure provides 1,2-difluoroethyl 3,3,3-trifluoropropanoate and electrolytes including same.

The present disclosure provides 1,1-difluoroethyl 3,3,3-trifluoropropanoate and electrolytes including same.

The present disclosure provides 1,1-difluoroethyl 2,2-difluoroacetate and electrolytes including same.

The present disclosure provides a method of synthesizing 1,2-difluoroethyl 3,3,3-trifluoropropanoate, 1,1-difluoroethyl 3,3,3-trifluoropropanoate, or 1,1-difluoroethyl 2,2-difluoroacetate including (a) providing a fluorinated carboxylic acid or fluorinated carboxylic acid derivative; (b) reacting the fluorinated carboxylic acid or fluorinated carboxylic acid derivative with a fluorinated olefin to synthesize a fluorinated ester intermediate; and (c) selectively dehalogenating the fluorinated ester intermediate to synthesize the fluorinated ester.

The present disclosure provides a method of synthesizing 1,2-difluoroethyl 3,3,3-trifluoropropanoate, 1,1-difluoroethyl 3,3,3-trifluoropropanoate, or 1,1-difluoroethyl 2,2-difluoroacetate including providing a fluorinated carboxylate salt; reacting the fluorinated carboxylate salt with chlorine monofluoride to form a fluorinated hypochlorous anhydride; reacting the fluorinated hypochlorous anhydride with a fluorinated olefin to synthesize a fluorinated ester intermediate; and selectively dehalogenating the fluorinated ester intermediate to synthesize the fluorinated ester. The fluorinated carboxylate salt can be a fluorinated sodium carboxylate salt.

The present disclosure provides an electrolyte including 1,2-difluoroethyl 3,3,3-trifluoropropanoate, 1,1-difluoroethyl 3,3,3-trifluoropropanoate, 1,1-difluoroethyl 2,2-difluoroacetate, or a combination thereof; and lithium bis(trifluoromethanesulfonyl)imide, lithium bis(fluorosulfonyl)imide, lithium bis(oxalato)borate, lithium hexafluorophosphate, lithium tetrafluoroborate, lithium perchlorate, lithium hexafluoroarsenate, lithium triflate, lithium nitrate, lithium difluorophosphate, lithium difluoro(oxalato)borate, lithium trifluoroacetate, lithium 4,5-dicyano-2-(trifluoromethyl)imidazole, lithium difluoro(dioxalato)phosphate, lithium tetrafluoro(oxalato)phosphate, lithium bis(pentafluoroethanesulfonyl)imide, or a combination thereof.

The present disclosure provides an electrolyte including 1,2-difluoroethyl 3,3,3-trifluoropropanoate, 1,1-difluoroethyl 3,3,3-trifluoropropanoate, 1,1-difluoroethyl 2,2-difluoroacetate, or a combination thereof; and sodium bis(trifluoromethanesulfonyl)imide, sodium bis(fluorosulfonyl)imide, sodium bis(oxalato)borate, sodium hexafluorophosphate, sodium tetrafluoroborate, sodium perchlorate, sodium hexafluoroarsenate, sodium triflate, sodium nitrate, sodium difluorophosphate, sodium difluoro(oxalato)borate, sodium trifluoroacetate, sodium 4,5-dicyano-2-(trifluoromethyl)imidazole, or a combination thereof.

The present disclosure provides an electrolyte including 1,2-difluoroethyl 3,3,3-trifluoropropanoate, 1,1-difluoroethyl 3,3,3-trifluoropropanoate, 1,1-difluoroethyl 2,2-difluoroacetate, or a combination thereof; fluoroethylene carbonate, propylene carbonate, dimethyl carbonate, diethyl carbonate, ethyl methyl carbonate, vinyl carbonate, vinyl ethylene carbonate, ethylene carbonate, difluoroethylene carbonate, 3,3,3-trifluoropropylene carbonate, monofluoroethyl methyl carbonate, difluoroethyl methyl carbonate, trifluoroethyl methyl carbonate, bis(2,2,2-trifluoroethyl) carbonate, 2,2-difluoroethyl ethyl carbonate, acetonitrile, fluoroacetonitrile, ethyl acetate, methyl acetate, methyl propanoate, methyl 3,3,3-trifluoropropanoate, ethyl 3,3,3-trifluoropropanoate, ethyl 2,2-difluoropropanoate, isopropyl trifluoroacetate, methyl 4,4,4-trifluorobutanoate, methyl difluoroacetate, 2,2,2-trifluoroethyl butanoate, methyl 2,2-difluoropropanoate, ethyl trifluoroacetate, 2,2-difluoroethyl trifluoroacetate, 2,2,2-trifluoroethyl acetate, ethyl 2,2-difluoroacetate, 2,2-difluoroethyl acetate, 2,2-difluoroethyl 3,3-difluoropropanoate, 2,2-difluoroethyl 2,2-difluoroacetate, succinonitrile, propionitrile, butyronitrile, adiponitrile, 1,3,6-hexanetricarbonitrile, trimethyl borate, triphenyl borate, triethyl borate, tris(pentafluorophenyl)borane, tris(trimethylsilyl)phosphate, tris(2,2,2-trifluoroethyl) borate, trimethyl phosphate, triethyl phosphate, tris(trimethylsilyl)phosphate, tris(trimethylsilyl)phosphite, tris(2,2,2-trifluoroethyl) phosphate, tris(2,2,2-trifluoroethyl) phosphite, (pentafluorophenyl)diphenyl phosphine, tris(pentafluorophenyl) phosphine, 1,3,2-dioxathiolane-2,2-dioxide, 1,3-propanesultone, prop-1-ene-1,3-sultone, propanediol cyclic sulfate, 1,3,2-dioxathiolane 2-oxide, bis(2,2,2-trifluoroethyl) ether, 1,1,2,2-tetrafluoroethyl-2,2,3,3-tetrafluoropropyl ether, 1H,1H,5H-octafluoropentyl-1,1,2,2-tetrafluoroethylether, 2,2,2-trifluoroethyl 1,1,2,2-tetrafluoroethyl ether, tetrahydrofuran, 1,3-dioxolane, 1,2-dimethoxyethane, diethylene glycol dimethyl ether, tetraethylene glycol dimethyl ether, succinic anhydride, butyric anhydride, 1,4-dioxane, tetravinyl silane, 1,4-butane sultone, dimethyl sulfoxide, methylene methanedisulfonate, N,N-dimethylformamide, gamma-butyrolactone, or a combination thereof; and a metal salt.

### BRIEF DESCRIPTION OF THE DRAWING

The above mentioned and other features of the disclosure, and the manner of attaining them, will become more apparent and the disclosure itself will be better understood by reference to the following description of embodiments of the disclosure taken in conjunction with the accompanying drawing.

The FIGURE is a semi-schematic drawing of one example of a battery having an electrolyte of the present disclosure.

The exemplification set out herein illustrates an embodiment of the disclosure, and such exemplification is not to be construed as limiting the scope of the disclosure in any manner.

### DETAILED DESCRIPTION

### I. Definitions

TABLE 1
Names of compounds used herein and corresponding
structures and chemical names

| Name | Structure and Chemical Name |
|---|---|
| Fluorinated Ester 1 | 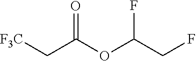 [SMILES: O=C(CC(F)(F)F)OC(F)CF] |
| | 1,2-difluoroethyl 3,3,3-trifluoropropanoate |
| Fluorinated Ester 2 | 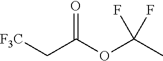 |
| | 1,1-difluoroethyl 3,3,3-trifluoropropanoate |
| Fluorinated Ester 3 | 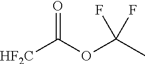 |
| | 1,1-difluoroethyl 2,2-difluoroacetate |

It is understood that the invention comprises, consists essentially of, or consists of the compounds identified in Table 1, alone or in combination.

As used herein, the singular forms “a”, “an”, and “the” include plural references unless the context clearly dictates otherwise. Thus, reference to “an” element in a claim followed by reference to “the” element is inclusive of one element and a plurality of the elements.

As used herein, the recitation of numerical ranges by endpoints includes all numbers subsumed within that range (e.g., 1 to 5 includes 1, 1.5, 2, 2.75, 3, 3.8, 4, and 5).

Unless otherwise indicated, all numbers expressing quantities or ingredients, measurement of properties and so forth used in the specification and embodiments are to be understood as being modified in all instances by the term “about.” Accordingly, unless indicated to the contrary, the numerical parameters set forth in the specification and attached listing of embodiments may vary depending upon the desired properties sought to be obtained by those skilled in the art utilizing the teachings of the present disclosure. At the very least, and not as an attempt to limit the application of the doctrine of equivalents to the scope of the claimed embodiments, each numerical parameter should at least be construed in light of the number of reported significant digits and by applying ordinary rounding techniques.

As used herein, the phrase “within any range encompassing any two of these values as endpoints” literally means that any range may be selected from any two of the values listed prior to such phrase regardless of whether the values are in the lower part of the listing or in the higher part of the listing. For example, a pair of values may be selected from two lower values, two higher values, or a lower value and a higher value.

### II. Synthesis of Fluorinated Esters 1-3

The present disclosure includes methods of synthesizing fluorinated esters. A general method of synthesizing a fluorinated ester includes providing a carboxylic acid or carboxylic acid derivative; and reacting the carboxylic acid or carboxylic acid derivative with a fluorinated olefin to synthesize the fluorinated ester. A general method of synthesizing a fluorinated ester includes providing a fluorinated carboxylic acid or fluorinated carboxylic acid derivative; and reacting the fluorinated carboxylic acid or fluorinated carboxylic acid derivative with a fluorinated olefin to synthesize the fluorinated ester.

Fluorinated carboxylic acids and carboxylic acid derivatives are used. Suitable carboxylic acids include 3,3,3-trifluoropropanoic acid, difluoroacetic acid. Suitable fluorinated carboxylic acids include 3,3,3-trifluoropropanoic acid and difluoroacetic acid. Suitable fluorinated carboxylic acid derivatives include derivatives of 3,3,3-trifluoropropanoic acid and difluoroacetic acid. Fluorinated carboxylic acid derivatives include fluorinated carboxylate salts and fluorinated hypochlorous anhydrides. Fluorinated esters for synthesis by the methods disclosed herein include 1,2-difluoroethyl 3,3,3-trifluoropropanoate (Fluorinated Ester 1), 1,1-difluoroethyl 3,3,3-trifluoropropanoate (Fluorinated Ester 2), and 1,1-difluoroethyl 2,2-difluoroacetate (Fluorinated Ester 3).

A synthetic route for synthesizing fluorinated esters includes providing fluorinated carboxylic acids or fluorinated carboxylic acid derivatives and reacting the fluorinated carboxylic acids or fluorinated carboxylic acid derivatives with fluorinated olefins to form fluorinated esters. Selective reductive dehalogenation of intermediate fluorinated esters is included for synthesis of the desired fluorinated esters. Specifically, reductive dechlorination of fluorinated ester intermediates is promoted by zinc. Optionally, a fluorinated sodium carboxylate salt can be reacted with chlorine monofluoride to form a fluorinated hypochlorous anhydride carboxylic acid derivative, which is reacted with a fluorinated olefin to synthesize a fluorinated ester intermediate, which undergoes selective reductive dehalogenation to provide the desired fluorinated ester. Synthetic examples 1A-2C disclosed herein describe synthesis of Fluorinated Esters 1-3 by the disclosed synthetic routes.

### III. Properties of Fluorinated Esters 1-3

Properties of Fluorinated Esters 1-3 including, dielectric constant, oxidation potential, dielectric constant, boiling point, and viscosity were calculated or simulated using computational methods. The properties suggest that Fluorinated Esters 1-3 will be beneficial as components of electrolytes for batteries, with different properties being more important than other properties, depending on a battery being formed and uses thereof. The properties of Fluorinated Esters 1-3 are provided in Table 2.

TABLE 2
Properties of Fluorinated Esters 1-3

| | Oxidation | | | Viscosity at |
|---|---|---|---|---|
| | Potential | Dielectric | Boiling | 25° C. |
| | (volts (V) | Constant | Point | (centipoise |
| Fluorinated Ester | vs. Li/Li⁺) | (Dk) | (° C.) | (cP)) |
| 1,2-difluoroethyl 3,3,3- | 7.5 | 7.7 | 155 | 2.8 |
| trifluoropropanoate | | | | |
| (Fluorinated Ester 1) | | | | |
| 1,1-difluoroethyl 3,3,3- | 7.4 | 18.1 | 116 | 1.4 |
| trifluoropropanoate | | | | |
| (Fluorinated Ester 2) | | | | |
| 1,1-difluoroethyl 2,2- | 7.7 | 26.2 | 90 | 0.88 |
| difluoroacetate | | | | |
| (Fluorinated Ester 3) | | | | |

Oxidation potential is a measure of tendency to lose electrons and become oxidized at an electrode. Electrolytes and electrolyte components with high oxidation potentials can withstand high battery voltages without degrading via oxidation reactions. Oxidation potentials were computed using quantum chemistry for molecular dimers in implicit acetone solvent at the M05-2X/6-31+g(d,p) level of theory (Delp, Samuel A., Oleg Borodin, Marco Olguin, Claire G. Eisner, Joshua L. Allen, and T. Richard Jow, “Importance of reduction and oxidation stability of high voltage electrolytes and additives,” Electrochimica Acta 209 (2016): 498-510). Fluorinated Esters 1-3 have calculated oxidation potentials greater than 7 V vs. Li/Li⁺, suggesting usefulness in batteries with high operating voltages. Oxidation potentials can be measured using standard electrochemical techniques, including cyclic voltammetry or potentiostatic holds.

Dielectric constant (Dk) is a measure of ability to store electrical energy in an electric field. A higher dielectric constant of a solvent correlates with a higher ability of the solvent to dissolve salts, e.g., a higher solubility, for example, to form an electrolyte. Dielectric constants were computed using molecular dynamics simulations with the OpenFF 2.1.0 Sage force field (Wang, Lily, Pavan Kumar Behara, Matthew W. Thompson, Trevor Gokey, Yuanqing Wang, Jeffrey R. Wagner, Daniel J. Cole, Michael K. Gilson, Michael R. Shirts, and David L. Mobley, “The open force field initiative: Open software and open science for molecular modeling,” The Journal of Physical Chemistry B 128, no. 29 (2024): 7043-7067). Fluorinated Esters 1-3 have computed dielectric constants greater than 5, suggesting that Fluorinated Esters 1-3 will enable the dissolution of lithium or sodium salts to form an electrolyte. Dielectric constant can be measured using the open-ended coaxial probe technique, while the solubility of salts in a solvent can be measured using solvent addition or temperature variation methods.

Solvents for use in electrolytes have boiling points higher than the operating temperature of the battery to prevent gas generation within the battery. Boiling points were computed using COSMO-RS method as implemented in BIOVIA COSMOTherm (https://www.3ds.com/products/biovia/cosmo-rs/cosmotherm). Computed boiling points of Fluorinated Esters 1-3 are greater than or equal to 90° C., suggesting suitability as electrolyte components in battery applications operated at ambient temperature to greater than or equal to 80° C. Computed boiling points of Fluorinated Esters 1-3 are greater than or equal to 75° C., suggesting suitability as electrolyte components in battery applications operated at ambient temperature to greater than or equal to 65° C. Boiling points can be measured using an ebulliometer device, while gassing from batteries can be quantified using techniques based on Archimedes' principle.

Viscosity is a measure of resistance of a fluid to movement in neighboring portions relative to one another. Electrolyte viscosity impacts ion transport properties, with lower viscosity correlated to higher ionic conductivity and greater battery (dis)charge rate capability. As further disclosed herein, low viscosity allows ions within the electrolyte to move more freely, enhancing conductivity of the electrolyte, and improving operation of the electrolyte, and a battery including the same, at low temperatures. Viscosities were computed using QSPR models as implemented in BIOVIA COSMOTherm (https://www.3ds.com/products/biovia/cosmo-rs/cosmotherm). The computed viscosities of Fluorinated Esters 1-3 are less than or equal to 3 cP at 25° C., suggesting that electrolytes including Fluorinated Esters 1-3 will have suitable viscosity for lithium-ion and sodium-ion batteries. Viscosity can be measured using various types of viscometers, including capillary and rotational viscometers.

The number of fluorine atoms in a fluorinated ester affects flammability, with more fluorine atoms corresponding with desirable reduced flammability. The flammability of electrolytes can be characterized using standard techniques including open and closed cup flash point measurements.

### IV. Electrolytes

The present disclosure provides electrolytes including at least one of Fluorinated Esters 1-3 and a metal salt. The metal salt can be a lithium salt, a sodium salt, or a combination thereof.

### A. Metal Salt

### 1. Lithium Salt

The lithium salt can include lithium bis(trifluoromethanesulfonyl)imide (LiTFSI) (also referred to herein as Lithium Salt 1), lithium bis(fluorosulfonyl)imide (LiFSI) (also referred to herein as Lithium Salt 2), lithium bis(oxalato)borate (LiBOB) (also referred to herein as Lithium Salt 3), lithium hexafluorophosphate (LiPF₆) (also referred to herein as Lithium Salt 4), lithium tetrafluoroborate (LiBF₄) (also referred to herein as Lithium Salt 5), lithium perchlorate (LiClO₄) (also referred to herein as Lithium Salt 6), lithium hexafluoroarsenate (LiAsF₆) (also referred to herein as Lithium Salt 7), lithium triflate (LiTf) (also referred to herein as Lithium Salt 8), lithium nitrate (LiNO₃) (also referred to herein as Lithium Salt 9), lithium difluorophosphate (LiDFP) (also referred to herein as Lithium Salt 10), lithium difluoro(oxalato)borate (LiDFOB) (also referred to herein as Lithium Salt 11), lithium trifluoroacetate (LiTFA) (also referred to herein as Lithium Salt 12), lithium 4,5-dicyano-2-(trifluoromethyl)imidazole (LiTDI) (also referred to herein as Lithium Salt 13), lithium difluoro(dioxalato)phosphate (LiDFDOP) (also referred to herein as Lithium Salt 14), lithium tetrafluoro(oxalato)phosphate (LiTFOP) (also referred to herein as Lithium Salt 15), lithium bis(pentafluoroethanesulfonyl)imide (LiBETI) (also referred to herein as Lithium Salt 16), or a combination thereof. The concentration of the lithium salt can be from about 0.01 moles per liter (molar, M) to about 5 M, from about 0.01 M to about 0.1 M, from about 0.3 M to about 2.5 M, from about 0.7 M to about 1.5 M, or within any range encompassed by any two of the foregoing values as endpoints. The concentration of lithium salt can be selected depending on the battery being formed and uses thereof.

Tables 3-5 define electrolytes including Fluorinated Ester 1, Fluorinated Ester 2, or Fluorinated Ester 3, respectively, and at least one lithium salt. The first column of each table identifies and defines the fluorinated ester (FE) by number as Fluorinated Ester 1, Fluorinated Ester 2, etc., and the Lithium Salt (LS) by number as Lithium Salt 1, Lithium Salt 2, etc. In the second column, the lithium salt is identified. The third column indicates the concentration of the lithium salt present in the electrolyte.

TABLE 3

| Fluorinated Ester | Lithium Salt | Lithium Salt Concentration |
|---|---|---|
| Fluorinated Ester 1 | LiTFSI | 0.01M-5M |
| Fluorinated Ester 1 | LiTFSI | 0.01M-0.1M |
| Fluorinated Ester 1 | LiTFSI | 0.3M-2.5M |
| Fluorinated Ester 1 | LiTFSI | 0.7M-1.5M |
| Fluorinated Ester 1 | LiFSI | 0.01M-5M |
| Fluorinated Ester 1 | LiFSI | 0.01M-0.1M |
| Fluorinated Ester 1 | LiFSI | 0.3M-2.5M |
| Fluorinated Ester 1 | LiFSI | 0.7M-1.5M |
| Fluorinated Ester 1 | LiBOB | 0.01M-5M |
| Fluorinated Ester 1 | LiBOB | 0.01M-0.1M |
| Fluorinated Ester 1 | LiBOB | 0.3M-2.5M |
| Fluorinated Ester 1 | LiBOB | 0.7M-1.5M |
| Fluorinated Ester 1 | LiPF₆ | 0.01M-5M |
| Fluorinated Ester 1 | LiPF₆ | 0.01M-0.1M |
| Fluorinated Ester 1 | LiPF₆ | 0.3M-2.5M |
| Fluorinated Ester 1 | LiPF₆ | 0.7M-1.5M |
| Fluorinated Ester 1 | LiBF₄ | 0.01M-5M |
| Fluorinated Ester 1 | LiBF₄ | 0.01M-0.1M |
| Fluorinated Ester 1 | LiBF₄ | 0.3M-2.5M |
| Fluorinated Ester 1 | LiBF₄ | 0.7M-1.5M |
| Fluorinated Ester 1 | LiClO₄ | 0.01M-5M |
| Fluorinated Ester 1 | LiClO₄ | 0.01M-0.1M |
| Fluorinated Ester 1 | LiClO₄ | 0.3M-2.5M |
| Fluorinated Ester 1 | LiClO₄ | 0.7M-1.5M |
| Fluorinated Ester 1 | LiAsF₆ | 0.01M-5M |
| Fluorinated Ester 1 | LiAsF₆ | 0.01M-0.1M |
| Fluorinated Ester 1 | LiAsF₆ | 0.3M-2.5M |
| Fluorinated Ester 1 | LiAsF₆ | 0.7M-1.5M |
| Fluorinated Ester 1 | LiTf | 0.01M-5M |
| Fluorinated Ester 1 | LiTf | 0.01M-0.1M |
| Fluorinated Ester 1 | LiTf | 0.3M-2.5M |
| Fluorinated Ester 1 | LiTf | 0.7M-1.5M |
| Fluorinated Ester 1 | LiNO₃ | 0.01M-5M |
| Fluorinated Ester 1 | LiNO₃ | 0.01M-0.1M |
| Fluorinated Ester 1 | LiNO₃ | 0.3M-2.5M |
| Fluorinated Ester 1 | LiNO₃ | 0.7M-1.5M |
| Fluorinated Ester 1 | LiDFP | 0.01M-5M |
| Fluorinated Ester 1 | LiDFP | 0.01M-0.1M |
| Fluorinated Ester 1 | LiDFP | 0.3M-2.5M |
| Fluorinated Ester 1 | LiDFP | 0.7M-1.5M |
| Fluorinated Ester 1 | LiDFOB | 0.01M-5M |
| Fluorinated Ester 1 | LiDFOB | 0.01M-0.1M |
| Fluorinated Ester 1 | LiDFOB | 0.3M-2.5M |
| Fluorinated Ester 1 | LiDFOB | 0.7M-1.5M |
| Fluorinated Ester 1 | LiTFA | 0.01M-5M |
| Fluorinated Ester 1 | LiTFA | 0.01M-0.1M |
| Fluorinated Ester 1 | LiTFA | 0.3M-2.5M |
| Fluorinated Ester 1 | LiTFA | 0.7M-1.5M |
| Fluorinated Ester 1 | LiTDI | 0.01M-5M |
| Fluorinated Ester 1 | LiTDI | 0.01M-0.1M |
| Fluorinated Ester 1 | LiTDI | 0.3M-2.5M |
| Fluorinated Ester 1 | LiTDI | 0.7M-1.5M |
| Fluorinated Ester 1 | LiDFDOP | 0.01M-5M |
| Fluorinated Ester 1 | LiDFDOP | 0.01M-0.1M |
| Fluorinated Ester 1 | LiDFDOP | 0.3M-2.5M |
| Fluorinated Ester 1 | LiDFDOP | 0.7M-1.5M |
| Fluorinated Ester 1 | LiTFOP | 0.01M-5M |
| Fluorinated Ester 1 | LiTFOP | 0.01M-0.1M |
| Fluorinated Ester 1 | LiTFOP | 0.3M-2.5M |
| Fluorinated Ester 1 | LiTFOP | 0.7M-1.5M |
| Fluorinated Ester 1 | LiBETI | 0.01M-5M |
| Fluorinated Ester 1 | LiBETI | 0.01M-0.1M |
| Fluorinated Ester 1 | LiBETI | 0.3M-2.5M |
| Fluorinated Ester 1 | LiBETI | 0.7M-1.5M |

TABLE 4

| Fluorinated Ester | Lithium Salt | Lithium Salt Concentration |
|---|---|---|
| Fluorinated Ester 2 | LiTFSI | 0.01M-5M |
| Fluorinated Ester 2 | LiTFSI | 0.01M-0.1M |
| Fluorinated Ester 2 | LiTFSI | 0.3M-2.5M |
| Fluorinated Ester 2 | LiTFSI | 0.7M-1.5M |
| Fluorinated Ester 2 | LiFSI | 0.01M-5M |
| Fluorinated Ester 2 | LiFSI | 0.01M-0.1M |
| Fluorinated Ester 2 | LiFSI | 0.3M-2.5M |
| Fluorinated Ester 2 | LiFSI | 0.7M-1.5M |
| Fluorinated Ester 2 | LiBOB | 0.01M-5M |
| Fluorinated Ester 2 | LiBOB | 0.01M-0.1M |
| Fluorinated Ester 2 | LiBOB | 0.3M-2.5M |
| Fluorinated Ester 2 | LiBOB | 0.7M-1.5M |
| Fluorinated Ester 2 | LiPF₆ | 0.01M-5M |
| Fluorinated Ester 2 | LiPF₆ | 0.01M-0.1M |
| Fluorinated Ester 2 | LiPF₆ | 0.3M-2.5M |
| Fluorinated Ester 2 | LiPF₆ | 0.7M-1.5M |
| Fluorinated Ester 2 | LiBF₄ | 0.01M-5M |
| Fluorinated Ester 2 | LiBF₄ | 0.01M-0.1M |
| Fluorinated Ester 2 | LiBF₄ | 0.3M-2.5M |
| Fluorinated Ester 2 | LiBF₄ | 0.7M-1.5M |
| Fluorinated Ester 2 | LiClO₄ | 0.01M-5M |
| Fluorinated Ester 2 | LiClO₄ | 0.01M-0.1M |
| Fluorinated Ester 2 | LiClO₄ | 0.3M-2.5M |
| Fluorinated Ester 2 | LiClO₄ | 0.7M-1.5M |
| Fluorinated Ester 2 | LiAsF₆ | 0.01M-5M |
| Fluorinated Ester 2 | LiAsF₆ | 0.01M-0.1M |
| Fluorinated Ester 2 | LiAsF₆ | 0.3M-2.5M |
| Fluorinated Ester 2 | LiAsF₆ | 0.7M-1.5M |
| Fluorinated Ester 2 | LiTf | 0.01M-5M |
| Fluorinated Ester 2 | LiTf | 0.01M-0.1M |
| Fluorinated Ester 2 | LiTf | 0.3M-2.5M |
| Fluorinated Ester 2 | LiTf | 0.7M-1.5M |
| Fluorinated Ester 2 | LiNO₃ | 0.01M-5M |
| Fluorinated Ester 2 | LiNO₃ | 0.01M-0.1M |
| Fluorinated Ester 2 | LiNO₃ | 0.3M-2.5M |
| Fluorinated Ester 2 | LiNO₃ | 0.7M-1.5M |
| Fluorinated Ester 2 | LiDFP | 0.01M-5M |
| Fluorinated Ester 2 | LiDFP | 0.01M-0.1M |
| Fluorinated Ester 2 | LiDFP | 0.3M-2.5M |
| Fluorinated Ester 2 | LiDFP | 0.7M-1.5M |
| Fluorinated Ester 2 | LiDFOB | 0.01M-5M |
| Fluorinated Ester 2 | LiDFOB | 0.01M-0.1M |
| Fluorinated Ester 2 | LiDFOB | 0.3M-2.5M |
| Fluorinated Ester 2 | LiDFOB | 0.7M-1.5M |
| Fluorinated Ester 2 | LiTFA | 0.01M-5M |
| Fluorinated Ester 2 | LiTFA | 0.01M-0.1M |
| Fluorinated Ester 2 | LiTFA | 0.3M-2.5M |
| Fluorinated Ester 2 | LiTFA | 0.7M-1.5M |
| Fluorinated Ester 2 | LiTDI | 0.01M-5M |
| Fluorinated Ester 2 | LiTDI | 0.01M-0.1M |
| Fluorinated Ester 2 | LiTDI | 0.3M-2.5M |
| Fluorinated Ester 2 | LiTDI | 0.7M-1.5M |
| Fluorinated Ester 2 | LiDFDOP | 0.01M-5M |
| Fluorinated Ester 2 | LiDFDOP | 0.01M-0.1M |
| Fluorinated Ester 2 | LiDFDOP | 0.3M-2.5M |
| Fluorinated Ester 2 | LiDFDOP | 0.7M-1.5M |
| Fluorinated Ester 2 | LiTFOP | 0.01M-5M |
| Fluorinated Ester 2 | LiTFOP | 0.01M-0.1M |
| Fluorinated Ester 2 | LiTFOP | 0.3M-2.5M |
| Fluorinated Ester 2 | LiTFOP | 0.7M-1.5M |
| Fluorinated Ester 2 | LiBETI | 0.01M-5M |
| Fluorinated Ester 2 | LiBETI | 0.01M-0.1M |
| Fluorinated Ester 2 | LiBETI | 0.3M-2.5M |
| Fluorinated Ester 2 | LiBETI | 0.7M-1.5M |

TABLE 5

| Fluorinated Ester | Lithium Salt | Lithium Salt Concentration |
|---|---|---|
| Fluorinated Ester 3 | LiTFSI | 0.01M-5M |
| Fluorinated Ester 3 | LiTFSI | 0.01M-0.1M |
| Fluorinated Ester 3 | LiTFSI | 0.3M-2.5M |
| Fluorinated Ester 3 | LiTFSI | 0.7M-1.5M |
| Fluorinated Ester 3 | LiFSI | 0.01M-5M |
| Fluorinated Ester 3 | LiFSI | 0.01M-0.1M |
| Fluorinated Ester 3 | LiFSI | 0.3M-2.5M |
| Fluorinated Ester 3 | LiFSI | 0.7M-1.5M |
| Fluorinated Ester 3 | LiBOB | 0.01M-5M |
| Fluorinated Ester 3 | LiBOB | 0.01M-0.1M |
| Fluorinated Ester 3 | LiBOB | 0.3M-2.5M |
| Fluorinated Ester 3 | LiBOB | 0.7M-1.5M |
| Fluorinated Ester 3 | LiPF₆ | 0.01M-5M |
| Fluorinated Ester 3 | LiPF₆ | 0.01M-0.1M |
| Fluorinated Ester 3 | LiPF₆ | 0.3M-2.5M |
| Fluorinated Ester 3 | LiPF₆ | 0.7M-1.5M |
| Fluorinated Ester 3 | LiBF₄ | 0.01M-5M |
| Fluorinated Ester 3 | LiBF₄ | 0.01M-0.1M |
| Fluorinated Ester 3 | LiBF₄ | 0.3M-2.5M |
| Fluorinated Ester 3 | LiBF₄ | 0.7M-1.5M |
| Fluorinated Ester 3 | LiClO₄ | 0.01M-5M |
| Fluorinated Ester 3 | LiClO₄ | 0.01M-0.1M |
| Fluorinated Ester 3 | LiClO₄ | 0.3M-2.5M |
| Fluorinated Ester 3 | LiClO₄ | 0.7M-1.5M |
| Fluorinated Ester 3 | LiAsF₆ | 0.01M-5M |
| Fluorinated Ester 3 | LiAsF₆ | 0.01M-0.1M |
| Fluorinated Ester 3 | LiAsF₆ | 0.3M-2.5M |
| Fluorinated Ester 3 | LiAsF₆ | 0.7M-1.5M |
| Fluorinated Ester 3 | LiTf | 0.01M-5M |
| Fluorinated Ester 3 | LiTf | 0.01M-0.1M |
| Fluorinated Ester 3 | LiTf | 0.3M-2.5M |
| Fluorinated Ester 3 | LiTf | 0.7M-1.5M |
| Fluorinated Ester 3 | LiNO₃ | 0.01M-5M |
| Fluorinated Ester 3 | LiNO₃ | 0.01M-0.1M |
| Fluorinated Ester 3 | LiNO₃ | 0.3M-2.5M |
| Fluorinated Ester 3 | LiNO₃ | 0.7M-1.5M |
| Fluorinated Ester 3 | LiDFP | 0.01M-5M |
| Fluorinated Ester 3 | LiDFP | 0.01M-0.1M |
| Fluorinated Ester 3 | LiDFP | 0.3M-2.5M |
| Fluorinated Ester 3 | LiDFP | 0.7M-1.5M |
| Fluorinated Ester 3 | LiDFOB | 0.01M-5M |
| Fluorinated Ester 3 | LiDFOB | 0.01M-0.1M |
| Fluorinated Ester 3 | LiDFOB | 0.3M-2.5M |
| Fluorinated Ester 3 | LiDFOB | 0.7M-1.5M |
| Fluorinated Ester 3 | LiTFA | 0.01M-5M |
| Fluorinated Ester 3 | LiTFA | 0.01M-0.1M |
| Fluorinated Ester 3 | LiTFA | 0.3M-2.5M |
| Fluorinated Ester 3 | LiTFA | 0.7M-1.5M |
| Fluorinated Ester 3 | LiTDI | 0.01M-5M |
| Fluorinated Ester 3 | LiTDI | 0.01M-0.1M |
| Fluorinated Ester 3 | LiTDI | 0.3M-2.5M |
| Fluorinated Ester 3 | LiTDI | 0.7M-1.5M |
| Fluorinated Ester 3 | LiDFDOP | 0.01M-5M |
| Fluorinated Ester 3 | LiDFDOP | 0.01M-0.1M |
| Fluorinated Ester 3 | LiDFDOP | 0.3M-2.5M |
| Fluorinated Ester 3 | LiDFDOP | 0.7M-1.5M |
| Fluorinated Ester 3 | LiTFOP | 0.01M-5M |
| Fluorinated Ester 3 | LiTFOP | 0.01M-0.1M |
| Fluorinated Ester 3 | LiTFOP | 0.3M-2.5M |
| Fluorinated Ester 3 | LiTFOP | 0.7M-1.5M |
| Fluorinated Ester 3 | LiBETI | 0.01M-5M |
| Fluorinated Ester 3 | LiBETI | 0.01M-0.1M |
| Fluorinated Ester 3 | LiBETI | 0.3M-2.5M |
| Fluorinated Ester 3 | LiBETI | 0.7M-1.5M |

It is understood that the invention comprises, consists essentially of, or consists of the compositions identified in Table 3. It is understood that the invention comprises, consists essentially of, or consists of the compositions identified in Table 4. It is understood that the invention comprises, consists essentially of, or consists of the compositions identified in Table 5.

The electrolyte can include more than one lithium salt, for example, two lithium salts, three lithium salts, or four or more lithium salts. Exemplary combinations of more than one lithium salt include, for example, LiFSI and LiPF₆; or LiDFOB and one or more of LiPF₆, LiTFSI, LiFSI, or LiBF₄. With reference to Tables 3-5, when a listed electrolyte further includes one or more additional lithium salt(s) in addition to the listed lithium salt, the listed lithium salt concentration can refer to the total lithium salt concentration or the lithium salt concentration of each included lithium salt. A combination of lithium salts, concentrations thereof, or a combination thereof can be selected depending on a battery being formed and uses thereof.

### 2. Sodium Salts

The sodium salt can include sodium bis(trifluoromethanesulfonyl)imide (NaTFSI) (also referred to herein as Sodium Salt 1), sodium bis(fluorosulfonyl)imide (NaFSI) (also referred to herein as Sodium Salt 2), sodium bis(oxalato)borate (NaBOB) (also referred to herein as Sodium Salt 3), sodium hexafluorophosphate (NaPF₆) (also referred to herein as Sodium Salt 4), sodium tetrafluoroborate (NaBF₄) (also referred to herein as Sodium Salt 5), sodium perchlorate (NaClO₄) (also referred to herein as Sodium Salt 6), sodium hexafluoroarsenate (NaAsF₆) (also referred to herein as Sodium Salt 7), sodium triflate (NaTf) (also referred to herein as Sodium Salt 8), sodium nitrate (NaNO₃) (also referred to herein as Sodium Salt 9), sodium difluorophosphate (NaDFP) (also referred to herein as Sodium Salt 10), sodium difluoro(oxalato)borate (NaDFOB) (also referred to herein as Sodium Salt 11), sodium trifluoroacetate (NaTFA) (also referred to herein as Sodium Salt 12), sodium 4,5-dicyano-2-(trifluoromethyl)imidazole (NaTDI) (also referred to herein as Sodium Salt 13), or a combination thereof. The concentration of the sodium salt can be from about 0.01 M to about 5 M, from about 0.01 M to about 0.1 M, from about 0.3 M to about 2.5 M, from about 0.7 M to about 1.5 M, or within any range encompassed by any two of the foregoing values as endpoints.

Tables 6-8 define electrolytes including Fluorinated Ester 1, Fluorinated Ester 2, or Fluorinated Ester 3, respectively, and at least one sodium salt. The first column of each table identifies and defines the fluorinated ester (FE) by number as Fluorinated Ester 1, Fluorinated Ester 2, etc., and the Sodium Salt (SS) by number as Sodium Salt 1, Sodium Salt 2, etc. In the second column, the sodium salt is identified. The third column indicates the concentration of the sodium salt present in the electrolyte.

TABLE 6

| Fluorinated Ester | Sodium Salt | Sodium Salt Concentration |
|---|---|---|
| Fluorinated Ester 1 | NaTFSI | 0.01M-5M |
| Fluorinated Ester 1 | NaTFSI | 0.01M-0.1M |
| Fluorinated Ester 1 | NaTFSI | 0.3M-2.5M |
| Fluorinated Ester 1 | NaTFSI | 0.7M-1.5M |
| Fluorinated Ester 1 | NaFSI | 0.01M-5M |
| Fluorinated Ester 1 | NaFSI | 0.01M-0.1M |
| Fluorinated Ester 1 | NaFSI | 0.3M-2.5M |
| Fluorinated Ester 1 | NaFSI | 0.7M-1.5M |
| Fluorinated Ester 1 | NaBOB | 0.01M-5M |
| Fluorinated Ester 1 | NaBOB | 0.01M-0.1M |
| Fluorinated Ester 1 | NaBOB | 0.3M-2.5M |
| Fluorinated Ester 1 | NaBOB | 0.7M-1.5M |
| Fluorinated Ester 1 | NaPF₆ | 0.01M-5M |
| Fluorinated Ester 1 | NaPF₆ | 0.01M-0.1M |
| Fluorinated Ester 1 | NaPF₆ | 0.3M-2.5M |
| Fluorinated Ester 1 | NaPF₆ | 0.7M-1.5M |
| Fluorinated Ester 1 | NaBF₄ | 0.01M-5M |
| Fluorinated Ester 1 | NaBF₄ | 0.01M-0.1M |
| Fluorinated Ester 1 | NaBF₄ | 0.3M-2.5M |
| Fluorinated Ester 1 | NaBF₄ | 0.7M-1.5M |
| Fluorinated Ester 1 | NaClO₄ | 0.01M-5M |
| Fluorinated Ester 1 | NaClO₄ | 0.01M-0.1M |
| Fluorinated Ester 1 | NaClO₄ | 0.3M-2.5M |
| Fluorinated Ester 1 | NaClO₄ | 0.7M-1.5M |
| Fluorinated Ester 1 | NaAsF₆ | 0.01M-5M |
| Fluorinated Ester 1 | NaAsF₆ | 0.01M-0.1M |
| Fluorinated Ester 1 | NaAsF₆ | 0.3M-2.5M |
| Fluorinated Ester 1 | NaAsF₆ | 0.7M-1.5M |
| Fluorinated Ester 1 | NaTf | 0.01M-5M |
| Fluorinated Ester 1 | NaTf | 0.01M-0.1M |
| Fluorinated Ester 1 | NaTf | 0.3M-2.5M |
| Fluorinated Ester 1 | NaTf | 0.7M-1.5M |
| Fluorinated Ester 1 | NaNO₃ | 0.01M-5M |
| Fluorinated Ester 1 | NaNO₃ | 0.01M-0.1M |
| Fluorinated Ester 1 | NaNO₃ | 0.3M-2.5M |
| Fluorinated Ester 1 | NaNO₃ | 0.7M-1.5M |
| Fluorinated Ester 1 | NaDFP | 0.01M-5M |
| Fluorinated Ester 1 | NaDFP | 0.01M-0.1M |
| Fluorinated Ester 1 | NaDFP | 0.3M-2.5M |
| Fluorinated Ester 1 | NaDFP | 0.7M-1.5M |
| Fluorinated Ester 1 | NaDFOB | 0.01M-5M |
| Fluorinated Ester 1 | NaDFOB | 0.01M-0.1M |
| Fluorinated Ester 1 | NaDFOB | 0.3M-2.5M |
| Fluorinated Ester 1 | NaDFOB | 0.7M-1.5M |
| Fluorinated Ester 1 | NaTFA | 0.01M-5M |
| Fluorinated Ester 1 | NaTFA | 0.01M-0.1M |
| Fluorinated Ester 1 | NaTFA | 0.3M-2.5M |
| Fluorinated Ester 1 | NaTFA | 0.7M-1.5M |
| Fluorinated Ester 1 | NaTDI | 0.01M-5M |
| Fluorinated Ester 1 | NaTDI | 0.01M-0.1M |
| Fluorinated Ester 1 | NaTDI | 0.3M-2.5M |
| Fluorinated Ester 1 | NaTDI | 0.7M-1.5M |

TABLE 7

| Fluorinated Ester | Sodium Salt | Sodium Salt Concentration |
|---|---|---|
| Fluorinated Ester 2 | NaTFSI | 0.01M-5M |
| Fluorinated Ester 2 | NaTFSI | 0.01M-0.1M |
| Fluorinated Ester 2 | NaTFSI | 0.3M-2.5M |
| Fluorinated Ester 2 | NaTFSI | 0.7M-1.5M |
| Fluorinated Ester 2 | NaFSI | 0.01M-5M |
| Fluorinated Ester 2 | NaFSI | 0.01M-0.1M |
| Fluorinated Ester 2 | NaFSI | 0.3M-2.5M |
| Fluorinated Ester 2 | NaFSI | 0.7M-1.5M |
| Fluorinated Ester 2 | NaBOB | 0.01M-5M |
| Fluorinated Ester 2 | NaBOB | 0.01M-0.1M |
| Fluorinated Ester 2 | NaBOB | 0.3M-2.5M |
| Fluorinated Ester 2 | NaBOB | 0.7M-1.5M |
| Fluorinated Ester 2 | NaPF₆ | 0.01M-5M |
| Fluorinated Ester 2 | NaPF₆ | 0.01M-0.1M |
| Fluorinated Ester 2 | NaPF₆ | 0.3M-2.5M |
| Fluorinated Ester 2 | NaPF₆ | 0.7M-1.5M |
| Fluorinated Ester 2 | NaBF₄ | 0.01M-5M |
| Fluorinated Ester 2 | NaBF₄ | 0.01M-0.1M |
| Fluorinated Ester 2 | NaBF₄ | 0.3M-2.5M |
| Fluorinated Ester 2 | NaBF₄ | 0.7M-1.5M |
| Fluorinated Ester 2 | NaClO₄ | 0.01M-5M |
| Fluorinated Ester 2 | NaClO₄ | 0.01M-0.1M |
| Fluorinated Ester 2 | NaClO₄ | 0.3M-2.5M |
| Fluorinated Ester 2 | NaClO₄ | 0.7M-1.5M |
| Fluorinated Ester 2 | NaAsF₆ | 0.01M-5M |
| Fluorinated Ester 2 | NaAsF₆ | 0.01M-0.1M |
| Fluorinated Ester 2 | NaAsF₆ | 0.3M-2.5M |
| Fluorinated Ester 2 | NaAsF₆ | 0.7M-1.5M |
| Fluorinated Ester 2 | NaTf | 0.01M-5M |
| Fluorinated Ester 2 | NaTf | 0.01M-0.1M |
| Fluorinated Ester 2 | NaTf | 0.3M-2.5M |
| Fluorinated Ester 2 | NaTf | 0.7M-1.5M |
| Fluorinated Ester 2 | NaNO₃ | 0.01M-5M |
| Fluorinated Ester 2 | NaNO₃ | 0.01M-0.1M |
| Fluorinated Ester 2 | NaNO₃ | 0.3M-2.5M |
| Fluorinated Ester 2 | NaNO₃ | 0.7M-1.5M |
| Fluorinated Ester 2 | NaDFP | 0.01M-5M |
| Fluorinated Ester 2 | NaDFP | 0.01M-0.1M |
| Fluorinated Ester 2 | NaDFP | 0.3M-2.5M |
| Fluorinated Ester 2 | NaDFP | 0.7M-1.5M |
| Fluorinated Ester 2 | NaDFOB | 0.01M-5M |
| Fluorinated Ester 2 | NaDFOB | 0.01M-0.1M |
| Fluorinated Ester 2 | NaDFOB | 0.3M-2.5M |
| Fluorinated Ester 2 | NaDFOB | 0.7M-1.5M |
| Fluorinated Ester 2 | NaTFA | 0.01M-5M |
| Fluorinated Ester 2 | NaTFA | 0.01M-0.1M |
| Fluorinated Ester 2 | NaTFA | 0.3M-2.5M |
| Fluorinated Ester 2 | NaTFA | 0.7M-1.5M |
| Fluorinated Ester 2 | NaTDI | 0.01M-5M |
| Fluorinated Ester 2 | NaTDI | 0.01M-0.1M |
| Fluorinated Ester 2 | NaTDI | 0.3M-2.5M |
| Fluorinated Ester 2 | NaTDI | 0.7M-1.5M |

TABLE 8

| Fluorinated Ester | Sodium Salt | Sodium Salt Concentration |
|---|---|---|
| Fluorinated Ester 3 | NaTFSI | 0.01M-5M |
| Fluorinated Ester 3 | NaTFSI | 0.01M-0.1M |
| Fluorinated Ester 3 | NaTFSI | 0.3M-2.5M |
| Fluorinated Ester 3 | NaTFSI | 0.7M-1.5M |
| Fluorinated Ester 3 | NaFSI | 0.01M-5M |
| Fluorinated Ester 3 | NaFSI | 0.01M-0.1M |
| Fluorinated Ester 3 | NaFSI | 0.3M-2.5M |
| Fluorinated Ester 3 | NaFSI | 0.7M-1.5M |
| Fluorinated Ester 3 | NaBOB | 0.01M-5M |
| Fluorinated Ester 3 | NaBOB | 0.01M-0.1M |
| Fluorinated Ester 3 | NaBOB | 0.3M-2.5M |
| Fluorinated Ester 3 | NaBOB | 0.7M-1.5M |
| Fluorinated Ester 3 | NaPF₆ | 0.01M-5M |
| Fluorinated Ester 3 | NaPF₆ | 0.01M-0.1M |
| Fluorinated Ester 3 | NaPF₆ | 0.3M-2.5M |
| Fluorinated Ester 3 | NaPF₆ | 0.7M-1.5M |
| Fluorinated Ester 3 | NaBF₄ | 0.01M-5M |
| Fluorinated Ester 3 | NaBF₄ | 0.01M-0.1M |
| Fluorinated Ester 3 | NaBF₄ | 0.3M-2.5M |
| Fluorinated Ester 3 | NaBF₄ | 0.7M-1.5M |
| Fluorinated Ester 3 | NaClO₄ | 0.01M-5M |
| Fluorinated Ester 3 | NaClO₄ | 0.01M-0.1M |
| Fluorinated Ester 3 | NaClO₄ | 0.3M-2.5M |
| Fluorinated Ester 3 | NaClO₄ | 0.7M-1.5M |
| Fluorinated Ester 3 | NaAsF₆ | 0.01M-5M |
| Fluorinated Ester 3 | NaAsF₆ | 0.01M-0.1M |
| Fluorinated Ester 3 | NaAsF₆ | 0.3M-2.5M |
| Fluorinated Ester 3 | NaAsF₆ | 0.7M-1.5M |
| Fluorinated Ester 3 | NaTf | 0.01M-5M |
| Fluorinated Ester 3 | NaTf | 0.01M-0.1M |
| Fluorinated Ester 3 | NaTf | 0.3M-2.5M |
| Fluorinated Ester 3 | NaTf | 0.7M-1.5M |
| Fluorinated Ester 3 | NaNO₃ | 0.01M-5M |
| Fluorinated Ester 3 | NaNO₃ | 0.01M-0.1M |
| Fluorinated Ester 3 | NaNO₃ | 0.3M-2.5M |
| Fluorinated Ester 3 | NaNO₃ | 0.7M-1.5M |
| Fluorinated Ester 3 | NaDFP | 0.01M-5M |
| Fluorinated Ester 3 | NaDFP | 0.01M-0.1M |
| Fluorinated Ester 3 | NaDFP | 0.3M-2.5M |
| Fluorinated Ester 3 | NaDFP | 0.7M-1.5M |
| Fluorinated Ester 3 | NaDFOB | 0.01M-5M |
| Fluorinated Ester 3 | NaDFOB | 0.01M-0.1M |
| Fluorinated Ester 3 | NaDFOB | 0.3M-2.5M |
| Fluorinated Ester 3 | NaDFOB | 0.7M-1.5M |
| Fluorinated Ester 3 | NaTFA | 0.01M-5M |
| Fluorinated Ester 3 | NaTFA | 0.01M-0.1M |
| Fluorinated Ester 3 | NaTFA | 0.3M-2.5M |
| Fluorinated Ester 3 | NaTFA | 0.7M-1.5M |
| Fluorinated Ester 3 | NaTDI | 0.01M-5M |
| Fluorinated Ester 3 | NaTDI | 0.01M-0.1M |
| Fluorinated Ester 3 | NaTDI | 0.3M-2.5M |
| Fluorinated Ester 3 | NaTDI | 0.7M-1.5M |

It is understood that the invention comprises, consists essentially of, or consists of the compositions identified in Table 6. It is understood that the invention comprises, consists essentially of, or consists of the compositions identified in Table 7. It is understood that the invention comprises, consists essentially of, or consists of the compositions identified in Table 8.

The electrolyte can include more than one sodium salt, for example, two sodium salts, three sodium salts, or four or more sodium salts. Exemplary combinations of more than one sodium salt include, for example, NaFSI and NaPF₆; or NaDFOB and one or more of NaPF₆, NaTFSI, NaFSI, or NaBF₄. With reference to Tables 6-8, when a listed electrolyte further includes one or more additional sodium salt(s) in addition to the listed sodium salt, the listed sodium salt concentration can refer to the total sodium salt concentration or the sodium salt concentration of each included sodium salt. A combination of sodium salts, concentrations thereof, or a combination thereof can be selected depending on a battery being formed and uses thereof.

### B. Supplemental Solvent

Electrolytes including at least one of Fluorinated Esters 1-3 can include a supplemental solvent, e.g., at least one, solvent that is not one of Fluorinated Esters 1-3. Exemplary reasons for including Fluorinated Esters 1-3, at least one supplemental solvent, or a combination thereof in an electrolyte include to adjust electrolyte viscosity, to improve ability to solvate, to form an interphase at a cathode or anode of a battery including the electrolyte, to function as a hydrofluoric acid scavenger, and to function as a safety additive.

For example, an electrolyte including at least one of Fluorinated Esters 1-3, at least one supplemental solvent, or a combination thereof can allow for a lower viscosity of the electrolyte, for example, at low temperatures, than would be exhibited by an electrolyte lacking at least one of Fluorinated Esters 1-3, at least one supplemental solvent, or a combination thereof. A lower viscosity allows ions within the electrolyte to move more freely, enhancing conductivity of the electrolyte.

Fluorinated Esters 1-3, at least one supplemental solvent, or a combination thereof can serve as a film-forming additive that can create a protective film on a surface of an electrode of a battery during operation of the battery. The film can improve performance, e.g., cycling performance, of the battery by stabilizing an interface between the electrolyte and the electrode. During the first charging cycle, the film-forming additive may decompose and deposit an interphase layer, called a solid electrolyte interphase (SEI) on the anode, and called a cathode electrolyte interphase (CEI) on the cathode. The SEI, CEI, or a combination thereof can prevent the electrolyte from directly contacting the charged electrodes.

Tables 9A-9C includes such solvents that are not one of Fluorinated Esters 1-3 that can be included in the electrolyte. The amount of supplemental solvent in the electrolyte can be from about 0.5 wt. % to about 99.0 wt. %, based on a total weight of the electrolyte. Fluorinated Esters 1-3 can be present in an amount of from 0.1 wt. % to 5 wt. % (e.g., as an additive), from greater than 5 wt. % to 25 wt. % (e.g., as a secondary solvent), or from greater than 25 wt. % to 90 wt. % (e.g., as a primary solvent), or within any range encompassed by any two of the foregoing values as endpoints, based on a total weight of the electrolyte. The amount of Fluorinated Esters 1-3 can be selected depending on a battery being formed and uses thereof. It is understood that the invention comprises, consists essentially of, or consists of the compositions identified in Table 9A. It is understood that the invention comprises, consists essentially of, or consists of the compositions identified in Table 9B. It is understood that the invention comprises, consists essentially of, or consists of the compositions identified in Table 9C.

TABLE 9A

| | | Concentration of |
|---|---|---|
| Fluorinated Ester | Solvent | Fluorinated Ester 1 |
| Fluorinated Ester 1 | fluoroethylene carbonate (FEC) | 0.1 wt. %-5 wt. % |
| Fluorinated Ester 1 | fluoroethylene carbonate (FEC) | >5 wt. %-25 wt. % |
| Fluorinated Ester 1 | fluoroethylene carbonate (FEC) | >25 wt. %-95 wt. % |
| Fluorinated Ester 1 | propylene carbonate (PC) | 0.1 wt. %-5 wt. % |
| Fluorinated Ester 1 | propylene carbonate (PC) | >5 wt. %-25 wt. % |
| Fluorinated Ester 1 | propylene carbonate (PC) | >25 wt. %-95 wt. % |
| Fluorinated Ester 1 | dimethyl carbonate (DMC) | 0.1 wt. %-5 wt. % |
| Fluorinated Ester 1 | dimethyl carbonate (DMC) | >5 wt. %-25 wt. % |
| Fluorinated Ester 1 | dimethyl carbonate (DMC) | >25 wt. %-95 wt. % |
| Fluorinated Ester 1 | diethyl carbonate (DEC) | 0.1 wt. %-5 wt. % |
| Fluorinated Ester 1 | diethyl carbonate (DEC) | >5 wt. %-25 wt. % |
| Fluorinated Ester 1 | diethyl carbonate (DEC) | >25 wt. %-95 wt. % |
| Fluorinated Ester 1 | ethyl Methyl carbonate (EMC) | 0.1 wt. %-5 wt. % |
| Fluorinated Ester 1 | ethyl Methyl carbonate (EMC) | >5 wt. %-25 wt. % |
| Fluorinated Ester 1 | ethyl Methyl carbonate (EMC) | >25 wt. %-95 wt. % |
| Fluorinated Ester 1 | vinyl carbonate (VC) | 0.1 wt. %-5 wt. % |
| Fluorinated Ester 1 | vinyl carbonate (VC) | >5 wt. %-25 wt. % |
| Fluorinated Ester 1 | vinyl carbonate (VC) | >25 wt. %-95 wt. % |
| Fluorinated Ester 1 | vinyl ethylene carbonate (VEC) | 0.1 wt. %-5 wt. % |
| Fluorinated Ester 1 | vinyl ethylene carbonate (VEC) | >5 wt. %-25 wt. % |
| Fluorinated Ester 1 | vinyl ethylene carbonate (VEC) | >25 wt. %-95 wt. % |
| Fluorinated Ester 1 | ethylene carbonate (EC) | 0.1 wt. %-5 wt. % |
| Fluorinated Ester 1 | ethylene carbonate (EC) | >5 wt. %-25 wt. % |
| Fluorinated Ester 1 | ethylene carbonate (EC) | >25 wt. %-95 wt. % |
| Fluorinated Ester 1 | difluoroethylene carbonate (DFEC) | 0.1 wt. %-5 wt. % |
| Fluorinated Ester 1 | difluoroethylene carbonate (DFEC) | >5 wt. %-25 wt. % |
| Fluorinated Ester 1 | difluoroethylene carbonate (DFEC) | >25 wt. %-95 wt. % |
| Fluorinated Ester 1 | 3,3,3-trifluoropropylene carbonate | 0.1 wt. %-5 wt. % |
| | (TFPC) | |
| Fluorinated Ester 1 | 3,3,3-trifluoropropylene carbonate | >5 wt. %-25 wt. % |
| | (TFPC) | |
| Fluorinated Ester 1 | 3,3,3-trifluoropropylene carbonate | >25 wt. %-95 wt. % |
| | (TFPC) | |
| Fluorinated Ester 1 | monofluoroethyl Methyl carbonate | 0.1 wt. %-5 wt. % |
| | (F1EMC) | |
| Fluorinated Ester 1 | monofluoroethyl Methyl carbonate | >5 wt. %-25 wt. % |
| | (F1EMC) | |
| Fluorinated Ester 1 | monofluoroethyl Methyl carbonate | >25 wt. %-95 wt. % |
| | (F1EMC) | |
| Fluorinated Ester 1 | difluoroethyl Methyl carbonate (F2EMC) | 0.1 wt. %-5 wt. % |
| Fluorinated Ester 1 | difluoroethyl Methyl carbonate (F2EMC) | >5 wt. %-25 wt. % |
| Fluorinated Ester 1 | difluoroethyl Methyl carbonate (F2EMC) | >25 wt. %-95 wt. % |
| Fluorinated Ester 1 | trifluoroethyl Methyl carbonate (F3EMC) | 0.1 wt. %-5 wt. % |
| Fluorinated Ester 1 | trifluoroethyl Methyl carbonate (F3EMC) | >5 wt. %-25 wt. % |
| Fluorinated Ester 1 | trifluoroethyl Methyl carbonate (F3EMC) | >25 wt. %-95 wt. % |
| Fluorinated Ester 1 | bis(2,2,2-trifluoroethyl) carbonate (TFEC) | 0.1 wt. %-5 wt. % |
| Fluorinated Ester 1 | bis(2,2,2-trifluoroethyl) carbonate (TFEC) | >5 wt. %-25 wt. % |
| Fluorinated Ester 1 | bis(2,2,2-trifluoroethyl) carbonate (TFEC) | >25 wt. %-95 wt. % |
| Fluorinated Ester 1 | 2,2-difluoroethyl ethyl carbonate | 0.1 wt. %-5 wt. % |
| | (DFDEC) | |
| Fluorinated Ester 1 | 2,2-difluoroethyl ethyl carbonate | >5 wt. %-25 wt. % |
| | (DFDEC) | |
| Fluorinated Ester 1 | 2,2-difluoroethyl ethyl carbonate | >25 wt. %-95 wt. % |
| | (DFDEC) | |
| Fluorinated Ester 1 | acetonitrile (AN) | 0.1 wt. %-5 wt. % |
| Fluorinated Ester 1 | acetonitrile (AN) | >5 wt. %-25 wt. % |
| Fluorinated Ester 1 | acetonitrile (AN) | >25 wt. %-95 wt. % |
| Fluorinated Ester 1 | fluoroacetonitrile | 0.1 wt. %-5 wt. % |
| Fluorinated Ester 1 | fluoroacetonitrile | >5 wt. %-25 wt. % |
| Fluorinated Ester 1 | fluoroacetonitrile | >25 wt. %-95 wt. % |
| Fluorinated Ester 1 | ethyl acetate (EA) | 0.1 wt. %-5 wt. % |
| Fluorinated Ester 1 | ethyl acetate (EA) | >5 wt. %-25 wt. % |
| Fluorinated Ester 1 | ethyl acetate (EA) | >25 wt. %-95 wt. % |
| Fluorinated Ester 1 | methyl acetate (MA) | 0.1 wt. %-5 wt. % |
| Fluorinated Ester 1 | methyl acetate (MA) | >5 wt. %-25 wt. % |
| Fluorinated Ester 1 | methyl acetate (MA) | >25 wt. %-95 wt. % |
| Fluorinated Ester 1 | methyl propanoate (MP) | 0.1 wt. %-5 wt. % |
| Fluorinated Ester 1 | methyl propanoate (MP) | >5 wt. %-25 wt. % |
| Fluorinated Ester 1 | methyl propanoate (MP) | >25 wt. %-95 wt. % |
| Fluorinated Ester 1 | methyl 3,3,3-trifluoropropanoate | 0.1 wt. %-5 wt. % |
| Fluorinated Ester 1 | methyl 3,3,3-trifluoropropanoate | >5 wt. %-25 wt. % |
| Fluorinated Ester 1 | methyl 3,3,3-trifluoropropanoate | >25 wt. %-95 wt. % |
| Fluorinated Ester 1 | ethyl 3,3,3-trifluoropropanoate | 0.1 wt. %-5 wt. % |
| Fluorinated Ester 1 | ethyl 3,3,3-trifluoropropanoate | >5 wt. %-25 wt. % |
| Fluorinated Ester 1 | ethyl 3,3,3-trifluoropropanoate | >25 wt. %-95 wt. % |
| Fluorinated Ester 1 | ethyl 2,2-difluoropropanoate | 0.1 wt. %-5 wt. % |
| Fluorinated Ester 1 | ethyl 2,2-difluoropropanoate | >5 wt. %-25 wt. % |
| Fluorinated Ester 1 | ethyl 2,2-difluoropropanoate | >25 wt. %-95 wt. % |
| Fluorinated Ester 1 | isopropyl trifluoroacetate | 0.1 wt. %-5 wt. % |
| Fluorinated Ester 1 | isopropyl trifluoroacetate | >5 wt. %-25 wt. % |
| Fluorinated Ester 1 | isopropyl trifluoroacetate | >25 wt. %-95 wt. % |
| Fluorinated Ester 1 | methyl 4,4,4-trifluorobutanoate | 0.1 wt. %-5 wt. % |
| Fluorinated Ester 1 | methyl 4,4,4-trifluorobutanoate | >5 wt. %-25 wt. % |
| Fluorinated Ester 1 | methyl 4,4,4-trifluorobutanoate | >25 wt. %-95 wt. % |
| Fluorinated Ester 1 | methyl difluoroacetate | 0.1 wt. %-5 wt. % |
| Fluorinated Ester 1 | methyl difluoroacetate | >5 wt. %-25 wt. % |
| Fluorinated Ester 1 | methyl difluoroacetate | >25 wt. %-95 wt. % |
| Fluorinated Ester 1 | 2,2,2-trifluoroethyl butanoate | 0.1 wt. %-5 wt. % |
| Fluorinated Ester 1 | 2,2,2-trifluoroethyl butanoate | >5 wt. %-25 wt. % |
| Fluorinated Ester 1 | 2,2,2-trifluoroethyl butanoate | >25 wt. %-95 wt. % |
| Fluorinated Ester 1 | methyl 2,2-difluoropropanoate | 0.1 wt. %-5 wt. % |
| Fluorinated Ester 1 | methyl 2,2-difluoropropanoate | >5 wt. %-25 wt. % |
| Fluorinated Ester 1 | methyl 2,2-difluoropropanoate | >25 wt. %-95 wt. % |
| Fluorinated Ester 1 | ethyl trifluoroacetate | 0.1 wt. %-5 wt. % |
| Fluorinated Ester 1 | ethyl trifluoroacetate | >5 wt. %-25 wt. % |
| Fluorinated Ester 1 | ethyl trifluoroacetate | >25 wt. %-95 wt. % |
| Fluorinated Ester 1 | 2,2-difluoroethyl trifluoroacetate | 0.1 wt. %-5 wt. % |
| Fluorinated Ester 1 | 2,2-difluoroethyl trifluoroacetate | >5 wt. %-25 wt. % |
| Fluorinated Ester 1 | 2,2-difluoroethyl trifluoroacetate | >25 wt. %-95 wt. % |
| Fluorinated Ester 1 | 2,2,2-trifluoroethyl acetate | 0.1 wt. %-5 wt. % |
| Fluorinated Ester 1 | 2,2,2-trifluoroethyl acetate | >5 wt. %-25 wt. % |
| Fluorinated Ester 1 | 2,2,2-trifluoroethyl acetate | >25 wt. %-95 wt. % |
| Fluorinated Ester 1 | ethyl 2,2-difluoroacetate | 0.1 wt. %-5 wt. % |
| Fluorinated Ester 1 | ethyl 2,2-difluoroacetate | >5 wt. %-25 wt. % |
| Fluorinated Ester 1 | ethyl 2,2-difluoroacetate | >25 wt. %-95 wt. % |
| Fluorinated Ester 1 | 2,2-difluoroethyl acetate | 0.1 wt. %-5 wt. % |
| Fluorinated Ester 1 | 2,2-difluoroethyl acetate | >5 wt. %-25 wt. % |
| Fluorinated Ester 1 | 2,2-difluoroethyl acetate | >25 wt. %-95 wt. % |
| Fluorinated Ester 1 | 2,2-difluoroethyl 3,3-difluoropropanoate | 0.1 wt. %-5 wt. % |
| Fluorinated Ester 1 | 2,2-difluoroethyl 3,3-difluoropropanoate | >5 wt. %-25 wt. % |
| Fluorinated Ester 1 | 2,2-difluoroethyl 3,3-difluoropropanoate | >25 wt. %-95 wt. % |
| Fluorinated Ester 1 | 2,2-difluoroethyl 2,2-difluoroacetate | 0.1 wt. %-5 wt. % |
| Fluorinated Ester 1 | 2,2-difluoroethyl 2,2-difluoroacetate | >5 wt. %-25 wt. % |
| Fluorinated Ester 1 | 2,2-difluoroethyl 2,2-difluoroacetate | >25 wt. %-95 wt. % |
| Fluorinated Ester 1 | succinonitrile (SN) | 0.1 wt. %-5 wt. % |
| Fluorinated Ester 1 | succinonitrile (SN) | >5 wt. %-25 wt. % |
| Fluorinated Ester 1 | succinonitrile (SN) | >25 wt. %-95 wt. % |
| Fluorinated Ester 1 | propionitrile (PN) | 0.1 wt. %-5 wt. % |
| Fluorinated Ester 1 | propionitrile (PN) | >5 wt. %-25 wt. % |
| Fluorinated Ester 1 | propionitrile (PN) | >25 wt. %-95 wt. % |
| Fluorinated Ester 1 | butyronitrile (BN) | 0.1 wt. %-5 wt. % |
| Fluorinated Ester 1 | butyronitrile (BN) | >5 wt. %-25 wt. % |
| Fluorinated Ester 1 | butyronitrile (BN) | >25 wt. %-95 wt. % |
| Fluorinated Ester 1 | adiponitrile (ADN) | 0.1 wt. %-5 wt. % |
| Fluorinated Ester 1 | adiponitrile (ADN) | >5 wt. %-25 wt. % |
| Fluorinated Ester 1 | adiponitrile (ADN) | >25 wt. %-95 wt. % |
| Fluorinated Ester 1 | 1,3,6-hexanetricarbonitrile (HTCN) | 0.1 wt. %-5 wt. % |
| Fluorinated Ester 1 | 1,3,6-hexanetricarbonitrile (HTCN) | >5 wt. %-25 wt. % |
| Fluorinated Ester 1 | 1,3,6-hexanetricarbonitrile (HTCN) | >25 wt. %-95 wt. % |
| Fluorinated Ester 1 | trimethyl borate (TMB) | 0.1 wt. %-5 wt. % |
| Fluorinated Ester 1 | trimethyl borate (TMB) | >5 wt. %-25 wt. % |
| Fluorinated Ester 1 | trimethyl borate (TMB) | >25 wt. %-95 wt. % |
| Fluorinated Ester 1 | triphenyl borate (TPB) | 0.1 wt. %-5 wt. % |
| Fluorinated Ester 1 | triphenyl borate (TPB) | >5 wt. %-25 wt. % |
| Fluorinated Ester 1 | triphenyl borate (TPB) | >25 wt. %-95 wt. % |
| Fluorinated Ester 1 | triethyl borate (TEB) | 0.1 wt. %-5 wt. % |
| Fluorinated Ester 1 | triethyl borate (TEB) | >5 wt. %-25 wt. % |
| Fluorinated Ester 1 | triethyl borate (TEB) | >25 wt. %-95 wt. % |
| Fluorinated Ester 1 | tris(pentafluorophenyl)borane (TPFPB) | 0.1 wt. %-5 wt. % |
| Fluorinated Ester 1 | tris(pentafluorophenyl)borane (TPFPB) | >5 wt. %-25 wt. % |
| Fluorinated Ester 1 | tris(pentafluorophenyl)borane (TPFPB) | >25 wt. %-95 wt. % |
| Fluorinated Ester 1 | tris(trimethylsilyl)phosphate (TTSB) | 0.1 wt. %-5 wt. % |
| Fluorinated Ester 1 | tris(trimethylsilyl)phosphate (TTSB) | >5 wt. %-25 wt. % |
| Fluorinated Ester 1 | tris(trimethylsilyl)phosphate (TTSB) | >25 wt. %-95 wt. % |
| Fluorinated Ester 1 | tris(2,2,2-trifluoroethyl) borate (TTFEB) | 0.1 wt. %-5 wt. % |
| Fluorinated Ester 1 | tris(2,2,2-trifluoroethyl) borate (TTFEB) | >5 wt. %-25 wt. % |
| Fluorinated Ester 1 | tris(2,2,2-trifluoroethyl) borate (TTFEB) | >25 wt. %-95 wt. % |
| Fluorinated Ester 1 | trimethyl phosphate (TMP) | 0.1 wt. %-5 wt. % |
| Fluorinated Ester 1 | trimethyl phosphate (TMP) | >5 wt. %-25 wt. % |
| Fluorinated Ester 1 | trimethyl phosphate (TMP) | >25 wt. %-95 wt. % |
| Fluorinated Ester 1 | triethyl phosphate (TEP) | 0.1 wt. %-5 wt. % |
| Fluorinated Ester 1 | triethyl phosphate (TEP) | >5 wt. %-25 wt. % |
| Fluorinated Ester 1 | triethyl phosphate (TEP) | >25 wt. %-95 wt. % |
| Fluorinated Ester 1 | tris(trimethylsilyl)phosphate (TTSP) | 0.1 wt. %-5 wt. % |
| Fluorinated Ester 1 | tris(trimethylsilyl)phosphate (TTSP) | >5 wt. %-25 wt. % |
| Fluorinated Ester 1 | tris(trimethylsilyl)phosphate (TTSP) | >25 wt. %-95 wt. % |
| Fluorinated Ester 1 | tris(trimethylsilyl)phosphite (TTSPi) | 0.1 wt. %-5 wt. % |
| Fluorinated Ester 1 | tris(trimethylsilyl)phosphite (TTSPi) | >5 wt. %-25 wt. % |
| Fluorinated Ester 1 | tris(trimethylsilyl)phosphite (TTSPi) | >25 wt. %-95 wt. % |
| Fluorinated Ester 1 | tris(2,2,2-trifluoroethyl) phosphate | 0.1 wt. %-5 wt. % |
| | (TFEPa) | |
| Fluorinated Ester 1 | tris(2,2,2-trifluoroethyl) phosphate | >5 wt. %-25 wt. % |
| | (TFEPa) | |
| Fluorinated Ester 1 | tris(2,2,2-trifluoroethyl) phosphate | >25 wt. %-95 wt. % |
| | (TFEPa) | |
| Fluorinated Ester 1 | tris(2,2,2-trifluoroethyl) phosphite | 0.1 wt. %-5 wt. % |
| | (TFEPi) | |
| Fluorinated Ester 1 | tris(2,2,2-trifluoroethyl) phosphite | >5 wt. %-25 wt. % |
| | (TFEPi) | |
| Fluorinated Ester 1 | tris(2,2,2-trifluoroethyl) phosphite | >25 wt. %-95 wt. % |
| | (TFEPi) | |
| Fluorinated Ester 1 | (pentafluorophenyl)diphenyl phosphine | 0.1 wt. %-5 wt. % |
| | (PFPDPP) | |
| Fluorinated Ester 1 | (pentafluorophenyl)diphenyl phosphine | >5 wt. %-25 wt. % |
| | (PFPDPP) | |
| Fluorinated Ester 1 | (pentafluorophenyl)diphenyl phosphine | >25 wt. %-95 wt. % |
| | (PFPDPP) | |
| Fluorinated Ester 1 | tris(pentafluorophenyl) phosphine | 0.1 wt. %-5 wt. % |
| | (TPFPP) | |
| Fluorinated Ester 1 | tris(pentafluorophenyl) phosphine | >5 wt. %-25 wt. % |
| | (TPFPP) | |
| Fluorinated Ester 1 | tris(pentafluorophenyl) phosphine | >25 wt. %-95 wt. % |
| | (TPFPP) | |
| Fluorinated Ester 1 | 1,3,2-dioxathiolane-2,2-dioxide (DTD) | 0.1 wt. %-5 wt. % |
| Fluorinated Ester 1 | 1,3,2-dioxathiolane-2,2-dioxide (DTD) | >5 wt. %-25 wt. % |
| Fluorinated Ester 1 | 1,3,2-dioxathiolane-2,2-dioxide (DTD) | >25 wt. %-95 wt. % |
| Fluorinated Ester 1 | 1,3-propanesultone (PS) | 0.1 wt. %-5 wt. % |
| Fluorinated Ester 1 | 1,3-propanesultone (PS) | >5 wt. %-25 wt. % |
| Fluorinated Ester 1 | 1,3-propanesultone (PS) | >25 wt. %-95 wt. % |
| Fluorinated Ester 1 | prop-1-ene-1,3-sultone (PES) | 0.1 wt. %-5 wt. % |
| Fluorinated Ester 1 | prop-1-ene-1,3-sultone (PES) | >5 wt. %-25 wt. % |
| Fluorinated Ester 1 | prop-1-ene-1,3-sultone (PES) | >25 wt. %-95 wt. % |
| Fluorinated Ester 1 | propanediol cyclic sulfate (PCS) | 0.1 wt. %-5 wt. % |
| Fluorinated Ester 1 | propanediol cyclic sulfate (PCS) | >5 wt. %-25 wt. % |
| Fluorinated Ester 1 | propanediol cyclic sulfate (PCS) | >25 wt. %-95 wt. % |
| Fluorinated Ester 1 | 1,3,2-dioxathiolane 2-oxide | 0.1 wt. %-5 wt. % |
| Fluorinated Ester 1 | 1,3,2-dioxathiolane 2-oxide | >5 wt. %-25 wt. % |
| Fluorinated Ester 1 | 1,3,2-dioxathiolane 2-oxide | >25 wt. %-95 wt. % |
| Fluorinated Ester 1 | bis(2,2,2-trifluoroethyl) ether (BTFE) | 0.1 wt. %-5 wt. % |
| Fluorinated Ester 1 | bis(2,2,2-trifluoroethyl) ether (BTFE) | >5 wt. %-25 wt. % |
| Fluorinated Ester 1 | bis(2,2,2-trifluoroethyl) ether (BTFE) | >25 wt. %-95 wt. % |
| Fluorinated Ester 1 | 1,1,2,2-tetrafluoroethyl-2,2,3,3- | 0.1 wt. %-5 wt. % |
| | tetrafluoropropyl ether (TTE) | |
| Fluorinated Ester 1 | 1,1,2,2-tetrafluoroethyl-2,2,3,3- | >5 wt. %-25 wt. % |
| | tetrafluoropropyl ether (TTE) | |
| Fluorinated Ester 1 | 1,1,2,2-tetrafluoroethyl-2,2,3,3- | >25 wt. %-95 wt. % |
| | tetrafluoropropyl ether (TTE) | |
| Fluorinated Ester 1 | 1H,1H,5H-octafluoropentyl-1,1,2,2- | 0.1 wt. %-5 wt. % |
| | tetrafluoroethylether (OTE) | |
| Fluorinated Ester 1 | 1H,1H,5H-octafluoropentyl-1,1,2,2- | >5 wt. %-25 wt. % |
| | tetrafluoroethylether (OTE) | |
| Fluorinated Ester 1 | 1H,1H,5H-octafluoropentyl-1,1,2,2- | >25 wt. %-95 wt. % |
| | tetrafluoroethylether (OTE) | |
| Fluorinated Ester 1 | 2,2,2-trifluoroethyl 1,1,2,2- | 0.1 wt. %-5 wt. % |
| | tetrafluoroethyl ether (HFE) | |
| Fluorinated Ester 1 | 2,2,2-trifluoroethyl 1,1,2,2- | >5 wt. %-25 wt. % |
| | tetrafluoroethyl ether (HFE) | |
| Fluorinated Ester 1 | 2,2,2-trifluoroethyl 1,1,2,2- | >25 wt. %-95 wt. % |
| | tetrafluoroethyl ether (HFE) | |
| Fluorinated Ester 1 | tetrahydrofuran (THF) | 0.1 wt. %-5 wt. % |
| Fluorinated Ester 1 | tetrahydrofuran (THF) | >5 wt. %-25 wt. % |
| Fluorinated Ester 1 | tetrahydrofuran (THF) | >25 wt. %-95 wt. % |
| Fluorinated Ester 1 | 1,3-dioxolane (DOL) | 0.1 wt. %-5 wt. % |
| Fluorinated Ester 1 | 1,3-dioxolane (DOL) | >5 wt. %-25 wt. % |
| Fluorinated Ester 1 | 1,3-dioxolane (DOL) | >25 wt. %-95 wt. % |
| Fluorinated Ester 1 | 1,2-dimethoxyethane (DME) | 0.1 wt. %-5 wt. % |
| Fluorinated Ester 1 | 1,2-dimethoxyethane (DME) | >5 wt. %-25 wt. % |
| Fluorinated Ester 1 | 1,2-dimethoxyethane (DME) | >25 wt. %-95 wt. % |
| Fluorinated Ester 1 | diethylene glycol dimethyl ether | 0.1 wt. %-5 wt. % |
| | (DEGDME) | |
| Fluorinated Ester 1 | diethylene glycol dimethyl ether | >5 wt. %-25 wt. % |
| | (DEGDME) | |
| Fluorinated Ester 1 | diethylene glycol dimethyl ether | >25 wt. %-95 wt. % |
| | (DEGDME) | |
| Fluorinated Ester 1 | tetraethylene glycol dimethyl ether | 0.1 wt. %-5 wt. % |
| | (TEGDME) | |
| Fluorinated Ester 1 | tetraethylene glycol dimethyl ether | >5 wt. %-25 wt. % |
| | (TEGDME) | |
| Fluorinated Ester 1 | tetraethylene glycol dimethyl ether | >25 wt. %-95 wt. % |
| | (TEGDME) | |
| Fluorinated Ester 1 | succinic anhydride (SA) | 0.1 wt. %-5 wt. % |
| Fluorinated Ester 1 | succinic anhydride (SA) | >5 wt. %-25 wt. % |
| Fluorinated Ester 1 | succinic anhydride (SA) | >25 wt. %-95 wt. % |
| Fluorinated Ester 1 | butyric anhydride (BA) | 0.1 wt. %-5 wt. % |
| Fluorinated Ester 1 | butyric anhydride (BA) | >5 wt. %-25 wt. % |
| Fluorinated Ester 1 | butyric anhydride (BA) | >25 wt. %-95 wt. % |
| Fluorinated Ester 1 | 1,4-dioxane (DOX) | 0.1 wt. %-5 wt. % |
| Fluorinated Ester 1 | 1,4-dioxane (DOX) | >5 wt. %-25 wt. % |
| Fluorinated Ester 1 | 1,4-dioxane (DOX) | >25 wt. %-95 wt. % |
| Fluorinated Ester 1 | tetravinyl silane (TVSI) | 0.1 wt. %-5 wt. % |
| Fluorinated Ester 1 | tetravinyl silane (TVSI) | >5 wt. %-25 wt. % |
| Fluorinated Ester 1 | tetravinyl silane (TVSI) | >25 wt. %-95 wt. % |
| Fluorinated Ester 1 | 1,4-butane sultone (BS) | 0.1 wt. %-5 wt. % |
| Fluorinated Ester 1 | 1,4-butane sultone (BS) | >5 wt. %-25 wt. % |
| Fluorinated Ester 1 | 1,4-butane sultone (BS) | >25 wt. %-95 wt. % |
| Fluorinated Ester 1 | dimethyl sulfoxide (DMSO) | 0.1 wt. %-5 wt. % |
| Fluorinated Ester 1 | dimethyl sulfoxide (DMSO) | >5 wt. %-25 wt. % |
| Fluorinated Ester 1 | dimethyl sulfoxide (DMSO) | >25 wt. %-95 wt. % |
| Fluorinated Ester 1 | methylene Methanedisulfonate (MMDS) | 0.1 wt. %-5 wt. % |
| Fluorinated Ester 1 | methylene Methanedisulfonate (MMDS) | >5 wt. %-25 wt. % |
| Fluorinated Ester 1 | methylene Methanedisulfonate (MMDS) | >25 wt. %-95 wt. % |
| Fluorinated Ester 1 | N,N-dimethylformamide (DMF) | 0.1 wt. %-5 wt. % |
| Fluorinated Ester 1 | N,N-dimethylformamide (DMF) | >5 wt. %-25 wt. % |
| Fluorinated Ester 1 | N,N-dimethylformamide (DMF) | >25 wt. %-95 wt. % |
| Fluorinated Ester 1 | gamma-butyrolactone (BL) | 0.1 wt. %-5 wt. % |
| Fluorinated Ester 1 | gamma-butyrolactone (BL) | >5 wt. %-25 wt. % |
| Fluorinated Ester 1 | gamma-butyrolactone (BL) | >25 wt. %-95 wt. % |

TABLE 9B

| | | Concentration of |
|---|---|---|
| Fluorinated Ester | Solvent | Fluorinated Ester 2 |
| Fluorinated Ester 2 | fluoroethylene carbonate (FEC) | 0.1 wt. %-5 wt. % |
| Fluorinated Ester 2 | fluoroethylene carbonate (FEC) | >5 wt. %-25 wt. % |
| Fluorinated Ester 2 | fluoroethylene carbonate (FEC) | >25 wt. %-95 wt. % |
| Fluorinated Ester 2 | propylene carbonate (PC) | 0.1 wt. %-5 wt. % |
| Fluorinated Ester 2 | propylene carbonate (PC) | >5 wt. %-25 wt. % |
| Fluorinated Ester 2 | propylene carbonate (PC) | >25 wt. %-95 wt. % |
| Fluorinated Ester 2 | dimethyl carbonate (DMC) | 0.1 wt. %-5 wt. % |
| Fluorinated Ester 2 | dimethyl carbonate (DMC) | >5 wt. %-25 wt. % |
| Fluorinated Ester 2 | dimethyl carbonate (DMC) | >25 wt. %-95 wt. % |
| Fluorinated Ester 2 | diethyl carbonate (DEC) | 0.1 wt. %-5 wt. % |
| Fluorinated Ester 2 | diethyl carbonate (DEC) | >5 wt. %-25 wt. % |
| Fluorinated Ester 2 | diethyl carbonate (DEC) | >25 wt. %-95 wt. % |
| Fluorinated Ester 2 | ethyl methyl carbonate (EMC) | 0.1 wt. %-5 wt. % |
| Fluorinated Ester 2 | ethyl methyl carbonate (EMC) | >5 wt. %-25 wt. % |
| Fluorinated Ester 2 | ethyl methyl carbonate (EMC) | >25 wt. %-95 wt. % |
| Fluorinated Ester 2 | vinyl carbonate (VC) | 0.1 wt. %-5 wt. % |
| Fluorinated Ester 2 | vinyl carbonate (VC) | >5 wt. %-25 wt. % |
| Fluorinated Ester 2 | vinyl carbonate (VC) | >25 wt. %-95 wt. % |
| Fluorinated Ester 2 | vinyl ethylene carbonate (VEC) | 0.1 wt. %-5 wt. % |
| Fluorinated Ester 2 | vinyl ethylene carbonate (VEC) | >5 wt. %-25 wt. % |
| Fluorinated Ester 2 | vinyl ethylene carbonate (VEC) | >25 wt. %-95 wt. % |
| Fluorinated Ester 2 | ethylene carbonate (EC) | 0.1 wt. %-5 wt. % |
| Fluorinated Ester 2 | ethylene carbonate (EC) | >5 wt. %-25 wt. % |
| Fluorinated Ester 2 | ethylene carbonate (EC) | >25 wt. %-95 wt. % |
| Fluorinated Ester 2 | difluoroethylene carbonate (DFEC) | 0.1 wt. %-5 wt. % |
| Fluorinated Ester 2 | difluoroethylene carbonate (DFEC) | >5 wt. %-25 wt. % |
| Fluorinated Ester 2 | difluoroethylene carbonate (DFEC) | >25 wt. %-95 wt. % |
| Fluorinated Ester 2 | 3,3,3-trifluoropropylene carbonate | 0.1 wt. %-5 wt. % |
| | (TFPC) | |
| Fluorinated Ester 2 | 3,3,3-trifluoropropylene carbonate | >5 wt. %-25 wt. % |
| | (TFPC) | |
| Fluorinated Ester 2 | 3,3,3-trifluoropropylene carbonate | >25 wt. %-95 wt. % |
| | (TFPC) | |
| Fluorinated Ester 2 | monofluoroethyl methyl carbonate | 0.1 wt. %-5 wt. % |
| | (F1EMC) | |
| Fluorinated Ester 2 | monofluoroethyl methyl carbonate | >5 wt. %-25 wt. % |
| | (F1EMC) | |
| Fluorinated Ester 2 | monofluoroethyl methyl carbonate | >25 wt. %-95 wt. % |
| | (F1EMC) | |
| Fluorinated Ester 2 | difluoroethyl methyl carbonate | 0.1 wt. %-5 wt. % |
| | (F2EMC) | |
| Fluorinated Ester 2 | difluoroethyl methyl carbonate | >5 wt. %-25 wt. % |
| | (F2EMC) | |
| Fluorinated Ester 2 | difluoroethyl methyl carbonate | >25 wt. %-95 wt. % |
| | (F2EMC) | |
| Fluorinated Ester 2 | trifluoroethyl methyl carbonate | 0.1 wt. %-5 wt. % |
| | (F3EMC) | |
| Fluorinated Ester 2 | trifluoroethyl methyl carbonate | >5 wt. %-25 wt. % |
| | (F3EMC) | |
| Fluorinated Ester 2 | trifluoroethyl methyl carbonate | >25 wt. %-95 wt. % |
| | (F3EMC) | |
| Fluorinated Ester 2 | bis(2,2,2-trifluoroethyl) carbonate | 0.1 wt. %-5 wt. % |
| | (TFEC) | |
| Fluorinated Ester 2 | bis(2,2,2-trifluoroethyl) carbonate | >5 wt. %-25 wt. % |
| | (TFEC) | |
| Fluorinated Ester 2 | bis(2,2,2-trifluoroethyl) carbonate | >25 wt. %-95 wt. % |
| | (TFEC) | |
| Fluorinated Ester 2 | 2,2-difluoroethyl ethyl carbonate | 0.1 wt. %-5 wt. % |
| | (DFDEC) | |
| Fluorinated Ester 2 | 2,2-difluoroethyl ethyl carbonate | >5 wt. %-25 wt. % |
| | (DFDEC) | |
| Fluorinated Ester 2 | 2,2-difluoroethyl ethyl carbonate | >25 wt. %-95 wt. % |
| | (DFDEC) | |
| Fluorinated Ester 2 | acetonitrile (AN) | 0.1 wt. %-5 wt. % |
| Fluorinated Ester 2 | acetonitrile (AN) | >5 wt. %-25 wt. % |
| Fluorinated Ester 2 | acetonitrile (AN) | >25 wt. %-95 wt. % |
| Fluorinated Ester 2 | fluoroacetonitrile | 0.1 wt. %-5 wt. % |
| Fluorinated Ester 2 | fluoroacetonitrile | >5 wt. %-25 wt. % |
| Fluorinated Ester 2 | fluoroacetonitrile | >25 wt. %-95 wt. % |
| Fluorinated Ester 2 | ethyl acetate (EA) | 0.1 wt. %-5 wt. % |
| Fluorinated Ester 2 | ethyl acetate (EA) | >5 wt. %-25 wt. % |
| Fluorinated Ester 2 | ethyl acetate (EA) | >25 wt. %-95 wt. % |
| Fluorinated Ester 2 | methyl acetate (MA) | 0.1 wt. %-5 wt. % |
| Fluorinated Ester 2 | methyl acetate (MA) | >5 wt. %-25 wt. % |
| Fluorinated Ester 2 | methyl acetate (MA) | >25 wt. %-95 wt. % |
| Fluorinated Ester 2 | methyl propanoate (MP) | 0.1 wt. %-5 wt. % |
| Fluorinated Ester 2 | methyl propanoate (MP) | >5 wt. %-25 wt. % |
| Fluorinated Ester 2 | methyl propanoate (MP) | >25 wt. %-95 wt. % |
| Fluorinated Ester 2 | methyl 3,3,3-trifluoropropanoate | 0.1 wt. %-5 wt. % |
| Fluorinated Ester 2 | methyl 3,3,3-trifluoropropanoate | >5 wt. %-25 wt. % |
| Fluorinated Ester 2 | methyl 3,3,3-trifluoropropanoate | >25 wt. %-95 wt. % |
| Fluorinated Ester 2 | ethyl 3,3,3-trifluoropropanoate | 0.1 wt. %-5 wt. % |
| Fluorinated Ester 2 | ethyl 3,3,3-trifluoropropanoate | >5 wt. %-25 wt. % |
| Fluorinated Ester 2 | ethyl 3,3,3-trifluoropropanoate | >25 wt. %-95 wt. % |
| Fluorinated Ester 2 | ethyl 2,2-difluoropropanoate | 0.1 wt. %-5 wt. % |
| Fluorinated Ester 2 | ethyl 2,2-difluoropropanoate | >5 wt. %-25 wt. % |
| Fluorinated Ester 2 | ethyl 2,2-difluoropropanoate | >25 wt. %-95 wt. % |
| Fluorinated Ester 2 | isopropyl trifluoroacetate | 0.1 wt. %-5 wt. % |
| Fluorinated Ester 2 | isopropyl trifluoroacetate | >5 wt. %-25 wt. % |
| Fluorinated Ester 2 | isopropyl trifluoroacetate | >25 wt. %-95 wt. % |
| Fluorinated Ester 2 | methyl 4,4,4-trifluorobutanoate | 0.1 wt. %-5 wt. % |
| Fluorinated Ester 2 | methyl 4,4,4-trifluorobutanoate | >5 wt. %-25 wt. % |
| Fluorinated Ester 2 | methyl 4,4,4-trifluorobutanoate | >25 wt. %-95 wt. % |
| Fluorinated Ester 2 | methyl difluoroacetate | 0.1 wt. %-5 wt. % |
| Fluorinated Ester 2 | methyl difluoroacetate | >5 wt. %-25 wt. % |
| Fluorinated Ester 2 | methyl difluoroacetate | >25 wt. %-95 wt. % |
| Fluorinated Ester 2 | 2,2,2-trifluoroethyl butanoate | 0.1 wt. %-5 wt. % |
| Fluorinated Ester 2 | 2,2,2-trifluoroethyl butanoate | >5 wt. %-25 wt. % |
| Fluorinated Ester 2 | 2,2,2-trifluoroethyl butanoate | >25 wt. %-95 wt. % |
| Fluorinated Ester 2 | methyl 2,2-difluoropropanoate | 0.1 wt. %-5 wt. % |
| Fluorinated Ester 2 | methyl 2,2-difluoropropanoate | >5 wt. %-25 wt. % |
| Fluorinated Ester 2 | methyl 2,2-difluoropropanoate | >25 wt. %-95 wt. % |
| Fluorinated Ester 2 | ethyl trifluoroacetate | 0.1 wt. %-5 wt. % |
| Fluorinated Ester 2 | ethyl trifluoroacetate | >5 wt. %-25 wt. % |
| Fluorinated Ester 2 | ethyl trifluoroacetate | >25 wt. %-95 wt. % |
| Fluorinated Ester 2 | 2,2-difluoroethyl trifluoroacetate | 0.1 wt. %-5 wt. % |
| Fluorinated Ester 2 | 2,2-difluoroethyl trifluoroacetate | >5 wt. %-25 wt. % |
| Fluorinated Ester 2 | 2,2-difluoroethyl trifluoroacetate | >25 wt. %-95 wt. % |
| Fluorinated Ester 2 | 2,2,2-trifluoroethyl acetate | 0.1 wt. %-5 wt. % |
| Fluorinated Ester 2 | 2,2,2-trifluoroethyl acetate | >5 wt. %-25 wt. % |
| Fluorinated Ester 2 | 2,2,2-trifluoroethyl acetate | >25 wt. %-95 wt. % |
| Fluorinated Ester 2 | ethyl 2,2-difluoroacetate | 0.1 wt. %-5 wt. % |
| Fluorinated Ester 2 | ethyl 2,2-difluoroacetate | >5 wt. %-25 wt. % |
| Fluorinated Ester 2 | ethyl 2,2-difluoroacetate | >25 wt. %-95 wt. % |
| Fluorinated Ester 2 | 2,2-difluoroethyl acetate | 0.1 wt. %-5 wt. % |
| Fluorinated Ester 2 | 2,2-difluoroethyl acetate | >5 wt. %-25 wt. % |
| Fluorinated Ester 2 | 2,2-difluoroethyl acetate | >25 wt. %-95 wt. % |
| Fluorinated Ester 2 | 2,2-difluoroethyl 3,3- | 0.1 wt. %-5 wt. % |
| | difluoropropanoate | |
| Fluorinated Ester 2 | 2,2-difluoroethyl 3,3- | >5 wt. %-25 wt. % |
| | difluoropropanoate | |
| Fluorinated Ester 2 | 2,2-difluoroethyl 3,3- | >25 wt. %-95 wt. % |
| | difluoropropanoate | |
| Fluorinated Ester 2 | 2,2-difluoroethyl 2,2-difluoroacetate | 0.1 wt. %-5 wt. % |
| Fluorinated Ester 2 | 2,2-difluoroethyl 2,2-difluoroacetate | >5 wt. %-25 wt. % |
| Fluorinated Ester 2 | 2,2-difluoroethyl 2,2-difluoroacetate | >25 wt. %-95 wt. % |
| Fluorinated Ester 2 | succinonitrile (SN) | 0.1 wt. %-5 wt. % |
| Fluorinated Ester 2 | succinonitrile (SN) | >5 wt. %-25 wt. % |
| Fluorinated Ester 2 | succinonitrile (SN) | >25 wt. %-95 wt. % |
| Fluorinated Ester 2 | propionitrile (PN) | 0.1 wt. %-5 wt. % |
| Fluorinated Ester 2 | propionitrile (PN) | >5 wt. %-25 wt. % |
| Fluorinated Ester 2 | propionitrile (PN) | >25 wt. %-95 wt. % |
| Fluorinated Ester 2 | butyronitrile (BN) | 0.1 wt. %-5 wt. % |
| Fluorinated Ester 2 | butyronitrile (BN) | >5 wt. %-25 wt. % |
| Fluorinated Ester 2 | butyronitrile (BN) | >25 wt. %-95 wt. % |
| Fluorinated Ester 2 | adiponitrile (ADN) | 0.1 wt. %-5 wt. % |
| Fluorinated Ester 2 | adiponitrile (ADN) | >5 wt. %-25 wt. % |
| Fluorinated Ester 2 | adiponitrile (ADN) | >25 wt. %-95 wt. % |
| Fluorinated Ester 2 | 1,3,6-hexanetricarbonitrile (HTCN) | 0.1 wt. %-5 wt. % |
| Fluorinated Ester 2 | 1,3,6-hexanetricarbonitrile (HTCN) | >5 wt. %-25 wt. % |
| Fluorinated Ester 2 | 1,3,6-hexanetricarbonitrile (HTCN) | >25 wt. %-95 wt. % |
| Fluorinated Ester 2 | trimethyl borate (TMB) | 0.1 wt. %-5 wt. % |
| Fluorinated Ester 2 | trimethyl borate (TMB) | >5 wt. %-25 wt. % |
| Fluorinated Ester 2 | trimethyl borate (TMB) | >25 wt. %-95 wt. % |
| Fluorinated Ester 2 | triphenyl borate (TPB) | 0.1 wt. %-5 wt. % |
| Fluorinated Ester 2 | triphenyl borate (TPB) | >5 wt. %-25 wt. % |
| Fluorinated Ester 2 | triphenyl borate (TPB) | >25 wt. %-95 wt. % |
| Fluorinated Ester 2 | triethyl borate (TEB) | 0.1 wt. %-5 wt. % |
| Fluorinated Ester 2 | triethyl borate (TEB) | >5 wt. %-25 wt. % |
| Fluorinated Ester 2 | triethyl borate (TEB) | >25 wt. %-95 wt. % |
| Fluorinated Ester 2 | tris(pentafluorophenyl)borane (TPFPB) | 0.1 wt. %-5 wt. % |
| Fluorinated Ester 2 | tris(pentafluorophenyl)borane (TPFPB) | >5 wt. %-25 wt. % |
| Fluorinated Ester 2 | tris(pentafluorophenyl)borane (TPFPB) | >25 wt. %-95 wt. % |
| Fluorinated Ester 2 | tris(trimethylsilyl)phosphate (TTSB) | 0.1 wt. %-5 wt. % |
| Fluorinated Ester 2 | tris(trimethylsilyl)phosphate (TTSB) | >5 wt. %-25 wt. % |
| Fluorinated Ester 2 | tris(trimethylsilyl)phosphate (TTSB) | >25 wt. %-95 wt. % |
| Fluorinated Ester 2 | tris(2,2,2-trifluoroethyl) borate (TTFEB) | 0.1 wt. %-5 wt. % |
| Fluorinated Ester 2 | tris(2,2,2-trifluoroethyl) borate (TTFEB) | >5 wt. %-25 wt. % |
| Fluorinated Ester 2 | tris(2,2,2-trifluoroethyl) borate (TTFEB) | >25 wt. %-95 wt. % |
| Fluorinated Ester 2 | trimethyl phosphate (TMP) | 0.1 wt. %-5 wt. % |
| Fluorinated Ester 2 | trimethyl phosphate (TMP) | >5 wt. %-25 wt. % |
| Fluorinated Ester 2 | trimethyl phosphate (TMP) | >25 wt. %-95 wt. % |
| Fluorinated Ester 2 | triethyl phosphate (TEP) | 0.1 wt. %-5 wt. % |
| Fluorinated Ester 2 | triethyl phosphate (TEP) | >5 wt. %-25 wt. % |
| Fluorinated Ester 2 | triethyl phosphate (TEP) | >25 wt. %-95 wt. % |
| Fluorinated Ester 2 | tris(trimethylsilyl)phosphate (TTSP) | 0.1 wt. %-5 wt. % |
| Fluorinated Ester 2 | tris(trimethylsilyl)phosphate (TTSP) | >5 wt. %-25 wt. % |
| Fluorinated Ester 2 | tris(trimethylsilyl)phosphate (TTSP) | >25 wt. %-95 wt. % |
| Fluorinated Ester 2 | tris(trimethylsilyl)phosphite (TTSPi) | 0.1 wt. %-5 wt. % |
| Fluorinated Ester 2 | tris(trimethylsilyl)phosphite (TTSPi) | >5 wt. %-25 wt. % |
| Fluorinated Ester 2 | tris(trimethylsilyl)phosphite (TTSPi) | >25 wt. %-95 wt. % |
| Fluorinated Ester 2 | tris(2,2,2-trifluoroethyl) phosphate | 0.1 wt. %-5 wt. % |
| | (TFEPa) | |
| Fluorinated Ester 2 | tris(2,2,2-trifluoroethyl) phosphate | >5 wt. %-25 wt. % |
| | (TFEPa) | |
| Fluorinated Ester 2 | tris(2,2,2-trifluoroethyl) phosphate | >25 wt. %-95 wt. % |
| | (TFEPa) | |
| Fluorinated Ester 2 | tris(2,2,2-trifluoroethyl) phosphite | 0.1 wt. %-5 wt. % |
| | (TFEPi) | |
| Fluorinated Ester 2 | tris(2,2,2-trifluoroethyl) phosphite | >5 wt. %-25 wt. % |
| | (TFEPi) | |
| Fluorinated Ester 2 | tris(2,2,2-trifluoroethyl) phosphite | >25 wt. %-95 wt. % |
| | (TFEPi) | |
| Fluorinated Ester 2 | (pentafluorophenyl)diphenyl phosphine | 0.1 wt. %-5 wt. % |
| | (PFPDPP) | |
| Fluorinated Ester 2 | (pentafluorophenyl)diphenyl phosphine | >5 wt. %-25 wt. % |
| | (PFPDPP) | |
| Fluorinated Ester 2 | (pentafluorophenyl)diphenyl phosphine | >25 wt. %-95 wt. % |
| | (PFPDPP) | |
| Fluorinated Ester 2 | tris(pentafluorophenyl) phosphine | 0.1 wt. %-5 wt. % |
| | (TPFPP) | |
| Fluorinated Ester 2 | tris(pentafluorophenyl) phosphine | >5 wt. %-25 wt. % |
| | (TPFPP) | |
| Fluorinated Ester 2 | tris(pentafluorophenyl) phosphine | >25 wt. %-95 wt. % |
| | (TPFPP) | |
| Fluorinated Ester 2 | 1,3,2-dioxathiolane-2,2-dioxide (DTD) | 0.1 wt. %-5 wt. % |
| Fluorinated Ester 2 | 1,3,2-dioxathiolane-2,2-dioxide (DTD) | >5 wt. %-25 wt. % |
| Fluorinated Ester 2 | 1,3,2-dioxathiolane-2,2-dioxide (DTD) | >25 wt. %-95 wt. % |
| Fluorinated Ester 2 | 1,3-propanesultone (PS) | 0.1 wt. %-5 wt. % |
| Fluorinated Ester 2 | 1,3-propanesultone (PS) | >5 wt. %-25 wt. % |
| Fluorinated Ester 2 | 1,3-propanesultone (PS) | >25 wt. %-95 wt. % |
| Fluorinated Ester 2 | prop-1-ene-1,3-sultone (PES) | 0.1 wt. %-5 wt. % |
| Fluorinated Ester 2 | prop-1-ene-1,3-sultone (PES) | >5 wt. %-25 wt. % |
| Fluorinated Ester 2 | prop-1-ene-1,3-sultone (PES) | >25 wt. %-95 wt. % |
| Fluorinated Ester 2 | propanediol cyclic sulfate (PCS) | 0.1 wt. %-5 wt. % |
| Fluorinated Ester 2 | propanediol cyclic sulfate (PCS) | >5 wt. %-25 wt. % |
| Fluorinated Ester 2 | propanediol cyclic sulfate (PCS) | >25 wt. %-95 wt. % |
| Fluorinated Ester 2 | 1,3,2-dioxathiolane 2-oxide | 0.1 wt. %-5 wt. % |
| Fluorinated Ester 2 | 1,3,2-dioxathiolane 2-oxide | >5 wt. %-25 wt. % |
| Fluorinated Ester 2 | 1,3,2-dioxathiolane 2-oxide | >25 wt. %-95 wt. % |
| Fluorinated Ester 2 | bis(2,2,2-trifluoroethyl) ether (BTFE) | 0.1 wt. %-5 wt. % |
| Fluorinated Ester 2 | bis(2,2,2-trifluoroethyl) ether (BTFE) | >5 wt. %-25 wt. % |
| Fluorinated Ester 2 | bis(2,2,2-trifluoroethyl) ether (BTFE) | >25 wt. %-95 wt. % |
| Fluorinated Ester 2 | 1,1,2,2-tetrafluoroethyl-2,2,3,3- | 0.1 wt. %-5 wt. % |
| | tetrafluoropropyl ether (TTE) | |
| Fluorinated Ester 2 | 1,1,2,2-tetrafluoroethyl-2,2,3,3- | >5 wt. %-25 wt. % |
| | tetrafluoropropyl ether (TTE) | |
| Fluorinated Ester 2 | 1,1,2,2-tetrafluoroethyl-2,2,3,3- | >25 wt. %-95 wt. % |
| | tetrafluoropropyl ether (TTE) | |
| Fluorinated Ester 2 | 1H,1H,5H-octafluoropentyl-1,1,2,2- | 0.1 wt. %-5 wt. % |
| | tetrafluoroethylether (OTE) | |
| Fluorinated Ester 2 | 1H,1H,5H-octafluoropentyl-1,1,2,2- | >5 wt. %-25 wt. % |
| | tetrafluoroethylether (OTE) | |
| Fluorinated Ester 2 | 1H,1H,5H-octafluoropentyl-1,1,2,2- | >25 wt. %-95 wt. % |
| | tetrafluoroethylether (OTE) | |
| Fluorinated Ester 2 | 2,2,2-trifluoroethyl 1,1,2,2- | 0.1 wt. %-5 wt. % |
| | tetrafluoroethyl ether (HFE) | |
| Fluorinated Ester 2 | 2,2,2-trifluoroethyl 1,1,2,2- | >5 wt. %-25 wt. % |
| | tetrafluoroethyl ether (HFE) | |
| Fluorinated Ester 2 | 2,2,2-trifluoroethyl 1,1,2,2- | >25 wt. %-95 wt. % |
| | tetrafluoroethyl ether (HFE) | |
| Fluorinated Ester 2 | tetrahydrofuran (THF) | 0.1 wt. %-5 wt. % |
| Fluorinated Ester 2 | tetrahydrofuran (THF) | >5 wt. %-25 wt. % |
| Fluorinated Ester 2 | tetrahydrofuran (THF) | >25 wt. %-95 wt. % |
| Fluorinated Ester 2 | 1,3-dioxolane (DOL) | 0.1 wt. %-5 wt. % |
| Fluorinated Ester 2 | 1,3-dioxolane (DOL) | >5 wt. %-25 wt. % |
| Fluorinated Ester 2 | 1,3-dioxolane (DOL) | >25 wt. %-95 wt. % |
| Fluorinated Ester 2 | 1,2-dimethoxyethane (DME) | 0.1 wt. %-5 wt. % |
| Fluorinated Ester 2 | 1,2-dimethoxyethane (DME) | >5 wt. %-25 wt. % |
| Fluorinated Ester 2 | 1,2-dimethoxyethane (DME) | >25 wt. %-95 wt. % |
| Fluorinated Ester 2 | diethylene glycol dimethyl ether | 0.1 wt. %-5 wt. % |
| | (DEGDME) | |
| Fluorinated Ester 2 | diethylene glycol dimethyl ether | >5 wt. %-25 wt. % |
| | (DEGDME) | |
| Fluorinated Ester 2 | diethylene glycol dimethyl ether | >25 wt. %-95 wt. % |
| | (DEGDME) | |
| Fluorinated Ester 2 | tetraethylene glycol dimethyl ether | 0.1 wt. %-5 wt. % |
| | (TEGDME) | |
| Fluorinated Ester 2 | tetraethylene glycol dimethyl ether | >5 wt. %-25 wt. % |
| | (TEGDME) | |
| Fluorinated Ester 2 | tetraethylene glycol dimethyl ether | >25 wt. %-95 wt. % |
| | (TEGDME) | |
| Fluorinated Ester 2 | succinic anhydride (SA) | 0.1 wt. %-5 wt. % |
| Fluorinated Ester 2 | succinic anhydride (SA) | >5 wt. %-25 wt. % |
| Fluorinated Ester 2 | succinic anhydride (SA) | >25 wt. %-95 wt. % |
| Fluorinated Ester 2 | butyric anhydride (BA) | 0.1 wt. %-5 wt. % |
| Fluorinated Ester 2 | butyric anhydride (BA) | >5 wt. %-25 wt. % |
| Fluorinated Ester 2 | butyric anhydride (BA) | >25 wt. %-95 wt. % |
| Fluorinated Ester 2 | 1,4-dioxane (DOX) | 0.1 wt. %-5 wt. % |
| Fluorinated Ester 2 | 1,4-dioxane (DOX) | >5 wt. %-25 wt. % |
| Fluorinated Ester 2 | 1,4-dioxane (DOX) | >25 wt. %-95 wt. % |
| Fluorinated Ester 2 | tetravinyl silane (TVSI) | 0.1 wt. %-5 wt. % |
| Fluorinated Ester 2 | tetravinyl silane (TVSI) | >5 wt. %-25 wt. % |
| Fluorinated Ester 2 | tetravinyl silane (TVSI) | >25 wt. %-95 wt. % |
| Fluorinated Ester 2 | 1,4-butane sultone (BS) | 0.1 wt. %-5 wt. % |
| Fluorinated Ester 2 | 1,4-butane sultone (BS) | >5 wt. %-25 wt. % |
| Fluorinated Ester 2 | 1,4-butane sultone (BS) | >25 wt. %-95 wt. % |
| Fluorinated Ester 2 | dimethyl sulfoxide (DMSO) | 0.1 wt. %-5 wt. % |
| Fluorinated Ester 2 | dimethyl sulfoxide (DMSO) | >5 wt. %-25 wt. % |
| Fluorinated Ester 2 | dimethyl sulfoxide (DMSO) | >25 wt. %-95 wt. % |
| Fluorinated Ester 2 | methylene methanedisulfonate | 0.1 wt. %-5 wt. % |
| | (MMDS) | |
| Fluorinated Ester 2 | methylene methanedisulfonate | >5 wt. %-25 wt. % |
| | (MMDS) | |
| Fluorinated Ester 2 | methylene methanedisulfonate | >25 wt. %-95 wt. % |
| | (MMDS) | |
| Fluorinated Ester 2 | N,N-dimethylformamide (DMF) | 0.1 wt. %-5 wt. % |
| Fluorinated Ester 2 | N,N-dimethylformamide (DMF) | >5 wt. %-25 wt. % |
| Fluorinated Ester 2 | N,N-dimethylformamide (DMF) | >25 wt. %-95 wt. % |
| Fluorinated Ester 2 | gamma-butyrolactone (BL) | 0.1 wt. %-5 wt. % |
| Fluorinated Ester 2 | gamma-butyrolactone (BL) | >5 wt. %-25 wt. % |
| Fluorinated Ester 2 | gamma-butyrolactone (BL) | >25 wt. %-95 wt. % |

TABLE 9C

| | | Concentration of |
|---|---|---|
| Fluorinated Ester | Solvent | Fluorinated Ester 3 |
| Fluorinated Ester 3 | fluoroethylene carbonate (FEC) | 0.1 wt. %-5 wt. % |
| Fluorinated Ester 3 | fluoroethylene carbonate (FEC) | >5 wt. %-25 wt. % |
| Fluorinated Ester 3 | fluoroethylene carbonate (FEC) | >25 wt. %-95 wt. % |
| Fluorinated Ester 3 | propylene carbonate (PC) | 0.1 wt. %-5 wt. % |
| Fluorinated Ester 3 | propylene carbonate (PC) | >5 wt. %-25 wt. % |
| Fluorinated Ester 3 | propylene carbonate (PC) | >25 wt. %-95 wt. % |
| Fluorinated Ester 3 | dimethyl carbonate (DMC) | 0.1 wt. %-5 wt. % |
| Fluorinated Ester 3 | dimethyl carbonate (DMC) | >5 wt. %-25 wt. % |
| Fluorinated Ester 3 | dimethyl carbonate (DMC) | >25 wt. %-95 wt. % |
| Fluorinated Ester 3 | diethyl carbonate (DEC) | 0.1 wt. %-5 wt. % |
| Fluorinated Ester 3 | diethyl carbonate (DEC) | >5 wt. %-25 wt. % |
| Fluorinated Ester 3 | diethyl carbonate (DEC) | >25 wt. %-95 wt. % |
| Fluorinated Ester 3 | ethyl methyl carbonate (EMC) | 0.1 wt. %-5 wt. % |
| Fluorinated Ester 3 | ethyl methyl carbonate (EMC) | >5 wt. %-25 wt. % |
| Fluorinated Ester 3 | ethyl methyl carbonate (EMC) | >25 wt. %-95 wt. % |
| Fluorinated Ester 3 | vinyl carbonate (VC) | 0.1 wt. %-5 wt. % |
| Fluorinated Ester 3 | vinyl carbonate (VC) | >5 wt. %-25 wt. % |
| Fluorinated Ester 3 | vinyl carbonate (VC) | >25 wt. %-95 wt. % |
| Fluorinated Ester 3 | vinyl ethylene carbonate (VEC) | 0.1 wt. %-5 wt. % |
| Fluorinated Ester 3 | vinyl ethylene carbonate (VEC) | >5 wt. %-25 wt. % |
| Fluorinated Ester 3 | vinyl ethylene carbonate (VEC) | >25 wt. %-95 wt. % |
| Fluorinated Ester 3 | ethylene carbonate (EC) | 0.1 wt. %-5 wt. % |
| Fluorinated Ester 3 | ethylene carbonate (EC) | >5 wt. %-25 wt. % |
| Fluorinated Ester 3 | ethylene carbonate (EC) | >25 wt. %-95 wt. % |
| Fluorinated Ester 3 | difluoroethylene carbonate (DFEC) | 0.1 wt. %-5 wt. % |
| Fluorinated Ester 3 | difluoroethylene carbonate (DFEC) | >5 wt. %-25 wt. % |
| Fluorinated Ester 3 | difluoroethylene carbonate (DFEC) | >25 wt. %-95 wt. % |
| Fluorinated Ester 3 | 3,3,3-trifluoropropylene carbonate | 0.1 wt. %-5 wt. % |
| | (TFPC) | |
| Fluorinated Ester 3 | 3,3,3-trifluoropropylene carbonate | >5 wt. %-25 wt. % |
| | (TFPC) | |
| Fluorinated Ester 3 | 3,3,3-trifluoropropylene carbonate | >25 wt. %-95 wt. % |
| | (TFPC) | |
| Fluorinated Ester 3 | monofluoroethyl methyl carbonate | 0.1 wt. %-5 wt. % |
| | (F1EMC) | |
| Fluorinated Ester 3 | monofluoroethyl methyl carbonate | >5 wt. %-25 wt. % |
| | (F1EMC) | |
| Fluorinated Ester 3 | monofluoroethyl methyl carbonate | >25 wt. %-95 wt. % |
| | (F1EMC) | |
| Fluorinated Ester 3 | difluoroethyl methyl carbonate | 0.1 wt. %-5 wt. % |
| | (F2EMC) | |
| Fluorinated Ester 3 | difluoroethyl methyl carbonate | >5 wt. %-25 wt. % |
| | (F2EMC) | |
| Fluorinated Ester 3 | difluoroethyl methyl carbonate | >25 wt. %-95 wt. % |
| | (F2EMC) | |
| Fluorinated Ester 3 | trifluoroethyl methyl carbonate | 0.1 wt. %-5 wt. % |
| | (F3EMC) | |
| Fluorinated Ester 3 | trifluoroethyl methyl carbonate | >5 wt. %-25 wt. % |
| | (F3EMC) | |
| Fluorinated Ester 3 | trifluoroethyl methyl carbonate | >25 wt. %-95 wt. % |
| | (F3EMC) | |
| Fluorinated Ester 3 | bis(2,2,2-trifluoroethyl) carbonate | 0.1 wt. %-5 wt. % |
| | (TFEC) | |
| Fluorinated Ester 3 | bis(2,2,2-trifluoroethyl) carbonate | >5 wt. %-25 wt. % |
| | (TFEC) | |
| Fluorinated Ester 3 | bis(2,2,2-trifluoroethyl) carbonate | >25 wt. %-95 wt. % |
| | (TFEC) | |
| Fluorinated Ester 3 | 2,2-difluoroethyl ethyl carbonate | 0.1 wt. %-5 wt. % |
| | (DFDEC) | |
| Fluorinated Ester 3 | 2,2-difluoroethyl ethyl carbonate | >5 wt. %-25 wt. % |
| | (DFDEC) | |
| Fluorinated Ester 3 | 2,2-difluoroethyl ethyl carbonate | >25 wt. %-95 wt. % |
| | (DFDEC) | |
| Fluorinated Ester 3 | acetonitrile (AN) | 0.1 wt. %-5 wt. % |
| Fluorinated Ester 3 | acetonitrile (AN) | >5 wt. %-25 wt. % |
| Fluorinated Ester 3 | acetonitrile (AN) | >25 wt. %-95 wt. % |
| Fluorinated Ester 3 | fluoroacetonitrile | 0.1 wt. %-5 wt. % |
| Fluorinated Ester 3 | fluoroacetonitrile | >5 wt. %-25 wt. % |
| Fluorinated Ester 3 | fluoroacetonitrile | >25 wt. %-95 wt. % |
| Fluorinated Ester 3 | ethyl acetate (EA) | 0.1 wt. %-5 wt. % |
| Fluorinated Ester 3 | ethyl acetate (EA) | >5 wt. %-25 wt. % |
| Fluorinated Ester 3 | ethyl acetate (EA) | >25 wt. %-95 wt. % |
| Fluorinated Ester 3 | methyl acetate (MA) | 0.1 wt. %-5 wt. % |
| Fluorinated Ester 3 | methyl acetate (MA) | >5 wt. %-25 wt. % |
| Fluorinated Ester 3 | methyl acetate (MA) | >25 wt. %-95 wt. % |
| Fluorinated Ester 3 | methyl propanoate (MP) | 0.1 wt. %-5 wt. % |
| Fluorinated Ester 3 | methyl propanoate (MP) | >5 wt. %-25 wt. % |
| Fluorinated Ester 3 | methyl propanoate (MP) | >25 wt. %-95 wt. % |
| Fluorinated Ester 3 | methyl 3,3,3-trifluoropropanoate | 0.1 wt. %-5 wt. % |
| Fluorinated Ester 3 | methyl 3,3,3-trifluoropropanoate | >5 wt. %-25 wt. % |
| Fluorinated Ester 3 | methyl 3,3,3-trifluoropropanoate | >25 wt. %-95 wt. % |
| Fluorinated Ester 3 | ethyl 3,3,3-trifluoropropanoate | 0.1 wt. %-5 wt. % |
| Fluorinated Ester 3 | ethyl 3,3,3-trifluoropropanoate | >5 wt. %-25 wt. % |
| Fluorinated Ester 3 | ethyl 3,3,3-trifluoropropanoate | >25 wt. %-95 wt. % |
| Fluorinated Ester 3 | ethyl 2,2-difluoropropanoate | 0.1 wt. %-5 wt. % |
| Fluorinated Ester 3 | ethyl 2,2-difluoropropanoate | >5 wt. %-25 wt. % |
| Fluorinated Ester 3 | ethyl 2,2-difluoropropanoate | >25 wt. %-95 wt. % |
| Fluorinated Ester 3 | isopropyl trifluoroacetate | 0.1 wt. %-5 wt. % |
| Fluorinated Ester 3 | isopropyl trifluoroacetate | >5 wt. %-25 wt. % |
| Fluorinated Ester 3 | isopropyl trifluoroacetate | >25 wt. %-95 wt. % |
| Fluorinated Ester 3 | methyl 4,4,4-trifluorobutanoate | 0.1 wt. %-5 wt. % |
| Fluorinated Ester 3 | methyl 4,4,4-trifluorobutanoate | >5 wt. %-25 wt. % |
| Fluorinated Ester 3 | methyl 4,4,4-trifluorobutanoate | >25 wt. %-95 wt. % |
| Fluorinated Ester 3 | methyl difluoroacetate | 0.1 wt. %-5 wt. % |
| Fluorinated Ester 3 | methyl difluoroacetate | >5 wt. %-25 wt. % |
| Fluorinated Ester 3 | methyl difluoroacetate | >25 wt. %-95 wt. % |
| Fluorinated Ester 3 | 2,2,2-trifluoroethyl butanoate | 0.1 wt. %-5 wt. % |
| Fluorinated Ester 3 | 2,2,2-trifluoroethyl butanoate | >5 wt. %-25 wt. % |
| Fluorinated Ester 3 | 2,2,2-trifluoroethyl butanoate | >25 wt. %-95 wt. % |
| Fluorinated Ester 3 | methyl 2,2-difluoropropanoate | 0.1 wt. %-5 wt. % |
| Fluorinated Ester 3 | methyl 2,2-difluoropropanoate | >5 wt. %-25 wt. % |
| Fluorinated Ester 3 | methyl 2,2-difluoropropanoate | >25 wt. %-95 wt. % |
| Fluorinated Ester 3 | ethyl trifluoroacetate | 0.1 wt. %-5 wt. % |
| Fluorinated Ester 3 | ethyl trifluoroacetate | >5 wt. %-25 wt. % |
| Fluorinated Ester 3 | ethyl trifluoroacetate | >25 wt. %-95 wt. % |
| Fluorinated Ester 3 | 2,2-difluoroethyl trifluoroacetate | 0.1 wt. %-5 wt. % |
| Fluorinated Ester 3 | 2,2-difluoroethyl trifluoroacetate | >5 wt. %-25 wt. % |
| Fluorinated Ester 3 | 2,2-difluoroethyl trifluoroacetate | >25 wt. %-95 wt. % |
| Fluorinated Ester 3 | 2,2,2-trifluoroethyl acetate | 0.1 wt. %-5 wt. % |
| Fluorinated Ester 3 | 2,2,2-trifluoroethyl acetate | >5 wt. %-25 wt. % |
| Fluorinated Ester 3 | 2,2,2-trifluoroethyl acetate | >25 wt. %-95 wt. % |
| Fluorinated Ester 3 | ethyl 2,2-difluoroacetate | 0.1 wt. %-5 wt. % |
| Fluorinated Ester 3 | ethyl 2,2-difluoroacetate | >5 wt. %-25 wt. % |
| Fluorinated Ester 3 | ethyl 2,2-difluoroacetate | >25 wt. %-95 wt. % |
| Fluorinated Ester 3 | 2,2-difluoroethyl acetate | 0.1 wt. %-5 wt. % |
| Fluorinated Ester 3 | 2,2-difluoroethyl acetate | >5 wt. %-25 wt. % |
| Fluorinated Ester 3 | 2,2-difluoroethyl acetate | >25 wt. %-95 wt. % |
| Fluorinated Ester 3 | 2,2-difluoroethyl 3,3- | 0.1 wt. %-5 wt. % |
| | difluoropropanoate | |
| Fluorinated Ester 3 | 2,2-difluoroethyl 3,3- | >5 wt. %-25 wt. % |
| | difluoropropanoate | |
| Fluorinated Ester 3 | 2,2-difluoroethyl 3,3- | >25 wt. %-95 wt. % |
| | difluoropropanoate | |
| Fluorinated Ester 3 | 2,2-difluoroethyl 2,2-difluoroacetate | 0.1 wt. %-5 wt. % |
| Fluorinated Ester 3 | 2,2-difluoroethyl 2,2-difluoroacetate | >5 wt. %-25 wt. % |
| Fluorinated Ester 3 | 2,2-difluoroethyl 2,2-difluoroacetate | >25 wt. %-95 wt. % |
| Fluorinated Ester 3 | succinonitrile (SN) | 0.1 wt. %-5 wt. % |
| Fluorinated Ester 3 | succinonitrile (SN) | >5 wt. %-25 wt. % |
| Fluorinated Ester 3 | succinonitrile (SN) | >25 wt. %-95 wt. % |
| Fluorinated Ester 3 | propionitrile (PN) | 0.1 wt. %-5 wt. % |
| Fluorinated Ester 3 | propionitrile (PN) | >5 wt. %-25 wt. % |
| Fluorinated Ester 3 | propionitrile (PN) | >25 wt. %-95 wt. % |
| Fluorinated Ester 3 | butyronitrile (BN) | 0.1 wt. %-5 wt. % |
| Fluorinated Ester 3 | butyronitrile (BN) | >5 wt. %-25 wt. % |
| Fluorinated Ester 3 | butyronitrile (BN) | >25 wt. %-95 wt. % |
| Fluorinated Ester 3 | adiponitrile (ADN) | 0.1 wt. %-5 wt. % |
| Fluorinated Ester 3 | adiponitrile (ADN) | >5 wt. %-25 wt. % |
| Fluorinated Ester 3 | adiponitrile (ADN) | >25 wt. %-95 wt. % |
| Fluorinated Ester 3 | 1,3,6-hexanetricarbonitrile (HTCN) | 0.1 wt. %-5 wt. % |
| Fluorinated Ester 3 | 1,3,6-hexanetricarbonitrile (HTCN) | >5 wt. %-25 wt. % |
| Fluorinated Ester 3 | 1,3,6-hexanetricarbonitrile (HTCN) | >25 wt. %-95 wt. % |
| Fluorinated Ester 3 | trimethyl borate (TMB) | 0.1 wt. %-5 wt. % |
| Fluorinated Ester 3 | trimethyl borate (TMB) | >5 wt. %-25 wt. % |
| Fluorinated Ester 3 | trimethyl borate (TMB) | >25 wt. %-95 wt. % |
| Fluorinated Ester 3 | triphenyl borate (TPB) | 0.1 wt. %-5 wt. % |
| Fluorinated Ester 3 | triphenyl borate (TPB) | >5 wt. %-25 wt. % |
| Fluorinated Ester 3 | triphenyl borate (TPB) | >25 wt. %-95 wt. % |
| Fluorinated Ester 3 | triethyl borate (TEB) | 0.1 wt. %-5 wt. % |
| Fluorinated Ester 3 | triethyl borate (TEB) | >5 wt. %-25 wt. % |
| Fluorinated Ester 3 | triethyl borate (TEB) | >25 wt. %-95 wt. % |
| Fluorinated Ester 3 | tris(pentafluorophenyl)borane (TPFPB) | 0.1 wt. %-5 wt. % |
| Fluorinated Ester 3 | tris(pentafluorophenyl)borane (TPFPB) | >5 wt. %-25 wt. % |
| Fluorinated Ester 3 | tris(pentafluorophenyl)borane (TPFPB) | >25 wt. %-95 wt. % |
| Fluorinated Ester 3 | tris(trimethylsilyl)phosphate (TTSB) | 0.1 wt. %-5 wt. % |
| Fluorinated Ester 3 | tris(trimethylsilyl)phosphate (TTSB) | >5 wt. %-25 wt. % |
| Fluorinated Ester 3 | tris(trimethylsilyl)phosphate (TTSB) | >25 wt. %-95 wt. % |
| Fluorinated Ester 3 | tris(2,2,2-trifluoroethyl) borate (TTFEB) | 0.1 wt. %-5 wt. % |
| Fluorinated Ester 3 | tris(2,2,2-trifluoroethyl) borate (TTFEB) | >5 wt. %-25 wt. % |
| Fluorinated Ester 3 | tris(2,2,2-trifluoroethyl) borate (TTFEB) | >25 wt. %-95 wt. % |
| Fluorinated Ester 3 | trimethyl phosphate (TMP) | 0.1 wt. %-5 wt. % |
| Fluorinated Ester 3 | trimethyl phosphate (TMP) | >5 wt. %-25 wt. % |
| Fluorinated Ester 3 | trimethyl phosphate (TMP) | >25 wt. %-95 wt. % |
| Fluorinated Ester 3 | triethyl phosphate (TEP) | 0.1 wt. %-5 wt. % |
| Fluorinated Ester 3 | triethyl phosphate (TEP) | >5 wt. %-25 wt. % |
| Fluorinated Ester 3 | triethyl phosphate (TEP) | >25 wt. %-95 wt. % |
| Fluorinated Ester 3 | tris(trimethylsilyl)phosphate (TTSP) | 0.1 wt. %-5 wt. % |
| Fluorinated Ester 3 | tris(trimethylsilyl)phosphate (TTSP) | >5 wt. %-25 wt. % |
| Fluorinated Ester 3 | tris(trimethylsilyl)phosphate (TTSP) | >25 wt. %-95 wt. % |
| Fluorinated Ester 3 | tris(trimethylsilyl)phosphite (TTSPi) | 0.1 wt. %-5 wt. % |
| Fluorinated Ester 3 | tris(trimethylsilyl)phosphite (TTSPi) | >5 wt. %-25 wt. % |
| Fluorinated Ester 3 | tris(trimethylsilyl)phosphite (TTSPi) | >25 wt. %-95 wt. % |
| Fluorinated Ester 3 | tris(2,2,2-trifluoroethyl) phosphate | 0.1 wt. %-5 wt. % |
| | (TFEPa) | |
| Fluorinated Ester 3 | tris(2,2,2-trifluoroethyl) phosphate | >5 wt. %-25 wt. % |
| | (TFEPa) | |
| Fluorinated Ester 3 | tris(2,2,2-trifluoroethyl) phosphate | >25 wt. %-95 wt. % |
| | (TFEPa) | |
| Fluorinated Ester 3 | tris(2,2,2-trifluoroethyl) phosphite | 0.1 wt. %-5 wt. % |
| | (TFEPi) | |
| Fluorinated Ester 3 | tris(2,2,2-trifluoroethyl) phosphite | >5 wt. %-25 wt. % |
| | (TFEPi) | |
| Fluorinated Ester 3 | tris(2,2,2-trifluoroethyl) phosphite | >25 wt. %-95 wt. % |
| | (TFEPi) | |
| Fluorinated Ester 3 | (pentafluorophenyl)diphenyl phosphine | 0.1 wt. %-5 wt. % |
| | (PFPDPP) | |
| Fluorinated Ester 3 | (pentafluorophenyl)diphenyl phosphine | >5 wt. %-25 wt. % |
| | (PFPDPP) | |
| Fluorinated Ester 3 | (pentafluorophenyl)diphenyl phosphine | >25 wt. %-95 wt. % |
| | (PFPDPP) | |
| Fluorinated Ester 3 | tris(pentafluorophenyl) phosphine | 0.1 wt. %-5 wt. % |
| | (TPFPP) | |
| Fluorinated Ester 3 | tris(pentafluorophenyl) phosphine | >5 wt. %-25 wt. % |
| | (TPFPP) | |
| Fluorinated Ester 3 | tris(pentafluorophenyl) phosphine | >25 wt. %-95 wt. % |
| | (TPFPP) | |
| Fluorinated Ester 3 | 1,3,2-dioxathiolane-2,2-dioxide (DTD) | 0.1 wt. %-5 wt. % |
| Fluorinated Ester 3 | 1,3,2-dioxathiolane-2,2-dioxide (DTD) | >5 wt. %-25 wt. % |
| Fluorinated Ester 3 | 1,3,2-dioxathiolane-2,2-dioxide (DTD) | >25 wt. %-95 wt. % |
| Fluorinated Ester 3 | 1,3-propanesultone (PS) | 0.1 wt. %-5 wt. % |
| Fluorinated Ester 3 | 1,3-propanesultone (PS) | >5 wt. %-25 wt. % |
| Fluorinated Ester 3 | 1,3-propanesultone (PS) | >25 wt. %-95 wt. % |
| Fluorinated Ester 3 | prop-1-ene-1,3-sultone (PES) | 0.1 wt. %-5 wt. % |
| Fluorinated Ester 3 | prop-1-ene-1,3-sultone (PES) | >5 wt. %-25 wt. % |
| Fluorinated Ester 3 | prop-1-ene-1,3-sultone (PES) | >25 wt. %-95 wt. % |
| Fluorinated Ester 3 | propanediol cyclic sulfate (PCS) | 0.1 wt. %-5 wt. % |
| Fluorinated Ester 3 | propanediol cyclic sulfate (PCS) | >5 wt. %-25 wt. % |
| Fluorinated Ester 3 | propanediol cyclic sulfate (PCS) | >25 wt. %-95 wt. % |
| Fluorinated Ester 3 | 1,3,2-dioxathiolane 2-oxide | 0.1 wt. %-5 wt. % |
| Fluorinated Ester 3 | 1,3,2-dioxathiolane 2-oxide | >5 wt. %-25 wt. % |
| Fluorinated Ester 3 | 1,3,2-dioxathiolane 2-oxide | >25 wt. %-95 wt. % |
| Fluorinated Ester 3 | bis(2,2,2-trifluoroethyl) ether (BTFE) | 0.1 wt. %-5 wt. % |
| Fluorinated Ester 3 | bis(2,2,2-trifluoroethyl) ether (BTFE) | >5 wt. %-25 wt. % |
| Fluorinated Ester 3 | bis(2,2,2-trifluoroethyl) ether (BTFE) | >25 wt. %-95 wt. % |
| Fluorinated Ester 3 | 1,1,2,2-tetrafluoroethyl-2,2,3,3- | 0.1 wt. %-5 wt. % |
| | tetrafluoropropyl ether (TTE) | |
| Fluorinated Ester 3 | 1,1,2,2-tetrafluoroethyl-2,2,3,3- | >5 wt. %-25 wt. % |
| | tetrafluoropropyl ether (TTE) | |
| Fluorinated Ester 3 | 1,1,2,2-tetrafluoroethyl-2,2,3,3- | >25 wt. %-95 wt. % |
| | tetrafluoropropyl ether (TTE) | |
| Fluorinated Ester 3 | 1H,1H,5H-octafluoropentyl-1,1,2,2- | 0.1 wt. %-5 wt. % |
| | tetrafluoroethylether (OTE) | |
| Fluorinated Ester 3 | 1H,1H,5H-octafluoropentyl-1,1,2,2- | >5 wt. %-25 wt. % |
| | tetrafluoroethylether (OTE) | |
| Fluorinated Ester 3 | 1H,1H,5H-octafluoropentyl-1,1,2,2- | >25 wt. %-95 wt. % |
| | tetrafluoroethylether (OTE) | |
| Fluorinated Ester 3 | 2,2,2-trifluoroethyl 1,1,2,2- | 0.1 wt. %-5 wt. % |
| | tetrafluoroethyl ether (HFE) | |
| Fluorinated Ester 3 | 2,2,2-trifluoroethyl 1,1,2,2- | >5 wt. %-25 wt. % |
| | tetrafluoroethyl ether (HFE) | |
| Fluorinated Ester 3 | 2,2,2-trifluoroethyl 1,1,2,2- | >25 wt. %-95 wt. % |
| | tetrafluoroethyl ether (HFE) | |
| Fluorinated Ester 3 | tetrahydrofuran (THF) | 0.1 wt. %-5 wt. % |
| Fluorinated Ester 3 | tetrahydrofuran (THF) | >5 wt. %-25 wt. % |
| Fluorinated Ester 3 | tetrahydrofuran (THF) | >25 wt. %-95 wt. % |
| Fluorinated Ester 3 | 1,3-dioxolane (DOL) | 0.1 wt. %-5 wt. % |
| Fluorinated Ester 3 | 1,3-dioxolane (DOL) | >5 wt. %-25 wt. % |
| Fluorinated Ester 3 | 1,3-dioxolane (DOL) | >25 wt. %-95 wt. % |
| Fluorinated Ester 3 | 1,2-dimethoxyethane (DME) | 0.1 wt. %-5 wt. % |
| Fluorinated Ester 3 | 1,2-dimethoxyethane (DME) | >5 wt. %-25 wt. % |
| Fluorinated Ester 3 | 1,2-dimethoxyethane (DME) | >25 wt. %-95 wt. % |
| Fluorinated Ester 3 | diethylene glycol dimethyl ether | 0.1 wt. %-5 wt. % |
| | (DEGDME) | |
| Fluorinated Ester 3 | diethylene glycol dimethyl ether | >5 wt. %-25 wt. % |
| | (DEGDME) | |
| Fluorinated Ester 3 | diethylene glycol dimethyl ether | >25 wt. %-95 wt. % |
| | (DEGDME) | |
| Fluorinated Ester 3 | tetraethylene glycol dimethyl ether | 0.1 wt. %-5 wt. % |
| | (TEGDME) | |
| Fluorinated Ester 3 | tetraethylene glycol dimethyl ether | >5 wt. %-25 wt. % |
| | (TEGDME) | |
| Fluorinated Ester 3 | tetraethylene glycol dimethyl ether | >25 wt. %-95 wt. % |
| | (TEGDME) | |
| Fluorinated Ester 3 | succinic anhydride (SA) | 0.1 wt. %-5 wt. % |
| Fluorinated Ester 3 | succinic anhydride (SA) | >5 wt. %-25 wt. % |
| Fluorinated Ester 3 | succinic anhydride (SA) | >25 wt. %-95 wt. % |
| Fluorinated Ester 3 | butyric anhydride (BA) | 0.1 wt. %-5 wt. % |
| Fluorinated Ester 3 | butyric anhydride (BA) | >5 wt. %-25 wt. % |
| Fluorinated Ester 3 | butyric anhydride (BA) | >25 wt. %-95 wt. % |
| Fluorinated Ester 3 | 1,4-dioxane (DOX) | 0.1 wt. %-5 wt. % |
| Fluorinated Ester 3 | 1,4-dioxane (DOX) | >5 wt. %-25 wt. % |
| Fluorinated Ester 3 | 1,4-dioxane (DOX) | >25 wt. %-95 wt. % |
| Fluorinated Ester 3 | tetravinyl silane (TVSI) | 0.1 wt. %-5 wt. % |
| Fluorinated Ester 3 | tetravinyl silane (TVSI) | >5 wt. %-25 wt. % |
| Fluorinated Ester 3 | tetravinyl silane (TVSI) | >25 wt. %-95 wt. % |
| Fluorinated Ester 3 | 1,4-butane sultone (BS) | 0.1 wt. %-5 wt. % |
| Fluorinated Ester 3 | 1,4-butane sultone (BS) | >5 wt. %-25 wt. % |
| Fluorinated Ester 3 | 1,4-butane sultone (BS) | >25 wt. %-95 wt. % |
| Fluorinated Ester 3 | dimethyl sulfoxide (DMSO) | 0.1 wt. %-5 wt. % |
| Fluorinated Ester 3 | dimethyl sulfoxide (DMSO) | >5 wt. %-25 wt. % |
| Fluorinated Ester 3 | dimethyl sulfoxide (DMSO) | >25 wt. %-95 wt. % |
| Fluorinated Ester 3 | methylene methanedisulfonate | 0.1 wt. %-5 wt. % |
| | (MMDS) | |
| Fluorinated Ester 3 | methylene methanedisulfonate | >5 wt. %-25 wt. % |
| | (MMDS) | |
| Fluorinated Ester 3 | methylene methanedisulfonate | >25 wt. %-95 wt. % |
| | (MMDS) | |
| Fluorinated Ester 3 | N,N-dimethylformamide (DMF) | 0.1 wt. %-5 wt. % |
| Fluorinated Ester 3 | N,N-dimethylformamide (DMF) | >5 wt. %-25 wt. % |
| Fluorinated Ester 3 | N,N-dimethylformamide (DMF) | >25 wt. %-95 wt. % |
| Fluorinated Ester 3 | gamma-butyrolactone (BL) | 0.1 wt. %-5 wt. % |
| Fluorinated Ester 3 | gamma-butyrolactone (BL) | >5 wt. %-25 wt. % |
| Fluorinated Ester 3 | gamma-butyrolactone (BL) | >25 wt. %-95 wt. % |

Tables 9D-9F includes such solvents that are not one of Fluorinated Esters 1-3 that can be included in the electrolyte. The amount of supplemental solvent in the electrolyte can be from about 0.5 volume percent (vol. %) to about 99.0 vol. %, based on a total volume of the electrolyte. Fluorinated Esters 1-3 can be present in an amount of from 0.1 vol. % to 5 vol. % (e.g., as an additive), from greater than 5 vol. % to 25 vol. % (e.g., as a secondary solvent), or from greater than 25 vol. % to 90 vol. % (e.g., as a primary solvent), or within any range encompassed by any two of the foregoing values as endpoints, based on a total volume of the electrolyte. The amount of Fluorinated Esters 1-3 can be selected depending on a battery being formed and uses thereof. It is understood that the invention comprises, consists essentially of, or consists of the compositions identified in Table 90. It is understood that the invention comprises, consists essentially of, or consists of the compositions identified in Table 9E. It is understood that the invention comprises, consists essentially of, or consists of the compositions identified in Table 9F.

TABLE 9D

| | | Concentration of |
|---|---|---|
| Fluorinated Ester | Solvent | Fluorinated Ester 1 |
| Fluorinated Ester 1 | fluoroethylene carbonate (FEC) | 0.1 vol. %-5 vol. % |
| Fluorinated Ester 1 | fluoroethylene carbonate (FEC) | >5 vol. %-25 vol. % |
| Fluorinated Ester 1 | fluoroethylene carbonate (FEC) | >25 vol. %-95 vol. % |
| Fluorinated Ester 1 | propylene carbonate (PC) | 0.1 vol. %-5 vol. % |
| Fluorinated Ester 1 | propylene carbonate (PC) | >5 vol. %-25 vol. % |
| Fluorinated Ester 1 | propylene carbonate (PC) | >25 vol. %-95 vol. % |
| Fluorinated Ester 1 | dimethyl carbonate (DMC) | 0.1 vol. %-5 vol. % |
| Fluorinated Ester 1 | dimethyl carbonate (DMC) | >5 vol. %-25 vol. % |
| Fluorinated Ester 1 | dimethyl carbonate (DMC) | >25 vol. %-95 vol. % |
| Fluorinated Ester 1 | diethyl carbonate (DEC) | 0.1 vol. %-5 vol. % |
| Fluorinated Ester 1 | diethyl carbonate (DEC) | >5 vol. %-25 vol. % |
| Fluorinated Ester 1 | diethyl carbonate (DEC) | >25 vol. %-95 vol. % |
| Fluorinated Ester 1 | ethyl methyl carbonate (EMC) | 0.1 vol. %-5 vol. % |
| Fluorinated Ester 1 | ethyl methyl carbonate (EMC) | >5 vol. %-25 vol. % |
| Fluorinated Ester 1 | ethyl methyl carbonate (EMC) | >25 vol. %-95 vol. % |
| Fluorinated Ester 1 | vinyl carbonate (VC) | 0.1 vol. %-5 vol. % |
| Fluorinated Ester 1 | vinyl carbonate (VC) | >5 vol. %-25 vol. % |
| Fluorinated Ester 1 | vinyl carbonate (VC) | >25 vol. %-95 vol. % |
| Fluorinated Ester 1 | vinyl ethylene carbonate (VEC) | 0.1 vol. %-5 vol. % |
| Fluorinated Ester 1 | vinyl ethylene carbonate (VEC) | >5 vol. %-25 vol. % |
| Fluorinated Ester 1 | vinyl ethylene carbonate (VEC) | >25 vol. %-95 vol. % |
| Fluorinated Ester 1 | ethylene carbonate (EC) | 0.1 vol. %-5 vol. % |
| Fluorinated Ester 1 | ethylene carbonate (EC) | >5 vol. %-25 vol. % |
| Fluorinated Ester 1 | ethylene carbonate (EC) | >25 vol. %-95 vol. % |
| Fluorinated Ester 1 | difluoroethylene carbonate (DFEC) | 0.1 vol. %-5 vol. % |
| Fluorinated Ester 1 | difluoroethylene carbonate (DFEC) | >5 vol. %-25 vol. % |
| Fluorinated Ester 1 | difluoroethylene carbonate (DFEC) | >25 vol. %-95 vol. % |
| Fluorinated Ester 1 | 3,3,3-trifluoropropylene carbonate | 0.1 vol. %-5 vol. % |
| | (TFPC) | |
| Fluorinated Ester 1 | 3,3,3-trifluoropropylene carbonate | >5 vol. %-25 vol. % |
| | (TFPC) | |
| Fluorinated Ester 1 | 3,3,3-trifluoropropylene carbonate | >25 vol. %-95 vol. % |
| | (TFPC) | |
| Fluorinated Ester 1 | monofluoroethyl methyl carbonate | 0.1 vol. %-5 vol. % |
| | (F1EMC) | |
| Fluorinated Ester 1 | monofluoroethyl methyl carbonate | >5 vol. %-25 vol. % |
| | (F1EMC) | |
| Fluorinated Ester 1 | monofluoroethyl methyl carbonate | >25 vol. %-95 vol. % |
| | (F1EMC) | |
| Fluorinated Ester 1 | difluoroethyl methyl carbonate | 0.1 vol. %-5 vol. % |
| | (F2EMC) | |
| Fluorinated Ester 1 | difluoroethyl methyl carbonate | >5 vol. %-25 vol. % |
| | (F2EMC) | |
| Fluorinated Ester 1 | difluoroethyl methyl carbonate | >25 vol. %-95 vol. % |
| | (F2EMC) | |
| Fluorinated Ester 1 | trifluoroethyl methyl carbonate | 0.1 vol. %-5 vol. % |
| | (F3EMC) | |
| Fluorinated Ester 1 | trifluoroethyl methyl carbonate | >5 vol. %-25 vol. % |
| | (F3EMC) | |
| Fluorinated Ester 1 | trifluoroethyl methyl carbonate | >25 vol. %-95 vol. % |
| | (F3EMC) | |
| Fluorinated Ester 1 | bis(2,2,2-trifluoroethyl) carbonate | 0.1 vol. %-5 vol. % |
| | (TFEC) | |
| Fluorinated Ester 1 | bis(2,2,2-trifluoroethyl) carbonate | >5 vol. %-25 vol. % |
| | (TFEC) | |
| Fluorinated Ester 1 | bis(2,2,2-trifluoroethyl) carbonate | >25 vol. %-95 vol. % |
| | (TFEC) | |
| Fluorinated Ester 1 | 2,2-difluoroethyl ethyl carbonate | 0.1 vol. %-5 vol. % |
| | (DFDEC) | |
| Fluorinated Ester 1 | 2,2-difluoroethyl ethyl carbonate | >5 vol. %-25 vol. % |
| | (DFDEC) | |
| Fluorinated Ester 1 | 2,2-difluoroethyl ethyl carbonate | >25 vol. %-95 vol. % |
| | (DFDEC) | |
| Fluorinated Ester 1 | acetonitrile (AN) | 0.1 vol. %-5 vol. % |
| Fluorinated Ester 1 | acetonitrile (AN) | >5 vol. %-25 vol. % |
| Fluorinated Ester 1 | acetonitrile (AN) | >25 vol. %-95 vol. % |
| Fluorinated Ester 1 | fluoroacetonitrile | 0.1 vol. %-5 vol. % |
| Fluorinated Ester 1 | fluoroacetonitrile | >5 vol. %-25 vol. % |
| Fluorinated Ester 1 | fluoroacetonitrile | >25 vol. %-95 vol. % |
| Fluorinated Ester 1 | ethyl acetate (EA) | 0.1 vol. %-5 vol. % |
| Fluorinated Ester 1 | ethyl acetate (EA) | >5 vol. %-25 vol. % |
| Fluorinated Ester 1 | ethyl acetate (EA) | >25 vol. %-95 vol. % |
| Fluorinated Ester 1 | methyl acetate (MA) | 0.1 vol. %-5 vol. % |
| Fluorinated Ester 1 | methyl acetate (MA) | >5 vol. %-25 vol. % |
| Fluorinated Ester 1 | methyl acetate (MA) | >25 vol. %-95 vol. % |
| Fluorinated Ester 1 | methyl propanoate (MP) | 0.1 vol. %-5 vol. % |
| Fluorinated Ester 1 | methyl propanoate (MP) | >5 vol. %-25 vol. % |
| Fluorinated Ester 1 | methyl propanoate (MP) | >25 vol. %-95 vol. % |
| Fluorinated Ester 1 | methyl 3,3,3-trifluoropropanoate | 0.1 vol. %-5 vol. % |
| Fluorinated Ester 1 | methyl 3,3,3-trifluoropropanoate | >5 vol. %-25 vol. % |
| Fluorinated Ester 1 | methyl 3,3,3-trifluoropropanoate | >25 vol. %-95 vol. % |
| Fluorinated Ester 1 | ethyl 3,3,3-trifluoropropanoate | 0.1 vol. %-5 vol. % |
| Fluorinated Ester 1 | ethyl 3,3,3-trifluoropropanoate | >5 vol. %-25 vol. % |
| Fluorinated Ester 1 | ethyl 3,3,3-trifluoropropanoate | >25 vol. %-95 vol. % |
| Fluorinated Ester 1 | ethyl 2,2-difluoropropanoate | 0.1 vol. %-5 vol. % |
| Fluorinated Ester 1 | ethyl 2,2-difluoropropanoate | >5 vol. %-25 vol. % |
| Fluorinated Ester 1 | ethyl 2,2-difluoropropanoate | >25 vol. %-95 vol. % |
| Fluorinated Ester 1 | isopropyl trifluoroacetate | 0.1 vol. %-5 vol. % |
| Fluorinated Ester 1 | isopropyl trifluoroacetate | >5 vol. %-25 vol. % |
| Fluorinated Ester 1 | isopropyl trifluoroacetate | >25 vol. %-95 vol. % |
| Fluorinated Ester 1 | methyl 4,4,4-trifluorobutanoate | 0.1 vol. %-5 vol. % |
| Fluorinated Ester 1 | methyl 4,4,4-trifluorobutanoate | >5 vol. %-25 vol. % |
| Fluorinated Ester 1 | methyl 4,4,4-trifluorobutanoate | >25 vol. %-95 vol. % |
| Fluorinated Ester 1 | methyl difluoroacetate | 0.1 vol. %-5 vol. % |
| Fluorinated Ester 1 | methyl difluoroacetate | >5 vol. %-25 vol. % |
| Fluorinated Ester 1 | methyl difluoroacetate | >25 vol. %-95 vol. % |
| Fluorinated Ester 1 | 2,2,2-trifluoroethyl butanoate | 0.1 vol. %-5 vol. % |
| Fluorinated Ester 1 | 2,2,2-trifluoroethyl butanoate | >5 vol. %-25 vol. % |
| Fluorinated Ester 1 | 2,2,2-trifluoroethyl butanoate | >25 vol. %-95 vol. % |
| Fluorinated Ester 1 | methyl 2,2-difluoropropanoate | 0.1 vol. %-5 vol. % |
| Fluorinated Ester 1 | methyl 2,2-difluoropropanoate | >5 vol. %-25 vol. % |
| Fluorinated Ester 1 | methyl 2,2-difluoropropanoate | >25 vol. %-95 vol. % |
| Fluorinated Ester 1 | ethyl trifluoroacetate | 0.1 vol. %-5 vol. % |
| Fluorinated Ester 1 | ethyl trifluoroacetate | >5 vol. %-25 vol. % |
| Fluorinated Ester 1 | ethyl trifluoroacetate | >25 vol. %-95 vol. % |
| Fluorinated Ester 1 | 2,2-difluoroethyl trifluoroacetate | 0.1 vol. %-5 vol. % |
| Fluorinated Ester 1 | 2,2-difluoroethyl trifluoroacetate | >5 vol. %-25 vol. % |
| Fluorinated Ester 1 | 2,2-difluoroethyl trifluoroacetate | >25 vol. %-95 vol. % |
| Fluorinated Ester 1 | 2,2,2-trifluoroethyl acetate | 0.1 vol. %-5 vol. % |
| Fluorinated Ester 1 | 2,2,2-trifluoroethyl acetate | >5 vol. %-25 vol. % |
| Fluorinated Ester 1 | 2,2,2-trifluoroethyl acetate | >25 vol. %-95 vol. % |
| Fluorinated Ester 1 | ethyl 2,2-difluoroacetate | 0.1 vol. %-5 vol. % |
| Fluorinated Ester 1 | ethyl 2,2-difluoroacetate | >5 vol. %-25 vol. % |
| Fluorinated Ester 1 | ethyl 2,2-difluoroacetate | >25 vol. %-95 vol. % |
| Fluorinated Ester 1 | 2,2-difluoroethyl acetate | 0.1 vol. %-5 vol. % |
| Fluorinated Ester 1 | 2,2-difluoroethyl acetate | >5 vol. %-25 vol. % |
| Fluorinated Ester 1 | 2,2-difluoroethyl acetate | >25 vol. %-95 vol. % |
| Fluorinated Ester 1 | 2,2-difluoroethyl 3,3- | 0.1 vol. %-5 vol. % |
| | difluoropropanoate | |
| Fluorinated Ester 1 | 2,2-difluoroethyl 3,3- | >5 vol. %-25 vol. % |
| | difluoropropanoate | |
| Fluorinated Ester 1 | 2,2-difluoroethyl 3,3- | >25 vol. %-95 vol. % |
| | difluoropropanoate | |
| Fluorinated Ester 1 | 2,2-difluoroethyl 2,2-difluoroacetate | 0.1 vol. %-5 vol. % |
| Fluorinated Ester 1 | 2,2-difluoroethyl 2,2-difluoroacetate | >5 vol. %-25 vol. % |
| Fluorinated Ester 1 | 2,2-difluoroethyl 2,2-difluoroacetate | >25 vol. %-95 vol. % |
| Fluorinated Ester 1 | succinonitrile (SN) | 0.1 vol. %-5 vol. % |
| Fluorinated Ester 1 | succinonitrile (SN) | >5 vol. %-25 vol. % |
| Fluorinated Ester 1 | succinonitrile (SN) | >25 vol. %-95 vol. % |
| Fluorinated Ester 1 | propionitrile (PN) | 0.1 vol. %-5 vol. % |
| Fluorinated Ester 1 | propionitrile (PN) | >5 vol. %-25 vol. % |
| Fluorinated Ester 1 | propionitrile (PN) | >25 vol. %-95 vol. % |
| Fluorinated Ester 1 | butyronitrile (BN) | 0.1 vol. %-5 vol. % |
| Fluorinated Ester 1 | butyronitrile (BN) | >5 vol. %-25 vol. % |
| Fluorinated Ester 1 | butyronitrile (BN) | >25 vol. %-95 vol. % |
| Fluorinated Ester 1 | adiponitrile (ADN) | 0.1 vol. %-5 vol. % |
| Fluorinated Ester 1 | adiponitrile (ADN) | >5 vol. %-25 vol. % |
| Fluorinated Ester 1 | adiponitrile (ADN) | >25 vol. %-95 vol. % |
| Fluorinated Ester 1 | 1,3,6-hexanetricarbonitrile (HTCN) | 0.1 vol. %-5 vol. % |
| Fluorinated Ester 1 | 1,3,6-hexanetricarbonitrile (HTCN) | >5 vol. %-25 vol. % |
| Fluorinated Ester 1 | 1,3,6-hexanetricarbonitrile (HTCN) | >25 vol. %-95 vol. % |
| Fluorinated Ester 1 | trimethyl borate (TMB) | 0.1 vol. %-5 vol. % |
| Fluorinated Ester 1 | trimethyl borate (TMB) | >5 vol. %-25 vol. % |
| Fluorinated Ester 1 | trimethyl borate (TMB) | >25 vol. %-95 vol. % |
| Fluorinated Ester 1 | triphenyl borate (TPB) | 0.1 vol. %-5 vol. % |
| Fluorinated Ester 1 | triphenyl borate (TPB) | >5 vol. %-25 vol. % |
| Fluorinated Ester 1 | triphenyl borate (TPB) | >25 vol. %-95 vol. % |
| Fluorinated Ester 1 | triethyl borate (TEB) | 0.1 vol. %-5 vol. % |
| Fluorinated Ester 1 | triethyl borate (TEB) | >5 vol. %-25 vol. % |
| Fluorinated Ester 1 | triethyl borate (TEB) | >25 vol. %-95 vol. % |
| Fluorinated Ester 1 | tris(pentafluorophenyl)borane | 0.1 vol. %-5 vol. % |
| | (TPFPB) | |
| Fluorinated Ester 1 | tris(pentafluorophenyl)borane | >5 vol. %-25 vol. % |
| | (TPFPB) | |
| Fluorinated Ester 1 | tris(pentafluorophenyl)borane | >25 vol. %-95 vol. % |
| | (TPFPB) | |
| Fluorinated Ester 1 | tris(trimethylsilyl)phosphate (TTSB) | 0.1 vol. %-5 vol. % |
| Fluorinated Ester 1 | tris(trimethylsilyl)phosphate (TTSB) | >5 vol. %-25 vol. % |
| Fluorinated Ester 1 | tris(trimethylsilyl)phosphate (TTSB) | >25 vol. %-95 vol. % |
| Fluorinated Ester 1 | tris(2,2,2-trifluoroethyl) borate | 0.1 vol. %-5 vol. % |
| | (TTFEB) | |
| Fluorinated Ester 1 | tris(2,2,2-trifluoroethyl) borate | >5 vol. %-25 vol. % |
| | (TTFEB) | |
| Fluorinated Ester 1 | tris(2,2,2-trifluoroethyl) borate | >25 vol. %-95 vol. % |
| | (TTFEB) | |
| Fluorinated Ester 1 | trimethyl phosphate (TMP) | 0.1 vol. %-5 vol. % |
| Fluorinated Ester 1 | trimethyl phosphate (TMP) | >5 vol. %-25 vol. % |
| Fluorinated Ester 1 | trimethyl phosphate (TMP) | >25 vol. %-95 vol. % |
| Fluorinated Ester 1 | triethyl phosphate (TEP) | 0.1 vol. %-5 vol. % |
| Fluorinated Ester 1 | triethyl phosphate (TEP) | >5 vol. %-25 vol. % |
| Fluorinated Ester 1 | triethyl phosphate (TEP) | >25 vol. %-95 vol. % |
| Fluorinated Ester 1 | tris(trimethylsilyl)phosphate (TTSP) | 0.1 vol. %-5 vol. % |
| Fluorinated Ester 1 | tris(trimethylsilyl)phosphate (TTSP) | >5 vol. %-25 vol. % |
| Fluorinated Ester 1 | tris(trimethylsilyl)phosphate (TTSP) | >25 vol. %-95 vol. % |
| Fluorinated Ester 1 | tris(trimethylsilyl)phosphite (TTSPi) | 0.1 vol. %-5 vol. % |
| Fluorinated Ester 1 | tris(trimethylsilyl)phosphite (TTSPi) | >5 vol. %-25 vol. % |
| Fluorinated Ester 1 | tris(trimethylsilyl)phosphite (TTSPi) | >25 vol. %-95 vol. % |
| Fluorinated Ester 1 | tris(2,2,2-trifluoroethyl) phosphate | 0.1 vol. %-5 vol. % |
| | (TFEPa) | |
| Fluorinated Ester 1 | tris(2,2,2-trifluoroethyl) phosphate | >5 vol. %-25 vol. % |
| | (TFEPa) | |
| Fluorinated Ester 1 | tris(2,2,2-trifluoroethyl) phosphate | >25 vol. %-95 vol. % |
| | (TFEPa) | |
| Fluorinated Ester 1 | tris(2,2,2-trifluoroethyl) phosphite | 0.1 vol. %-5 vol. % |
| | (TFEPi) | |
| Fluorinated Ester 1 | tris(2,2,2-trifluoroethyl) phosphite | >5 vol. %-25 vol. % |
| | (TFEPi) | |
| Fluorinated Ester 1 | tris(2,2,2-trifluoroethyl) phosphite | >25 vol. %-95 vol. % |
| | (TFEPi) | |
| Fluorinated Ester 1 | (pentafluorophenyl)diphenyl | 0.1 vol. %-5 vol. % |
| | phosphine (PFPDPP) | |
| Fluorinated Ester 1 | (pentafluorophenyl)diphenyl | >5 vol. %-25 vol. % |
| | phosphine (PFPDPP) | |
| Fluorinated Ester 1 | (pentafluorophenyl)diphenyl | >25 vol. %-95 vol. % |
| | phosphine (PFPDPP) | |
| Fluorinated Ester 1 | tris(pentafluorophenyl) phosphine | 0.1 vol. %-5 vol. % |
| | (TPFPP) | |
| Fluorinated Ester 1 | tris(pentafluorophenyl) phosphine | >5 vol. %-25 vol. % |
| | (TPFPP) | |
| Fluorinated Ester 1 | tris(pentafluorophenyl) phosphine | >25 vol. %-95 vol. % |
| | (TPFPP) | |
| Fluorinated Ester 1 | 1,3,2-dioxathiolane-2,2-dioxide (DTD) | 0.1 vol. %-5 vol. % |
| Fluorinated Ester 1 | 1,3,2-dioxathiolane-2,2-dioxide (DTD) | >5 vol. %-25 vol. % |
| Fluorinated Ester 1 | 1,3,2-dioxathiolane-2,2-dioxide (DTD) | >25 vol. %-95 vol. % |
| Fluorinated Ester 1 | 1,3-propanesultone (PS) | 0.1 vol. %-5 vol. % |
| Fluorinated Ester 1 | 1,3-propanesultone (PS) | >5 vol. %-25 vol. % |
| Fluorinated Ester 1 | 1,3-propanesultone (PS) | >25 vol. %-95 vol. % |
| Fluorinated Ester 1 | prop-1-ene-1,3-sultone (PES) | 0.1 vol. %-5 vol. % |
| Fluorinated Ester 1 | prop-1-ene-1,3-sultone (PES) | >5 vol. %-25 vol. % |
| Fluorinated Ester 1 | prop-1-ene-1,3-sultone (PES) | >25 vol. %-95 vol. % |
| Fluorinated Ester 1 | propanediol cyclic sulfate (PCS) | 0.1 vol. %-5 vol. % |
| Fluorinated Ester 1 | propanediol cyclic sulfate (PCS) | >5 vol. %-25 vol. % |
| Fluorinated Ester 1 | propanediol cyclic sulfate (PCS) | >25 vol. %-95 vol. % |
| Fluorinated Ester 1 | 1,3,2-dioxathiolane 2-oxide | 0.1 vol. %-5 vol. % |
| Fluorinated Ester 1 | 1,3,2-dioxathiolane 2-oxide | >5 vol. %-25 vol. % |
| Fluorinated Ester 1 | 1,3,2-dioxathiolane 2-oxide | >25 vol. %-95 vol. % |
| Fluorinated Ester 1 | bis(2,2,2-trifluoroethyl) ether (BTFE) | 0.1 vol. %-5 vol. % |
| Fluorinated Ester 1 | bis(2,2,2-trifluoroethyl) ether (BTFE) | >5 vol. %-25 vol. % |
| Fluorinated Ester 1 | bis(2,2,2-trifluoroethyl) ether (BTFE) | >25 vol. %-95 vol. % |
| Fluorinated Ester 1 | 1,1,2,2-tetrafluoroethyl-2,2,3,3- | 0.1 vol. %-5 vol. % |
| | tetrafluoropropyl ether (TTE) | |
| Fluorinated Ester 1 | 1,1,2,2-tetrafluoroethyl-2,2,3,3- | >5 vol. %-25 vol. % |
| | tetrafluoropropyl ether (TTE) | |
| Fluorinated Ester 1 | 1,1,2,2-tetrafluoroethyl-2,2,3,3- | >25 vol. %-95 vol. % |
| | tetrafluoropropyl ether (TTE) | |
| Fluorinated Ester 1 | 1H,1H,5H-octafluoropentyl-1,1,2,2- | 0.1 vol. %-5 vol. % |
| | tetrafluoroethylether (OTE) | |
| Fluorinated Ester 1 | 1H,1H,5H-octafluoropentyl-1,1,2,2- | >5 vol. %-25 vol. % |
| | tetrafluoroethylether (OTE) | |
| Fluorinated Ester 1 | 1H,1H,5H-octafluoropentyl-1,1,2,2- | >25 vol. %-95 vol. % |
| | tetrafluoroethylether (OTE) | |
| Fluorinated Ester 1 | 2,2,2-trifluoroethyl 1,1,2,2- | 0.1 vol. %-5 vol. % |
| | tetrafluoroethyl ether (HFE) | |
| Fluorinated Ester 1 | 2,2,2-trifluoroethyl 1,1,2,2- | >5 vol. %-25 vol. % |
| | tetrafluoroethyl ether (HFE) | |
| Fluorinated Ester 1 | 2,2,2-trifluoroethyl 1,1,2,2- | >25 vol. %-95 vol. % |
| | tetrafluoroethyl ether (HFE) | |
| Fluorinated Ester 1 | tetrahydrofuran (THF) | 0.1 vol. %-5 vol. % |
| Fluorinated Ester 1 | tetrahydrofuran (THF) | >5 vol. %-25 vol. % |
| Fluorinated Ester 1 | tetrahydrofuran (THF) | >25 vol. %-95 vol. % |
| Fluorinated Ester 1 | 1,3-dioxolane (DOL) | 0.1 vol. %-5 vol. % |
| Fluorinated Ester 1 | 1,3-dioxolane (DOL) | >5 vol. %-25 vol. % |
| Fluorinated Ester 1 | 1,3-dioxolane (DOL) | >25 vol. %-95 vol. % |
| Fluorinated Ester 1 | 1,2-dimethoxyethane (DME) | 0.1 vol. %-5 vol. % |
| Fluorinated Ester 1 | 1,2-dimethoxyethane (DME) | >5 vol. %-25 vol. % |
| Fluorinated Ester 1 | 1,2-dimethoxyethane (DME) | >25 vol. %-95 vol. % |
| Fluorinated Ester 1 | diethylene glycol dimethyl ether | 0.1 vol. %-5 vol. % |
| | (DEGDME) | |
| Fluorinated Ester 1 | diethylene glycol dimethyl ether | >5 vol. %-25 vol. % |
| | (DEGDME) | |
| Fluorinated Ester 1 | diethylene glycol dimethyl ether | >25 vol. %-95 vol. % |
| | (DEGDME) | |
| Fluorinated Ester 1 | tetraethylene glycol dimethyl ether | 0.1 vol. %-5 vol. % |
| | (TEGDME) | |
| Fluorinated Ester 1 | tetraethylene glycol dimethyl ether | >5 vol. %-25 vol. % |
| | (TEGDME) | |
| Fluorinated Ester 1 | tetraethylene glycol dimethyl ether | >25 vol. %-95 vol. % |
| | (TEGDME) | |
| Fluorinated Ester 1 | succinic anhydride (SA) | 0.1 vol. %-5 vol. % |
| Fluorinated Ester 1 | succinic anhydride (SA) | >5 vol. %-25 vol. % |
| Fluorinated Ester 1 | succinic anhydride (SA) | >25 vol. %-95 vol. % |
| Fluorinated Ester 1 | butyric anhydride (BA) | 0.1 vol. %-5 vol. % |
| Fluorinated Ester 1 | butyric anhydride (BA) | >5 vol. %-25 vol. % |
| Fluorinated Ester 1 | butyric anhydride (BA) | >25 vol. %-95 vol. % |
| Fluorinated Ester 1 | 1,4-dioxane (DOX) | 0.1 vol. %-5 vol. % |
| Fluorinated Ester 1 | 1,4-dioxane (DOX) | >5 vol. %-25 vol. % |
| Fluorinated Ester 1 | 1,4-dioxane (DOX) | >25 vol. %-95 vol. % |
| Fluorinated Ester 1 | tetravinyl silane (TVSI) | 0.1 vol. %-5 vol. % |
| Fluorinated Ester 1 | tetravinyl silane (TVSI) | >5 vol. %-25 vol. % |
| Fluorinated Ester 1 | tetravinyl silane (TVSI) | >25 vol. %-95 vol. % |
| Fluorinated Ester 1 | 1,4-butane sultone (BS) | 0.1 vol. %-5 vol. % |
| Fluorinated Ester 1 | 1,4-butane sultone (BS) | >5 vol. %-25 vol. % |
| Fluorinated Ester 1 | 1,4-butane sultone (BS) | >25 vol. %-95 vol. % |
| Fluorinated Ester 1 | dimethyl sulfoxide (DMSO) | 0.1 vol. %-5 vol. % |
| Fluorinated Ester 1 | dimethyl sulfoxide (DMSO) | >5 vol. %-25 vol. % |
| Fluorinated Ester 1 | dimethyl sulfoxide (DMSO) | >25 vol. %-95 vol. % |
| Fluorinated Ester 1 | methylene methanedisulfonate | 0.1 vol. %-5 vol. % |
| | (MMDS) | |
| Fluorinated Ester 1 | methylene methanedisulfonate | >5 vol. %-25 vol. % |
| | (MMDS) | |
| Fluorinated Ester 1 | methylene methanedisulfonate | >25 vol. %-95 vol. % |
| | (MMDS) | |
| Fluorinated Ester 1 | N,N-dimethylformamide (DMF) | 0.1 vol. %-5 vol. % |
| Fluorinated Ester 1 | N,N-dimethylformamide (DMF) | >5 vol. %-25 vol. % |
| Fluorinated Ester 1 | N,N-dimethylformamide (DMF) | >25 vol. %-95 vol. % |
| Fluorinated Ester 1 | gamma-butyrolactone (BL) | 0.1 vol. %-5 vol. % |
| Fluorinated Ester 1 | gamma-butyrolactone (BL) | >5 vol. %-25 vol. % |
| Fluorinated Ester 1 | gamma-butyrolactone (BL) | >25 vol. %-95 vol. % |

TABLE 9E

| | | Concentration of |
|---|---|---|
| Fluorinated Ester | Solvent | Fluorinated Ester 2 |
| Fluorinated Ester 2 | fluoroethylene carbonate (FEC) | 0.1 vol. %-5 vol. % |
| Fluorinated Ester 2 | fluoroethylene carbonate (FEC) | >5 vol. %-25 vol. % |
| Fluorinated Ester 2 | fluoroethylene carbonate (FEC) | >25 vol. %-95 vol. % |
| Fluorinated Ester 2 | propylene carbonate (PC) | 0.1 vol. %-5 vol. % |
| Fluorinated Ester 2 | propylene carbonate (PC) | >5 vol. %-25 vol. % |
| Fluorinated Ester 2 | propylene carbonate (PC) | >25 vol. %-95 vol. % |
| Fluorinated Ester 2 | dimethyl carbonate (DMC) | 0.1 vol. %-5 vol. % |
| Fluorinated Ester 2 | dimethyl carbonate (DMC) | >5 vol. %-25 vol. % |
| Fluorinated Ester 2 | dimethyl carbonate (DMC) | >25 vol. %-95 vol. % |
| Fluorinated Ester 2 | diethyl carbonate (DEC) | 0.1 vol. %-5 vol. % |
| Fluorinated Ester 2 | diethyl carbonate (DEC) | >5 vol. %-25 vol. % |
| Fluorinated Ester 2 | diethyl carbonate (DEC) | >25 vol. %-95 vol. % |
| Fluorinated Ester 2 | ethyl methyl carbonate (EMC) | 0.1 vol. %-5 vol. % |
| Fluorinated Ester 2 | ethyl methyl carbonate (EMC) | >5 vol. %-25 vol. % |
| Fluorinated Ester 2 | ethyl methyl carbonate (EMC) | >25 vol. %-95 vol. % |
| Fluorinated Ester 2 | vinyl carbonate (VC) | 0.1 vol. %-5 vol. % |
| Fluorinated Ester 2 | vinyl carbonate (VC) | >5 vol. %-25 vol. % |
| Fluorinated Ester 2 | vinyl carbonate (VC) | >25 vol. %-95 vol. % |
| Fluorinated Ester 2 | vinyl ethylene carbonate (VEC) | 0.1 vol. %-5 vol. % |
| Fluorinated Ester 2 | vinyl ethylene carbonate (VEC) | >5 vol. %-25 vol. % |
| Fluorinated Ester 2 | vinyl ethylene carbonate (VEC) | >25 vol. %-95 vol. % |
| Fluorinated Ester 2 | ethylene carbonate (EC) | 0.1 vol. %-5 vol. % |
| Fluorinated Ester 2 | ethylene carbonate (EC) | >5 vol. %-25 vol. % |
| Fluorinated Ester 2 | ethylene carbonate (EC) | >25 vol. %-95 vol. % |
| Fluorinated Ester 2 | difluoroethylene carbonate (DFEC) | 0.1 vol. %-5 vol. % |
| Fluorinated Ester 2 | difluoroethylene carbonate (DFEC) | >5 vol. %-25 vol. % |
| Fluorinated Ester 2 | difluoroethylene carbonate (DFEC) | >25 vol. %-95 vol. % |
| Fluorinated Ester 2 | 3,3,3-trifluoropropylene carbonate | 0.1 vol. %-5 vol. % |
| | (TFPC) | |
| Fluorinated Ester 2 | 3,3,3-trifluoropropylene carbonate | >5 vol. %-25 vol. % |
| | (TFPC) | |
| Fluorinated Ester 2 | 3,3,3-trifluoropropylene carbonate | >25 vol. %-95 vol. % |
| | (TFPC) | |
| Fluorinated Ester 2 | monofluoroethyl methyl carbonate | 0.1 vol. %-5 vol. % |
| | (F1EMC) | |
| Fluorinated Ester 2 | monofluoroethyl methyl carbonate | >5 vol. %-25 vol. % |
| | (F1EMC) | |
| Fluorinated Ester 2 | monofluoroethyl methyl carbonate | >25 vol. %-95 vol. % |
| | (F1EMC) | |
| Fluorinated Ester 2 | difluoroethyl methyl carbonate | 0.1 vol. %-5 vol. % |
| | (F2EMC) | |
| Fluorinated Ester 2 | difluoroethyl methyl carbonate | >5 vol. %-25 vol. % |
| | (F2EMC) | |
| Fluorinated Ester 2 | difluoroethyl methyl carbonate | >25 vol. %-95 vol. % |
| | (F2EMC) | |
| Fluorinated Ester 2 | trifluoroethyl methyl carbonate | 0.1 vol. %-5 vol. % |
| | (F3EMC) | |
| Fluorinated Ester 2 | trifluoroethyl methyl carbonate | >5 vol. %-25 vol. % |
| | (F3EMC) | |
| Fluorinated Ester 2 | trifluoroethyl methyl carbonate | >25 vol. %-95 vol. % |
| | (F3EMC) | |
| Fluorinated Ester 2 | bis(2,2,2-trifluoroethyl) carbonate | 0.1 vol. %-5 vol. % |
| | (TFEC) | |
| Fluorinated Ester 2 | bis(2,2,2-trifluoroethyl) carbonate | >5 vol. %-25 vol. % |
| | (TFEC) | |
| Fluorinated Ester 2 | bis(2,2,2-trifluoroethyl) carbonate | >25 vol. %-95 vol. % |
| | (TFEC) | |
| Fluorinated Ester 2 | 2,2-difluoroethyl ethyl carbonate | 0.1 vol. %-5 vol. % |
| | (DFDEC) | |
| Fluorinated Ester 2 | 2,2-difluoroethyl ethyl carbonate | >5 vol. %-25 vol. % |
| | (DFDEC) | |
| Fluorinated Ester 2 | 2,2-difluoroethyl ethyl carbonate | >25 vol. %-95 vol. % |
| | (DFDEC) | |
| Fluorinated Ester 2 | acetonitrile (AN) | 0.1 vol. %-5 vol. % |
| Fluorinated Ester 2 | acetonitrile (AN) | >5 vol. %-25 vol. % |
| Fluorinated Ester 2 | acetonitrile (AN) | >25 vol. %-95 vol. % |
| Fluorinated Ester 2 | fluoroacetonitrile | 0.1 vol. %-5 vol. % |
| Fluorinated Ester 2 | fluoroacetonitrile | >5 vol. %-25 vol. % |
| Fluorinated Ester 2 | fluoroacetonitrile | >25 vol. %-95 vol. % |
| Fluorinated Ester 2 | ethyl acetate (EA) | 0.1 vol. %-5 vol. % |
| Fluorinated Ester 2 | ethyl acetate (EA) | >5 vol. %-25 vol. % |
| Fluorinated Ester 2 | ethyl acetate (EA) | >25 vol. %-95 vol. % |
| Fluorinated Ester 2 | methyl acetate (MA) | 0.1 vol. %-5 vol. % |
| Fluorinated Ester 2 | methyl acetate (MA) | >5 vol. %-25 vol. % |
| Fluorinated Ester 2 | methyl acetate (MA) | >25 vol. %-95 vol. % |
| Fluorinated Ester 2 | methyl propanoate (MP) | 0.1 vol. %-5 vol. % |
| Fluorinated Ester 2 | methyl propanoate (MP) | >5 vol. %-25 vol. % |
| Fluorinated Ester 2 | methyl propanoate (MP) | >25 vol. %-95 vol. % |
| Fluorinated Ester 2 | methyl 3,3,3-trifluoropropanoate | 0.1 vol. %-5 vol. % |
| Fluorinated Ester 2 | methyl 3,3,3-trifluoropropanoate | >5 vol. %-25 vol. % |
| Fluorinated Ester 2 | methyl 3,3,3-trifluoropropanoate | >25 vol. %-95 vol. % |
| Fluorinated Ester 2 | ethyl 3,3,3-trifluoropropanoate | 0.1 vol. %-5 vol. % |
| Fluorinated Ester 2 | ethyl 3,3,3-trifluoropropanoate | >5 vol. %-25 vol. % |
| Fluorinated Ester 2 | ethyl 3,3,3-trifluoropropanoate | >25 vol. %-95 vol. % |
| Fluorinated Ester 2 | ethyl 2,2-difluoropropanoate | 0.1 vol. %-5 vol. % |
| Fluorinated Ester 2 | ethyl 2,2-difluoropropanoate | >5 vol. %-25 vol. % |
| Fluorinated Ester 2 | ethyl 2,2-difluoropropanoate | >25 vol. %-95 vol. % |
| Fluorinated Ester 2 | isopropyl trifluoroacetate | 0.1 vol. %-5 vol. % |
| Fluorinated Ester 2 | isopropyl trifluoroacetate | >5 vol. %-25 vol. % |
| Fluorinated Ester 2 | isopropyl trifluoroacetate | >25 vol. %-95 vol. % |
| Fluorinated Ester 2 | methyl 4,4,4-trifluorobutanoate | 0.1 vol. %-5 vol. % |
| Fluorinated Ester 2 | methyl 4,4,4-trifluorobutanoate | >5 vol. %-25 vol. % |
| Fluorinated Ester 2 | methyl 4,4,4-trifluorobutanoate | >25 vol. %-95 vol. % |
| Fluorinated Ester 2 | methyl difluoroacetate | 0.1 vol. %-5 vol. % |
| Fluorinated Ester 2 | methyl difluoroacetate | >5 vol. %-25 vol. % |
| Fluorinated Ester 2 | methyl difluoroacetate | >25 vol. %-95 vol. % |
| Fluorinated Ester 2 | 2,2,2-trifluoroethyl butanoate | 0.1 vol. %-5 vol. % |
| Fluorinated Ester 2 | 2,2,2-trifluoroethyl butanoate | >5 vol. %-25 vol. % |
| Fluorinated Ester 2 | 2,2,2-trifluoroethyl butanoate | >25 vol. %-95 vol. % |
| Fluorinated Ester 2 | methyl 2,2-difluoropropanoate | 0.1 vol. %-5 vol. % |
| Fluorinated Ester 2 | methyl 2,2-difluoropropanoate | >5 vol. %-25 vol. % |
| Fluorinated Ester 2 | methyl 2,2-difluoropropanoate | >25 vol. %-95 vol. % |
| Fluorinated Ester 2 | ethyl trifluoroacetate | 0.1 vol. %-5 vol. % |
| Fluorinated Ester 2 | ethyl trifluoroacetate | >5 vol. %-25 vol. % |
| Fluorinated Ester 2 | ethyl trifluoroacetate | >25 vol. %-95 vol. % |
| Fluorinated Ester 2 | 2,2-difluoroethyl trifluoroacetate | 0.1 vol. %-5 vol. % |
| Fluorinated Ester 2 | 2,2-difluoroethyl trifluoroacetate | >5 vol. %-25 vol. % |
| Fluorinated Ester 2 | 2,2-difluoroethyl trifluoroacetate | >25 vol. %-95 vol. % |
| Fluorinated Ester 2 | 2,2,2-trifluoroethyl acetate | 0.1 vol. %-5 vol. % |
| Fluorinated Ester 2 | 2,2,2-trifluoroethyl acetate | >5 vol. %-25 vol. % |
| Fluorinated Ester 2 | 2,2,2-trifluoroethyl acetate | >25 vol. %-95 vol. % |
| Fluorinated Ester 2 | ethyl 2,2-difluoroacetate | 0.1 vol. %-5 vol. % |
| Fluorinated Ester 2 | ethyl 2,2-difluoroacetate | >5 vol. %-25 vol. % |
| Fluorinated Ester 2 | ethyl 2,2-difluoroacetate | >25 vol. %-95 vol. % |
| Fluorinated Ester 2 | 2,2-difluoroethyl acetate | 0.1 vol. %-5 vol. % |
| Fluorinated Ester 2 | 2,2-difluoroethyl acetate | >5 vol. %-25 vol. % |
| Fluorinated Ester 2 | 2,2-difluoroethyl acetate | >25 vol. %-95 vol. % |
| Fluorinated Ester 2 | 2,2-difluoroethyl 3,3- | 0.1 vol. %-5 vol. % |
| | difluoropropanoate | |
| Fluorinated Ester 2 | 2,2-difluoroethyl 3,3- | >5 vol. %-25 vol. % |
| | difluoropropanoate | |
| Fluorinated Ester 2 | 2,2-difluoroethyl 3,3- | >25 vol. %-95 vol. % |
| | difluoropropanoate | |
| Fluorinated Ester 2 | 2,2-difluoroethyl 2,2-difluoroacetate | 0.1 vol. %-5 vol. % |
| Fluorinated Ester 2 | 2,2-difluoroethyl 2,2-difluoroacetate | >5 vol. %-25 vol. % |
| Fluorinated Ester 2 | 2,2-difluoroethyl 2,2-difluoroacetate | >25 vol. %-95 vol. % |
| Fluorinated Ester 2 | succinonitrile (SN) | 0.1 vol. %-5 vol. % |
| Fluorinated Ester 2 | succinonitrile (SN) | >5 vol. %-25 vol. % |
| Fluorinated Ester 2 | succinonitrile (SN) | >25 vol. %-95 vol. % |
| Fluorinated Ester 2 | propionitrile (PN) | 0.1 vol. %-5 vol. % |
| Fluorinated Ester 2 | propionitrile (PN) | >5 vol. %-25 vol. % |
| Fluorinated Ester 2 | propionitrile (PN) | >25 vol. %-95 vol. % |
| Fluorinated Ester 2 | butyronitrile (BN) | 0.1 vol. %-5 vol. % |
| Fluorinated Ester 2 | butyronitrile (BN) | >5 vol. %-25 vol. % |
| Fluorinated Ester 2 | butyronitrile (BN) | >25 vol. %-95 vol. % |
| Fluorinated Ester 2 | adiponitrile (ADN) | 0.1 vol. %-5 vol. % |
| Fluorinated Ester 2 | adiponitrile (ADN) | >5 vol. %-25 vol. % |
| Fluorinated Ester 2 | adiponitrile (ADN) | >25 vol. %-95 vol. % |
| Fluorinated Ester 2 | 1,3,6-hexanetricarbonitrile (HTCN) | 0.1 vol. %-5 vol. % |
| Fluorinated Ester 2 | 1,3,6-hexanetricarbonitrile (HTCN) | >5 vol. %-25 vol. % |
| Fluorinated Ester 2 | 1,3,6-hexanetricarbonitrile (HTCN) | >25 vol. %-95 vol. % |
| Fluorinated Ester 2 | trimethyl borate (TMB) | 0.1 vol. %-5 vol. % |
| Fluorinated Ester 2 | trimethyl borate (TMB) | >5 vol. %-25 vol. % |
| Fluorinated Ester 2 | trimethyl borate (TMB) | >25 vol. %-95 vol. % |
| Fluorinated Ester 2 | triphenyl borate (TPB) | 0.1 vol. %-5 vol. % |
| Fluorinated Ester 2 | triphenyl borate (TPB) | >5 vol. %-25 vol. % |
| Fluorinated Ester 2 | triphenyl borate (TPB) | >25 vol. %-95 vol. % |
| Fluorinated Ester 2 | triethyl borate (TEB) | 0.1 vol. %-5 vol. % |
| Fluorinated Ester 2 | triethyl borate (TEB) | >5 vol. %-25 vol. % |
| Fluorinated Ester 2 | triethyl borate (TEB) | >25 vol. %-95 vol. % |
| Fluorinated Ester 2 | tris(pentafluorophenyl)borane (TPFPB) | 0.1 vol. %-5 vol. % |
| Fluorinated Ester 2 | tris(pentafluorophenyl)borane (TPFPB) | >5 vol. %-25 vol. % |
| Fluorinated Ester 2 | tris(pentafluorophenyl)borane (TPFPB) | >25 vol. %-95 vol. % |
| Fluorinated Ester 2 | tris(trimethylsilyl)phosphate (TTSB) | 0.1 vol. %-5 vol. % |
| Fluorinated Ester 2 | tris(trimethylsilyl)phosphate (TTSB) | >5 vol. %-25 vol. % |
| Fluorinated Ester 2 | tris(trimethylsilyl)phosphate (TTSB) | >25 vol. %-95 vol. % |
| Fluorinated Ester 2 | tris(2,2,2-trifluoroethyl) borate (TTFEB) | 0.1 vol. %-5 vol. % |
| Fluorinated Ester 2 | tris(2,2,2-trifluoroethyl) borate (TTFEB) | >5 vol. %-25 vol. % |
| Fluorinated Ester 2 | tris(2,2,2-trifluoroethyl) borate (TTFEB) | >25 vol. %-95 vol. % |
| Fluorinated Ester 2 | trimethyl phosphate (TMP) | 0.1 vol. %-5 vol. % |
| Fluorinated Ester 2 | trimethyl phosphate (TMP) | >5 vol. %-25 vol. % |
| Fluorinated Ester 2 | trimethyl phosphate (TMP) | >25 vol. %-95 vol. % |
| Fluorinated Ester 2 | triethyl phosphate (TEP) | 0.1 vol. %-5 vol. % |
| Fluorinated Ester 2 | triethyl phosphate (TEP) | >5 vol. %-25 vol. % |
| Fluorinated Ester 2 | triethyl phosphate (TEP) | >25 vol. %-95 vol. % |
| Fluorinated Ester 2 | tris(trimethylsilyl)phosphate (TTSP) | 0.1 vol. %-5 vol. % |
| Fluorinated Ester 2 | tris(trimethylsilyl)phosphate (TTSP) | >5 vol. %-25 vol. % |
| Fluorinated Ester 2 | tris(trimethylsilyl)phosphate (TTSP) | >25 vol. %-95 vol. % |
| Fluorinated Ester 2 | tris(trimethylsilyl)phosphite (TTSPi) | 0.1 vol. %-5 vol. % |
| Fluorinated Ester 2 | tris(trimethylsilyl)phosphite (TTSPi) | >5 vol. %-25 vol. % |
| Fluorinated Ester 2 | tris(trimethylsilyl)phosphite (TTSPi) | >25 vol. %-95 vol. % |
| Fluorinated Ester 2 | tris(2,2,2-trifluoroethyl) phosphate | 0.1 vol. %-5 vol. % |
| | (TFEPa) | |
| Fluorinated Ester 2 | tris(2,2,2-trifluoroethyl) phosphate | >5 vol. %-25 vol. % |
| | (TFEPa) | |
| Fluorinated Ester 2 | tris(2,2,2-trifluoroethyl) phosphate | >25 vol. %-95 vol. % |
| | (TFEPa) | |
| Fluorinated Ester 2 | tris(2,2,2-trifluoroethyl) phosphite | 0.1 vol. %-5 vol. % |
| | (TFEPi) | |
| Fluorinated Ester 2 | tris(2,2,2-trifluoroethyl) phosphite | >5 vol. %-25 vol. % |
| | (TFEPi) | |
| Fluorinated Ester 2 | tris(2,2,2-trifluoroethyl) phosphite | >25 vol. %-95 vol. % |
| | (TFEPi) | |
| Fluorinated Ester 2 | (pentafluorophenyl)diphenyl phosphine | 0.1 vol. %-5 vol. % |
| | (PFPDPP) | |
| Fluorinated Ester 2 | (pentafluorophenyl)diphenyl phosphine | >5 vol. %-25 vol. % |
| | (PFPDPP) | |
| Fluorinated Ester 2 | (pentafluorophenyl)diphenyl phosphine | >25 vol. %-95 vol. % |
| | (PFPDPP) | |
| Fluorinated Ester 2 | tris(pentafluorophenyl) phosphine | 0.1 vol. %-5 vol. % |
| | (TPFPP) | |
| Fluorinated Ester 2 | tris(pentafluorophenyl) phosphine | >5 vol. %-25 vol. % |
| | (TPFPP) | |
| Fluorinated Ester 2 | tris(pentafluorophenyl) phosphine | >25 vol. %-95 vol. % |
| | (TPFPP) | |
| Fluorinated Ester 2 | 1,3,2-dioxathiolane-2,2-dioxide (DTD) | 0.1 vol. %-5 vol. % |
| Fluorinated Ester 2 | 1,3,2-dioxathiolane-2,2-dioxide (DTD) | >5 vol. %-25 vol. % |
| Fluorinated Ester 2 | 1,3,2-dioxathiolane-2,2-dioxide (DTD) | >25 vol. %-95 vol. % |
| Fluorinated Ester 2 | 1,3-propanesultone (PS) | 0.1 vol. %-5 vol. % |
| Fluorinated Ester 2 | 1,3-propanesultone (PS) | >5 vol. %-25 vol. % |
| Fluorinated Ester 2 | 1,3-propanesultone (PS) | >25 vol. %-95 vol. % |
| Fluorinated Ester 2 | prop-1-ene-1,3-sultone (PES) | 0.1 vol. %-5 vol. % |
| Fluorinated Ester 2 | prop-1-ene-1,3-sultone (PES) | >5 vol. %-25 vol. % |
| Fluorinated Ester 2 | prop-1-ene-1,3-sultone (PES) | >25 vol. %-95 vol. % |
| Fluorinated Ester 2 | propanediol cyclic sulfate (PCS) | 0.1 vol. %-5 vol. % |
| Fluorinated Ester 2 | propanediol cyclic sulfate (PCS) | >5 vol. %-25 vol. % |
| Fluorinated Ester 2 | propanediol cyclic sulfate (PCS) | >25 vol. %-95 vol. % |
| Fluorinated Ester 2 | 1,3,2-dioxathiolane 2-oxide | 0.1 vol. %-5 vol. % |
| Fluorinated Ester 2 | 1,3,2-dioxathiolane 2-oxide | >5 vol. %-25 vol. % |
| Fluorinated Ester 2 | 1,3,2-dioxathiolane 2-oxide | >25 vol. %-95 vol. % |
| Fluorinated Ester 2 | bis(2,2,2-trifluoroethyl) ether (BTFE) | 0.1 vol. %-5 vol. % |
| Fluorinated Ester 2 | bis(2,2,2-trifluoroethyl) ether (BTFE) | >5 vol. %-25 vol. % |
| Fluorinated Ester 2 | bis(2,2,2-trifluoroethyl) ether (BTFE) | >25 vol. %-95 vol. % |
| Fluorinated Ester 2 | 1,1,2,2-tetrafluoroethyl-2,2,3,3- | 0.1 vol. %-5 vol. % |
| | tetrafluoropropyl ether (TTE) | |
| Fluorinated Ester 2 | 1,1,2,2-tetrafluoroethyl-2,2,3,3- | >5 vol. %-25 vol. % |
| | tetrafluoropropyl ether (TTE) | |
| Fluorinated Ester 2 | 1,1,2,2-tetrafluoroethyl-2,2,3,3- | >25 vol. %-95 vol. % |
| | tetrafluoropropyl ether (TTE) | |
| Fluorinated Ester 2 | 1H,1H,5H-octafluoropentyl-1,1,2,2- | 0.1 vol. %-5 vol. % |
| | tetrafluoroethylether (OTE) | |
| Fluorinated Ester 2 | 1H,1H,5H-octafluoropentyl-1,1,2,2- | >5 vol. %-25 vol. % |
| | tetrafluoroethylether (OTE) | |
| Fluorinated Ester 2 | 1H,1H,5H-octafluoropentyl-1,1,2,2- | >25 vol. %-95 vol. % |
| | tetrafluoroethylether (OTE) | |
| Fluorinated Ester 2 | 2,2,2-trifluoroethyl 1,1,2,2- | 0.1 vol. %-5 vol. % |
| | tetrafluoroethyl ether (HFE) | |
| Fluorinated Ester 2 | 2,2,2-trifluoroethyl 1,1,2,2- | >5 vol. %-25 vol. % |
| | tetrafluoroethyl ether (HFE) | |
| Fluorinated Ester 2 | 2,2,2-trifluoroethyl 1,1,2,2- | >25 vol. %-95 vol. % |
| | tetrafluoroethyl ether (HFE) | |
| Fluorinated Ester 2 | tetrahydrofuran (THF) | 0.1 vol. %-5 vol. % |
| Fluorinated Ester 2 | tetrahydrofuran (THF) | >5 vol. %-25 vol. % |
| Fluorinated Ester 2 | tetrahydrofuran (THF) | >25 vol. %-95 vol. % |
| Fluorinated Ester 2 | 1,3-dioxolane (DOL) | 0.1 vol. %-5 vol. % |
| Fluorinated Ester 2 | 1,3-dioxolane (DOL) | >5 vol. %-25 vol. % |
| Fluorinated Ester 2 | 1,3-dioxolane (DOL) | >25 vol. %-95 vol. % |
| Fluorinated Ester 2 | 1,2-dimethoxyethane (DME) | 0.1 vol. %-5 vol. % |
| Fluorinated Ester 2 | 1,2-dimethoxyethane (DME) | >5 vol. %-25 vol. % |
| Fluorinated Ester 2 | 1,2-dimethoxyethane (DME) | >25 vol. %-95 vol. % |
| Fluorinated Ester 2 | diethylene glycol dimethyl ether | 0.1 vol. %-5 vol. % |
| | (DEGDME) | |
| Fluorinated Ester 2 | diethylene glycol dimethyl ether | >5 vol. %-25 vol. % |
| | (DEGDME) | |
| Fluorinated Ester 2 | diethylene glycol dimethyl ether | >25 vol. %-95 vol. % |
| | (DEGDME) | |
| Fluorinated Ester 2 | tetraethylene glycol dimethyl ether | 0.1 vol. %-5 vol. % |
| | (TEGDME) | |
| Fluorinated Ester 2 | tetraethylene glycol dimethyl ether | >5 vol. %-25 vol. % |
| | (TEGDME) | |
| Fluorinated Ester 2 | tetraethylene glycol dimethyl ether | >25 vol. %-95 vol. % |
| | (TEGDME) | |
| Fluorinated Ester 2 | succinic anhydride (SA) | 0.1 vol. %-5 vol. % |
| Fluorinated Ester 2 | succinic anhydride (SA) | >5 vol. %-25 vol. % |
| Fluorinated Ester 2 | succinic anhydride (SA) | >25 vol. %-95 vol. % |
| Fluorinated Ester 2 | butyric anhydride (BA) | 0.1 vol. %-5 vol. % |
| Fluorinated Ester 2 | butyric anhydride (BA) | >5 vol. %-25 vol. % |
| Fluorinated Ester 2 | butyric anhydride (BA) | >25 vol. %-95 vol. % |
| Fluorinated Ester 2 | 1,4-dioxane (DOX) | 0.1 vol. %-5 vol. % |
| Fluorinated Ester 2 | 1,4-dioxane (DOX) | >5 vol. %-25 vol. % |
| Fluorinated Ester 2 | 1,4-dioxane (DOX) | >25 vol. %-95 vol. % |
| Fluorinated Ester 2 | tetravinyl silane (TVSI) | 0.1 vol. %-5 vol. % |
| Fluorinated Ester 2 | tetravinyl silane (TVSI) | >5 vol. %-25 vol. % |
| Fluorinated Ester 2 | tetravinyl silane (TVSI) | >25 vol. %-95 vol. % |
| Fluorinated Ester 2 | 1,4-butane sultone (BS) | 0.1 vol. %-5 vol. % |
| Fluorinated Ester 2 | 1,4-butane sultone (BS) | >5 vol. %-25 vol. % |
| Fluorinated Ester 2 | 1,4-butane sultone (BS) | >25 vol. %-95 vol. % |
| Fluorinated Ester 2 | dimethyl sulfoxide (DMSO) | 0.1 vol. %-5 vol. % |
| Fluorinated Ester 2 | dimethyl sulfoxide (DMSO) | >5 vol. %-25 vol. % |
| Fluorinated Ester 2 | dimethyl sulfoxide (DMSO) | >25 vol. %-95 vol. % |
| Fluorinated Ester 2 | methylene methanedisulfonate | 0.1 vol. %-5 vol. % |
| | (MMDS) | |
| Fluorinated Ester 2 | methylene methanedisulfonate | >5 vol. %-25 vol. % |
| | (MMDS) | |
| Fluorinated Ester 2 | methylene methanedisulfonate | >25 vol. %-95 vol. % |
| | (MMDS) | |
| Fluorinated Ester 2 | N,N-dimethylformamide (DMF) | 0.1 vol. %-5 vol. % |
| Fluorinated Ester 2 | N,N-dimethylformamide (DMF) | >5 vol. %-25 vol. % |
| Fluorinated Ester 2 | N,N-dimethylformamide (DMF) | >25 vol. %-95 vol. % |
| Fluorinated Ester 2 | gamma-butyrolactone (BL) | 0.1 vol. %-5 vol. % |
| Fluorinated Ester 2 | gamma-butyrolactone (BL) | >5 vol. %-25 vol. % |
| Fluorinated Ester 2 | gamma-butyrolactone (BL) | >25 vol. %-95 vol. % |

TABLE 9F

| | | Concentration of |
|---|---|---|
| Fluorinated Ester | Solvent | Fluorinated Ester 3 |
| Fluorinated Ester 3 | fluoroethylene carbonate (FEC) | 0.1 vol. %-5 vol. % |
| Fluorinated Ester 3 | fluoroethylene carbonate (FEC) | >5 vol. %-25 vol. % |
| Fluorinated Ester 3 | fluoroethylene carbonate (FEC) | >25 vol. %-95 vol. % |
| Fluorinated Ester 3 | propylene carbonate (PC) | 0.1 vol. %-5 vol. % |
| Fluorinated Ester 3 | propylene carbonate (PC) | >5 vol. %-25 vol. % |
| Fluorinated Ester 3 | propylene carbonate (PC) | >25 vol. %-95 vol. % |
| Fluorinated Ester 3 | dimethyl carbonate (DMC) | 0.1 vol. %-5 vol. % |
| Fluorinated Ester 3 | dimethyl carbonate (DMC) | >5 vol. %-25 vol. % |
| Fluorinated Ester 3 | dimethyl carbonate (DMC) | >25 vol. %-95 vol. % |
| Fluorinated Ester 3 | diethyl carbonate (DEC) | 0.1 vol. %-5 vol. % |
| Fluorinated Ester 3 | diethyl carbonate (DEC) | >5 vol. %-25 vol. % |
| Fluorinated Ester 3 | diethyl carbonate (DEC) | >25 vol. %-95 vol. % |
| Fluorinated Ester 3 | ethyl methyl carbonate (EMC) | 0.1 vol. %-5 vol. % |
| Fluorinated Ester 3 | ethyl methyl carbonate (EMC) | >5 vol. %-25 vol. % |
| Fluorinated Ester 3 | ethyl methyl carbonate (EMC) | >25 vol. %-95 vol. % |
| Fluorinated Ester 3 | vinyl carbonate (VC) | 0.1 vol. %-5 vol. % |
| Fluorinated Ester 3 | vinyl carbonate (VC) | >5 vol. %-25 vol. % |
| Fluorinated Ester 3 | vinyl carbonate (VC) | >25 vol. %-95 vol. % |
| Fluorinated Ester 3 | vinyl ethylene carbonate (VEC) | 0.1 vol. %-5 vol. % |
| Fluorinated Ester 3 | vinyl ethylene carbonate (VEC) | >5 vol. %-25 vol. % |
| Fluorinated Ester 3 | vinyl ethylene carbonate (VEC) | >25 vol. %-95 vol. % |
| Fluorinated Ester 3 | ethylene carbonate (EC) | 0.1 vol. %-5 vol. % |
| Fluorinated Ester 3 | ethylene carbonate (EC) | >5 vol. %-25 vol. % |
| Fluorinated Ester 3 | ethylene carbonate (EC) | >25 vol. %-95 vol. % |
| Fluorinated Ester 3 | difluoroethylene carbonate (DFEC) | 0.1 vol. %-5 vol. % |
| Fluorinated Ester 3 | difluoroethylene carbonate (DFEC) | >5 vol. %-25 vol. % |
| Fluorinated Ester 3 | difluoroethylene carbonate (DFEC) | >25 vol. %-95 vol. % |
| Fluorinated Ester 3 | 3,3,3-trifluoropropylene carbonate | 0.1 vol. %-5 vol. % |
| | (TFPC) | |
| Fluorinated Ester 3 | 3,3,3-trifluoropropylene carbonate | >5 vol. %-25 vol. % |
| | (TFPC) | |
| Fluorinated Ester 3 | 3,3,3-trifluoropropylene carbonate | >25 vol. %-95 vol. % |
| | (TFPC) | |
| Fluorinated Ester 3 | monofluoroethyl methyl carbonate | 0.1 vol. %-5 vol. % |
| | (F1EMC) | |
| Fluorinated Ester 3 | monofluoroethyl methyl carbonate | >5 vol. %-25 vol. % |
| | (F1EMC) | |
| Fluorinated Ester 3 | monofluoroethyl methyl carbonate | >25 vol. %-95 vol. % |
| | (F1EMC) | |
| Fluorinated Ester 3 | difluoroethyl methyl carbonate | 0.1 vol. %-5 vol. % |
| | (F2EMC) | |
| Fluorinated Ester 3 | difluoroethyl methyl carbonate | >5 vol. %-25 vol. % |
| | (F2EMC) | |
| Fluorinated Ester 3 | difluoroethyl methyl carbonate | >25 vol. %-95 vol. % |
| | (F2EMC) | |
| Fluorinated Ester 3 | trifluoroethyl methyl carbonate | 0.1 vol. %-5 vol. % |
| | (F3EMC) | |
| Fluorinated Ester 3 | trifluoroethyl methyl carbonate | >5 vol. %-25 vol. % |
| | (F3EMC) | |
| Fluorinated Ester 3 | trifluoroethyl methyl carbonate | >25 vol. %-95 vol. % |
| | (F3EMC) | |
| Fluorinated Ester 3 | bis(2,2,2-trifluoroethyl) carbonate | 0.1 vol. %-5 vol. % |
| | (TFEC) | |
| Fluorinated Ester 3 | bis(2,2,2-trifluoroethyl) carbonate | >5 vol. %-25 vol. % |
| | (TFEC) | |
| Fluorinated Ester 3 | bis(2,2,2-trifluoroethyl) carbonate | >25 vol. %-95 vol. % |
| | (TFEC) | |
| Fluorinated Ester 3 | 2,2-difluoroethyl ethyl carbonate | 0.1 vol. %-5 vol. % |
| | (DFDEC) | |
| Fluorinated Ester 3 | 2,2-difluoroethyl ethyl carbonate | >5 vol. %-25 vol. % |
| | (DFDEC) | |
| Fluorinated Ester 3 | 2,2-difluoroethyl ethyl carbonate | >25 vol. %-95 vol. % |
| | (DFDEC) | |
| Fluorinated Ester 3 | acetonitrile (AN) | 0.1 vol. %-5 vol. % |
| Fluorinated Ester 3 | acetonitrile (AN) | >5 vol. %-25 vol. % |
| Fluorinated Ester 3 | acetonitrile (AN) | >25 vol. %-95 vol. % |
| Fluorinated Ester 3 | fluoroacetonitrile | 0.1 vol. %-5 vol. % |
| Fluorinated Ester 3 | fluoroacetonitrile | >5 vol. %-25 vol. % |
| Fluorinated Ester 3 | fluoroacetonitrile | >25 vol. %-95 vol. % |
| Fluorinated Ester 3 | ethyl acetate (EA) | 0.1 vol. %-5 vol. % |
| Fluorinated Ester 3 | ethyl acetate (EA) | >5 vol. %-25 vol. % |
| Fluorinated Ester 3 | ethyl acetate (EA) | >25 vol. %-95 vol. % |
| Fluorinated Ester 3 | methyl acetate (MA) | 0.1 vol. %-5 vol. % |
| Fluorinated Ester 3 | methyl acetate (MA) | >5 vol. %-25 vol. % |
| Fluorinated Ester 3 | methyl acetate (MA) | >25 vol. %-95 vol. % |
| Fluorinated Ester 3 | methyl propanoate (MP) | 0.1 vol. %-5 vol. % |
| Fluorinated Ester 3 | methyl propanoate (MP) | >5 vol. %-25 vol. % |
| Fluorinated Ester 3 | methyl propanoate (MP) | >25 vol. %-95 vol. % |
| Fluorinated Ester 3 | methyl 3,3,3-trifluoropropanoate | 0.1 vol. %-5 vol. % |
| Fluorinated Ester 3 | methyl 3,3,3-trifluoropropanoate | >5 vol. %-25 vol. % |
| Fluorinated Ester 3 | methyl 3,3,3-trifluoropropanoate | >25 vol. %-95 vol. % |
| Fluorinated Ester 3 | ethyl 3,3,3-trifluoropropanoate | 0.1 vol. %-5 vol. % |
| Fluorinated Ester 3 | ethyl 3,3,3-trifluoropropanoate | >5 vol. %-25 vol. % |
| Fluorinated Ester 3 | ethyl 3,3,3-trifluoropropanoate | >25 vol. %-95 vol. % |
| Fluorinated Ester 3 | ethyl 2,2-difluoropropanoate | 0.1 vol. %-5 vol. % |
| Fluorinated Ester 3 | ethyl 2,2-difluoropropanoate | >5 vol. %-25 vol. % |
| Fluorinated Ester 3 | ethyl 2,2-difluoropropanoate | >25 vol. %-95 vol. % |
| Fluorinated Ester 3 | isopropyl trifluoroacetate | 0.1 vol. %-5 vol. % |
| Fluorinated Ester 3 | isopropyl trifluoroacetate | >5 vol. %-25 vol. % |
| Fluorinated Ester 3 | isopropyl trifluoroacetate | >25 vol. %-95 vol. % |
| Fluorinated Ester 3 | methyl 4,4,4-trifluorobutanoate | 0.1 vol. %-5 vol. % |
| Fluorinated Ester 3 | methyl 4,4,4-trifluorobutanoate | >5 vol. %-25 vol. % |
| Fluorinated Ester 3 | methyl 4,4,4-trifluorobutanoate | >25 vol. %-95 vol. % |
| Fluorinated Ester 3 | methyl difluoroacetate | 0.1 vol. %-5 vol. % |
| Fluorinated Ester 3 | methyl difluoroacetate | >5 vol. %-25 vol. % |
| Fluorinated Ester 3 | methyl difluoroacetate | >25 vol. %-95 vol. % |
| Fluorinated Ester 3 | 2,2,2-trifluoroethyl butanoate | 0.1 vol. %-5 vol. % |
| Fluorinated Ester 3 | 2,2,2-trifluoroethyl butanoate | >5 vol. %-25 vol. % |
| Fluorinated Ester 3 | 2,2,2-trifluoroethyl butanoate | >25 vol. %-95 vol. % |
| Fluorinated Ester 3 | methyl 2,2-difluoropropanoate | 0.1 vol. %-5 vol. % |
| Fluorinated Ester 3 | methyl 2,2-difluoropropanoate | >5 vol. %-25 vol. % |
| Fluorinated Ester 3 | methyl 2,2-difluoropropanoate | >25 vol. %-95 vol. % |
| Fluorinated Ester 3 | ethyl trifluoroacetate | 0.1 vol. %-5 vol. % |
| Fluorinated Ester 3 | ethyl trifluoroacetate | >5 vol. %-25 vol. % |
| Fluorinated Ester 3 | ethyl trifluoroacetate | >25 vol. %-95 vol. % |
| Fluorinated Ester 3 | 2,2-difluoroethyl trifluoroacetate | 0.1 vol. %-5 vol. % |
| Fluorinated Ester 3 | 2,2-difluoroethyl trifluoroacetate | >5 vol. %-25 vol. % |
| Fluorinated Ester 3 | 2,2-difluoroethyl trifluoroacetate | >25 vol. %-95 vol. % |
| Fluorinated Ester 3 | 2,2,2-trifluoroethyl acetate | 0.1 vol. %-5 vol. % |
| Fluorinated Ester 3 | 2,2,2-trifluoroethyl acetate | >5 vol. %-25 vol. % |
| Fluorinated Ester 3 | 2,2,2-trifluoroethyl acetate | >25 vol. %-95 vol. % |
| Fluorinated Ester 3 | ethyl 2,2-difluoroacetate | 0.1 vol. %-5 vol. % |
| Fluorinated Ester 3 | ethyl 2,2-difluoroacetate | >5 vol. %-25 vol. % |
| Fluorinated Ester 3 | ethyl 2,2-difluoroacetate | >25 vol. %-95 vol. % |
| Fluorinated Ester 3 | 2,2-difluoroethyl acetate | 0.1 vol. %-5 vol. % |
| Fluorinated Ester 3 | 2,2-difluoroethyl acetate | >5 vol. %-25 vol. % |
| Fluorinated Ester 3 | 2,2-difluoroethyl acetate | >25 vol. %-95 vol. % |
| Fluorinated Ester 3 | 2,2-difluoroethyl 3,3- | 0.1 vol. %-5 vol. % |
| | difluoropropanoate | |
| Fluorinated Ester 3 | 2,2-difluoroethyl 3,3- | >5 vol. %-25 vol. % |
| | difluoropropanoate | |
| Fluorinated Ester 3 | 2,2-difluoroethyl 3,3- | >25 vol. %-95 vol. % |
| | difluoropropanoate | |
| Fluorinated Ester 3 | 2,2-difluoroethyl 2,2-difluoroacetate | 0.1 vol. %-5 vol. % |
| Fluorinated Ester 3 | 2,2-difluoroethyl 2,2-difluoroacetate | >5 vol. %-25 vol. % |
| Fluorinated Ester 3 | 2,2-difluoroethyl 2,2-difluoroacetate | >25 vol. %-95 vol. % |
| Fluorinated Ester 3 | succinonitrile (SN) | 0.1 vol. %-5 vol. % |
| Fluorinated Ester 3 | succinonitrile (SN) | >5 vol. %-25 vol. % |
| Fluorinated Ester 3 | succinonitrile (SN) | >25 vol. %-95 vol. % |
| Fluorinated Ester 3 | propionitrile (PN) | 0.1 vol. %-5 vol. % |
| Fluorinated Ester 3 | propionitrile (PN) | >5 vol. %-25 vol. % |
| Fluorinated Ester 3 | propionitrile (PN) | >25 vol. %-95 vol. % |
| Fluorinated Ester 3 | butyronitrile (BN) | 0.1 vol. %-5 vol. % |
| Fluorinated Ester 3 | butyronitrile (BN) | >5 vol. %-25 vol. % |
| Fluorinated Ester 3 | butyronitrile (BN) | >25 vol. %-95 vol. % |
| Fluorinated Ester 3 | adiponitrile (ADN) | 0.1 vol. %-5 vol. % |
| Fluorinated Ester 3 | adiponitrile (ADN) | >5 vol. %-25 vol. % |
| Fluorinated Ester 3 | adiponitrile (ADN) | >25 vol. %-95 vol. % |
| Fluorinated Ester 3 | 1,3,6-hexanetricarbonitrile (HTCN) | 0.1 vol. %-5 vol. % |
| Fluorinated Ester 3 | 1,3,6-hexanetricarbonitrile (HTCN) | >5 vol. %-25 vol. % |
| Fluorinated Ester 3 | 1,3,6-hexanetricarbonitrile (HTCN) | >25 vol. %-95 vol. % |
| Fluorinated Ester 3 | trimethyl borate (TMB) | 0.1 vol. %-5 vol. % |
| Fluorinated Ester 3 | trimethyl borate (TMB) | >5 vol. %-25 vol. % |
| Fluorinated Ester 3 | trimethyl borate (TMB) | >25 vol. %-95 vol. % |
| Fluorinated Ester 3 | triphenyl borate (TPB) | 0.1 vol. %-5 vol. % |
| Fluorinated Ester 3 | triphenyl borate (TPB) | >5 vol. %-25 vol. % |
| Fluorinated Ester 3 | triphenyl borate (TPB) | >25 vol. %-95 vol. % |
| Fluorinated Ester 3 | triethyl borate (TEB) | 0.1 vol. %-5 vol. % |
| Fluorinated Ester 3 | triethyl borate (TEB) | >5 vol. %-25 vol. % |
| Fluorinated Ester 3 | triethyl borate (TEB) | >25 vol. %-95 vol. % |
| Fluorinated Ester 3 | tris(pentafluorophenyl)borane (TPFPB) | 0.1 vol. %-5 vol. % |
| Fluorinated Ester 3 | tris(pentafluorophenyl)borane (TPFPB) | >5 vol. %-25 vol. % |
| Fluorinated Ester 3 | tris(pentafluorophenyl)borane (TPFPB) | >25 vol. %-95 vol. % |
| Fluorinated Ester 3 | tris(trimethylsilyl)phosphate (TTSB) | 0.1 vol. %-5 vol. % |
| Fluorinated Ester 3 | tris(trimethylsilyl)phosphate (TTSB) | >5 vol. %-25 vol. % |
| Fluorinated Ester 3 | tris(trimethylsilyl)phosphate (TTSB) | >25 vol. %-95 vol. % |
| Fluorinated Ester 3 | tris(2,2,2-trifluoroethyl) borate (TTFEB) | 0.1 vol. %-5 vol. % |
| Fluorinated Ester 3 | tris(2,2,2-trifluoroethyl) borate (TTFEB) | >5 vol. %-25 vol. % |
| Fluorinated Ester 3 | tris(2,2,2-trifluoroethyl) borate (TTFEB) | >25 vol. %-95 vol. % |
| Fluorinated Ester 3 | trimethyl phosphate (TMP) | 0.1 vol. %-5 vol. % |
| Fluorinated Ester 3 | trimethyl phosphate (TMP) | >5 vol. %-25 vol. % |
| Fluorinated Ester 3 | trimethyl phosphate (TMP) | >25 vol. %-95 vol. % |
| Fluorinated Ester 3 | triethyl phosphate (TEP) | 0.1 vol. %-5 vol. % |
| Fluorinated Ester 3 | triethyl phosphate (TEP) | >5 vol. %-25 vol. % |
| Fluorinated Ester 3 | triethyl phosphate (TEP) | >25 vol. %-95 vol. % |
| Fluorinated Ester 3 | tris(trimethylsilyl)phosphate (TTSP) | 0.1 vol. %-5 vol. % |
| Fluorinated Ester 3 | tris(trimethylsilyl)phosphate (TTSP) | >5 vol. %-25 vol. % |
| Fluorinated Ester 3 | tris(trimethylsilyl)phosphate (TTSP) | >25 vol. %-95 vol. % |
| Fluorinated Ester 3 | tris(trimethylsilyl)phosphite (TTSPi) | 0.1 vol. %-5 vol. % |
| Fluorinated Ester 3 | tris(trimethylsilyl)phosphite (TTSPi) | >5 vol. %-25 vol. % |
| Fluorinated Ester 3 | tris(trimethylsilyl)phosphite (TTSPi) | >25 vol. %-95 vol. % |
| Fluorinated Ester 3 | tris(2,2,2-trifluoroethyl) phosphate | 0.1 vol. %-5 vol. % |
| | (TFEPa) | |
| Fluorinated Ester 3 | tris(2,2,2-trifluoroethyl) phosphate | >5 vol. %-25 vol. % |
| | (TFEPa) | |
| Fluorinated Ester 3 | tris(2,2,2-trifluoroethyl) phosphate | >25 vol. %-95 vol. % |
| | (TFEPa) | |
| Fluorinated Ester 3 | tris(2,2,2-trifluoroethyl) phosphite | 0.1 vol. %-5 vol. % |
| | (TFEPi) | |
| Fluorinated Ester 3 | tris(2,2,2-trifluoroethyl) phosphite | >5 vol. %-25 vol. % |
| | (TFEPi) | |
| Fluorinated Ester 3 | tris(2,2,2-trifluoroethyl) phosphite | >25 vol. %-95 vol. % |
| | (TFEPi) | |
| Fluorinated Ester 3 | (pentafluorophenyl)diphenyl phosphine | 0.1 vol. %-5 vol. % |
| | (PFPDPP) | |
| Fluorinated Ester 3 | (pentafluorophenyl)diphenyl phosphine | >5 vol. %-25 vol. % |
| | (PFPDPP) | |
| Fluorinated Ester 3 | (pentafluorophenyl)diphenyl phosphine | >25 vol. %-95 vol. % |
| | (PFPDPP) | |
| Fluorinated Ester 3 | tris(pentafluorophenyl) phosphine | 0.1 vol. %-5 vol. % |
| | (TPFPP) | |
| Fluorinated Ester 3 | tris(pentafluorophenyl) phosphine | >5 vol. %-25 vol. % |
| | (TPFPP) | |
| Fluorinated Ester 3 | tris(pentafluorophenyl) phosphine | >25 vol. %-95 vol. % |
| | (TPFPP) | |
| Fluorinated Ester 3 | 1,3,2-dioxathiolane-2,2-dioxide (DTD) | 0.1 vol. %-5 vol. % |
| Fluorinated Ester 3 | 1,3,2-dioxathiolane-2,2-dioxide (DTD) | >5 vol. %-25 vol. % |
| Fluorinated Ester 3 | 1,3,2-dioxathiolane-2,2-dioxide (DTD) | >25 vol. %-95 vol. % |
| Fluorinated Ester 3 | 1,3-propanesultone (PS) | 0.1 vol. %-5 vol. % |
| Fluorinated Ester 3 | 1,3-propanesultone (PS) | >5 vol. %-25 vol. % |
| Fluorinated Ester 3 | 1,3-propanesultone (PS) | >25 vol. %-95 vol. % |
| Fluorinated Ester 3 | prop-1-ene-1,3-sultone (PES) | 0.1 vol. %-5 vol. % |
| Fluorinated Ester 3 | prop-1-ene-1,3-sultone (PES) | >5 vol. %-25 vol. % |
| Fluorinated Ester 3 | prop-1-ene-1,3-sultone (PES) | >25 vol. %-95 vol. % |
| Fluorinated Ester 3 | propanediol cyclic sulfate (PCS) | 0.1 vol. %-5 vol. % |
| Fluorinated Ester 3 | propanediol cyclic sulfate (PCS) | >5 vol. %-25 vol. % |
| Fluorinated Ester 3 | propanediol cyclic sulfate (PCS) | >25 vol. %-95 vol. % |
| Fluorinated Ester 3 | 1,3,2-dioxathiolane 2-oxide | 0.1 vol. %-5 vol. % |
| Fluorinated Ester 3 | 1,3,2-dioxathiolane 2-oxide | >5 vol. %-25 vol. % |
| Fluorinated Ester 3 | 1,3,2-dioxathiolane 2-oxide | >25 vol. %-95 vol. % |
| Fluorinated Ester 3 | bis(2,2,2-trifluoroethyl) ether (BTFE) | 0.1 vol. %-5 vol. % |
| Fluorinated Ester 3 | bis(2,2,2-trifluoroethyl) ether (BTFE) | >5 vol. %-25 vol. % |
| Fluorinated Ester 3 | bis(2,2,2-trifluoroethyl) ether (BTFE) | >25 vol. %-95 vol. % |
| Fluorinated Ester 3 | 1,1,2,2-tetrafluoroethyl-2,2,3,3- | 0.1 vol. %-5 vol. % |
| | tetrafluoropropyl ether (TTE) | |
| Fluorinated Ester 3 | 1,1,2,2-tetrafluoroethyl-2,2,3,3- | >5 vol. %-25 vol. % |
| | tetrafluoropropyl ether (TTE) | |
| Fluorinated Ester 3 | 1,1,2,2-tetrafluoroethyl-2,2,3,3- | >25 vol. %-95 vol. % |
| | tetrafluoropropyl ether (TTE) | |
| Fluorinated Ester 3 | 1H,1H,5H-octafluoropentyl-1,1,2,2- | 0.1 vol. %-5 vol. % |
| | tetrafluoroethylether (OTE) | |
| Fluorinated Ester 3 | 1H,1H,5H-octafluoropentyl-1,1,2,2- | >5 vol. %-25 vol. % |
| | tetrafluoroethylether (OTE) | |
| Fluorinated Ester 3 | 1H,1H,5H-octafluoropentyl-1,1,2,2- | >25 vol. %-95 vol. % |
| | tetrafluoroethylether (OTE) | |
| Fluorinated Ester 3 | 2,2,2-trifluoroethyl 1,1,2,2- | 0.1 vol. %-5 vol. % |
| | tetrafluoroethyl ether (HFE) | |
| Fluorinated Ester 3 | 2,2,2-trifluoroethyl 1,1,2,2- | >5 vol. %-25 vol. % |
| | tetrafluoroethyl ether (HFE) | |
| Fluorinated Ester 3 | 2,2,2-trifluoroethyl 1,1,2,2- | >25 vol. %-95 vol. % |
| | tetrafluoroethyl ether (HFE) | |
| Fluorinated Ester 3 | tetrahydrofuran (THF) | 0.1 vol. %-5 vol. % |
| Fluorinated Ester 3 | tetrahydrofuran (THF) | >5 vol. %-25 vol. % |
| Fluorinated Ester 3 | tetrahydrofuran (THF) | >25 vol. %-95 vol. % |
| Fluorinated Ester 3 | 1,3-dioxolane (DOL) | 0.1 vol. %-5 vol. % |
| Fluorinated Ester 3 | 1,3-dioxolane (DOL) | >5 vol. %-25 vol. % |
| Fluorinated Ester 3 | 1,3-dioxolane (DOL) | >25 vol. %-95 vol. % |
| Fluorinated Ester 3 | 1,2-dimethoxyethane (DME) | 0.1 vol. %-5 vol. % |
| Fluorinated Ester 3 | 1,2-dimethoxyethane (DME) | >5 vol. %-25 vol. % |
| Fluorinated Ester 3 | 1,2-dimethoxyethane (DME) | >25 vol. %-95 vol. % |
| Fluorinated Ester 3 | diethylene glycol dimethyl ether | 0.1 vol. %-5 vol. % |
| | (DEGDME) | |
| Fluorinated Ester 3 | diethylene glycol dimethyl ether | >5 vol. %-25 vol. % |
| | (DEGDME) | |
| Fluorinated Ester 3 | diethylene glycol dimethyl ether | >25 vol. %-95 vol. % |
| | (DEGDME) | |
| Fluorinated Ester 3 | tetraethylene glycol dimethyl ether | 0.1 vol. %-5 vol. % |
| | (TEGDME) | |
| Fluorinated Ester 3 | tetraethylene glycol dimethyl ether | >5 vol. %-25 vol. % |
| | (TEGDME) | |
| Fluorinated Ester 3 | tetraethylene glycol dimethyl ether | >25 vol. %-95 vol. % |
| | (TEGDME) | |
| Fluorinated Ester 3 | succinic anhydride (SA) | 0.1 vol. %-5 vol. % |
| Fluorinated Ester 3 | succinic anhydride (SA) | >5 vol. %-25 vol. % |
| Fluorinated Ester 3 | succinic anhydride (SA) | >25 vol. %-95 vol. % |
| Fluorinated Ester 3 | butyric anhydride (BA) | 0.1 vol. %-5 vol. % |
| Fluorinated Ester 3 | butyric anhydride (BA) | >5 vol. %-25 vol. % |
| Fluorinated Ester 3 | butyric anhydride (BA) | >25 vol. %-95 vol. % |
| Fluorinated Ester 3 | 1,4-dioxane (DOX) | 0.1 vol. %-5 vol. % |
| Fluorinated Ester 3 | 1,4-dioxane (DOX) | >5 vol. %-25 vol. % |
| Fluorinated Ester 3 | 1,4-dioxane (DOX) | >25 vol. %-95 vol. % |
| Fluorinated Ester 3 | tetravinyl silane (TVSI) | 0.1 vol. %-5 vol. % |
| Fluorinated Ester 3 | tetravinyl silane (TVSI) | >5 vol. %-25 vol. % |
| Fluorinated Ester 3 | tetravinyl silane (TVSI) | >25 vol. %-95 vol. % |
| Fluorinated Ester 3 | 1,4-butane sultone (BS) | 0.1 vol. %-5 vol. % |
| Fluorinated Ester 3 | 1,4-butane sultone (BS) | >5 vol. %-25 vol. % |
| Fluorinated Ester 3 | 1,4-butane sultone (BS) | >25 vol. %-95 vol. % |
| Fluorinated Ester 3 | dimethyl sulfoxide (DMSO) | 0.1 vol. %-5 vol. % |
| Fluorinated Ester 3 | dimethyl sulfoxide (DMSO) | >5 vol. %-25 vol. % |
| Fluorinated Ester 3 | dimethyl sulfoxide (DMSO) | >25 vol. %-95 vol. % |
| Fluorinated Ester 3 | methylene methanedisulfonate | 0.1 vol. %-5 vol. % |
| | (MMDS) | |
| Fluorinated Ester 3 | methylene methanedisulfonate | >5 vol. %-25 vol. % |
| | (MMDS) | |
| Fluorinated Ester 3 | methylene methanedisulfonate | >25 vol. %-95 vol. % |
| | (MMDS) | |
| Fluorinated Ester 3 | N,N-dimethylformamide (DMF) | 0.1 vol. %-5 vol. % |
| Fluorinated Ester 3 | N,N-dimethylformamide (DMF) | >5 vol. %-25 vol. % |
| Fluorinated Ester 3 | N,N-dimethylformamide (DMF) | >25 vol. %-95 vol. % |
| Fluorinated Ester 3 | gamma-butyrolactone (BL) | 0.1 vol. %-5 vol. % |
| Fluorinated Ester 3 | gamma-butyrolactone (BL) | >5 vol. %-25 vol. % |
| Fluorinated Ester 3 | gamma-butyrolactone (BL) | >25 vol. %-95 vol. % |

An electrolyte including at least one of Fluorinated Esters 1-3 is prepared by dissolving at least one of Lithium Salt 1-16 (for formation of a lithium-ion battery) or at least one of Sodium Salt 1-13 (for formation of a sodium-ion battery) at a concentration of 0.01 M-5 M in a solvent mixture of at least one of Fluorinated Esters 1-3 and optionally at least one supplemental solvent from Tables 9A-9F. The amount of the fluorinated ester in the electrolyte is 0.1-95 wt. % and the amount of the at least one supplemental solvent in the electrolyte is 0-99 wt. % or 0.1-99 wt. % respectively, based on a total weight of the electrolyte. The amount of the fluorinated ester in the electrolyte is 0.1-95 vol % and the amount of the at least one supplemental solvent in the electrolyte is 0-99 vol. % or 0.1-99 vol. %, respectively, based on a total volume of the electrolyte. Dissolving can be aided by application of heat, agitation, or a combination thereof.

### C. Additives

The electrolyte can further include one or more additives in addition to at least one of Fluorinated Esters 1-3 and a metal salt. The additive can be, for example, a conductive additive, an overcharge protection additive, a flame-retardant additive, a solid electrolyte interphase (SEI) film-forming additive, a cathode electrolyte interphase (CEI) film forming additive, a cathode material protective agent, metal salt stabilizer, or a combination thereof.

### V. Battery Formation

The present disclosure provides batteries and rechargeable batteries including electrolytes, which include at least one of Fluorinated Esters 1-3, an anode, a cathode, and a separator. An exemplary rechargeable battery is illustrated in the FIGURE, which shows a cathode, an anode, a porous separator and electrolyte of the present disclosure which facilitates the flow of ions, for example, lithium or sodium ions, between the cathode and the anode.

### A. Anode

Table 10 provides anode materials that can be used in a battery including an electrolyte including at least one lithium salt and at least one of Fluorinated Esters 1-3. Table 11 provides anode materials that can be used in a battery including an electrolyte including at least one sodium salt and at least one of Fluorinated Esters 1-3. The anode material can be selected depending on a battery being formed and uses thereof. It is understood that the invention comprises, consists essentially of, or consists of the compositions identified in Table 10. It is understood that the invention comprises, consists essentially of, or consists of the compositions identified in Table 11.

TABLE 10

| Fluorinated Ester | Anode Material |
|---|---|
| Fluorinated Ester 1 | graphite |
| Fluorinated Ester 1 | lithium metal |
| Fluorinated Ester 1 | silicon |
| Fluorinated Ester 1 | silicon oxide (SiOₓ) |
| Fluorinated Ester 1 | graphite/silicon composite |
| Fluorinated Ester 1 | graphite/silicon oxide composite |
| Fluorinated Ester 1 | silicon/carbon composite |
| Fluorinated Ester 1 | silicon carbide |
| Fluorinated Ester 1 | graphite/silicon carbide composite |
| Fluorinated Ester 1 | silicon nitride |
| Fluorinated Ester 1 | graphite/silicon nitride composite |
| Fluorinated Ester 1 | hard carbon |
| Fluorinated Ester 1 | amorphous carbon |
| Fluorinated Ester 1 | lithium titanium oxide (LTO) |
| Fluorinated Ester 2 | graphite |
| Fluorinated Ester 2 | lithium metal |
| Fluorinated Ester 2 | silicon |
| Fluorinated Ester 2 | silicon oxide (SiOₓ) |
| Fluorinated Ester 2 | graphite/silicon composite |
| Fluorinated Ester 2 | graphite/silicon oxide composite |
| Fluorinated Ester 2 | silicon/carbon composite |
| Fluorinated Ester 2 | silicon carbide |
| Fluorinated Ester 2 | graphite/silicon carbide composite |
| Fluorinated Ester 2 | silicon nitride |
| Fluorinated Ester 2 | graphite/silicon nitride composite |
| Fluorinated Ester 2 | hard carbon |
| Fluorinated Ester 2 | amorphous carbon |
| Fluorinated Ester 2 | lithium titanium oxide (LTO) |
| Fluorinated Ester 3 | graphite |
| Fluorinated Ester 3 | lithium metal |
| Fluorinated Ester 3 | silicon |
| Fluorinated Ester 3 | silicon oxide (SiOₓ) |
| Fluorinated Ester 3 | graphite/silicon composite |
| Fluorinated Ester 3 | graphite/silicon oxide composite |
| Fluorinated Ester 3 | silicon/carbon composite |
| Fluorinated Ester 3 | silicon carbide |
| Fluorinated Ester 3 | graphite/silicon carbide composite |
| Fluorinated Ester 3 | silicon nitride |
| Fluorinated Ester 3 | graphite/silicon nitride composite |
| Fluorinated Ester 3 | hard carbon |
| Fluorinated Ester 3 | amorphous carbon |
| Fluorinated Ester 3 | lithium titanium oxide (LTO) |

TABLE 11

| Fluorinated Ester | Anode Material |
|---|---|
| Fluorinated Ester 1 | sodium metal |
| Fluorinated Ester 1 | graphite |
| Fluorinated Ester 1 | silicon |
| Fluorinated Ester 1 | silicon oxide (SiOₓ) |
| Fluorinated Ester 1 | graphite/silicon composite |
| Fluorinated Ester 1 | graphite/silicon oxide composite |
| Fluorinated Ester 1 | silicon/carbon composite |
| Fluorinated Ester 1 | silicon carbide |
| Fluorinated Ester 1 | graphite/silicon carbide composite |
| Fluorinated Ester 1 | silicon nitride |
| Fluorinated Ester 1 | graphite/silicon nitride composite |
| Fluorinated Ester 1 | hard carbon |
| Fluorinated Ester 1 | amorphous carbon |
| Fluorinated Ester 1 | sodium titanium oxide (NTO) |
| Fluorinated Ester 1 | tin |
| Fluorinated Ester 1 | tin oxide |
| Fluorinated Ester 1 | tin sulfide |
| Fluorinated Ester 2 | sodium metal |
| Fluorinated Ester 2 | graphite |
| Fluorinated Ester 2 | silicon |
| Fluorinated Ester 2 | silicon oxide (SiOₓ) |
| Fluorinated Ester 2 | graphite/silicon composite |
| Fluorinated Ester 2 | graphite/silicon oxide composite |
| Fluorinated Ester 2 | silicon/carbon composite |
| Fluorinated Ester 2 | silicon carbide |
| Fluorinated Ester 2 | graphite/silicon carbide composite |
| Fluorinated Ester 2 | silicon nitride |
| Fluorinated Ester 2 | graphite/silicon nitride composite |
| Fluorinated Ester 2 | hard carbon |
| Fluorinated Ester 2 | amorphous carbon |
| Fluorinated Ester 2 | sodium titanium oxide (NTO) |
| Fluorinated Ester 2 | tin |
| Fluorinated Ester 2 | tin oxide |
| Fluorinated Ester 2 | tin sulfide |
| Fluorinated Ester 3 | sodium metal |
| Fluorinated Ester 3 | graphite |
| Fluorinated Ester 3 | silicon |
| Fluorinated Ester 3 | silicon oxide (SiOₓ) |
| Fluorinated Ester 3 | graphite/silicon composite |
| Fluorinated Ester 3 | graphite/silicon oxide composite |
| Fluorinated Ester 3 | silicon/carbon composite |
| Fluorinated Ester 3 | silicon carbide |
| Fluorinated Ester 3 | graphite/silicon carbide composite |
| Fluorinated Ester 3 | silicon nitride |
| Fluorinated Ester 3 | graphite/silicon nitride composite |
| Fluorinated Ester 3 | hard carbon |
| Fluorinated Ester 3 | amorphous carbon |
| Fluorinated Ester 3 | sodium titanium oxide (NTO) |
| Fluorinated Ester 3 | tin |
| Fluorinated Ester 3 | tin oxide |
| Fluorinated Ester 3 | tin sulfide |

### B. Cathode

Table 12 provides cathode materials that can be used in a battery including an electrolyte including at least one lithium salt and at least one of Fluorinated Esters 1-3. Table 13 provides cathode materials that can be used in a battery including an electrolyte including at least one sodium salt and at least one of Fluorinated Esters 1-3. The cathode material can be selected depending on a battery being formed and uses thereof. It is understood that the invention comprises, consists essentially of, or consists of the compositions identified in Table 12. It is understood that the invention comprises, consists essentially of, or consists of the compositions identified in Table 13.

TABLE 12

| Fluorinated Ester | Cathode Material |
|---|---|
| Fluorinated Ester 1 | lithium nickel manganese cobalt oxide (NMC) |
| Fluorinated Ester 1 | lithium nickel cobalt aluminum oxide (NCA) |
| Fluorinated Ester 1 | lithium nickel manganese aluminum oxide (NMA) |
| Fluorinated Ester 1 | lithium nickel manganese cobalt aluminum oxide (NMCA) |
| Fluorinated Ester 1 | lithium nickel oxide (LNO) |
| Fluorinated Ester 1 | lithium nickel manganese oxide (LiNi_{0.5}Mn_{1.5}O₄) |
| Fluorinated Ester 1 | lithium cobalt oxide (LCO) |
| Fluorinated Ester 1 | lithium manganese oxide (LMO) |
| Fluorinated Ester 1 | lithium and manganese-rich cathode (LMR or LLMO) |
| Fluorinated Ester 1 | lithium iron phosphate (LFP) |
| Fluorinated Ester 1 | lithium cobalt phosphate (LCP) |
| Fluorinated Ester 1 | lithium manganese phosphate (LMP) |
| Fluorinated Ester 1 | lithium manganese iron phosphate (LMFP) |
| Fluorinated Ester 1 | transition metal sulfide |
| Fluorinated Ester 1 | sulfur |
| Fluorinated Ester 1 | sulfur/carbon composite |
| Fluorinated Ester 2 | lithium nickel manganese cobalt oxide (NMC) |
| Fluorinated Ester 2 | lithium nickel cobalt aluminum oxide (NCA) |
| Fluorinated Ester 2 | lithium nickel manganese aluminum oxide (NMA) |
| Fluorinated Ester 2 | lithium nickel manganese cobalt aluminum oxide (NMCA) |
| Fluorinated Ester 2 | lithium nickel oxide (LNO) |
| Fluorinated Ester 2 | lithium nickel manganese oxide (LiNi_{0.5}Mn_{1.5}O₄) |
| Fluorinated Ester 2 | lithium cobalt oxide (LCO) |
| Fluorinated Ester 2 | lithium manganese oxide (LMO) |
| Fluorinated Ester 2 | lithium and manganese-rich cathode (LMR or LLMO) |
| Fluorinated Ester 2 | lithium iron phosphate (LFP) |
| Fluorinated Ester 2 | lithium cobalt phosphate (LCP) |
| Fluorinated Ester 2 | lithium manganese phosphate (LMP) |
| Fluorinated Ester 2 | lithium manganese iron phosphate (LMFP) |
| Fluorinated Ester 2 | transition metal sulfide |
| Fluorinated Ester 2 | sulfur |
| Fluorinated Ester 2 | sulfur/carbon composite |
| Fluorinated Ester 3 | lithium nickel manganese cobalt oxide (NMC) |
| Fluorinated Ester 3 | lithium nickel cobalt aluminum oxide (NCA) |
| Fluorinated Ester 3 | lithium nickel manganese aluminum oxide (NMA) |
| Fluorinated Ester 3 | lithium nickel manganese cobalt aluminum oxide (NMCA) |
| Fluorinated Ester 3 | lithium nickel oxide (LNO) |
| Fluorinated Ester 3 | lithium nickel manganese oxide (LiNi_{0.5}Mn_{1.5}O₄) |
| Fluorinated Ester 3 | lithium cobalt oxide (LCO) |
| Fluorinated Ester 3 | lithium manganese oxide (LMO) |
| Fluorinated Ester 3 | lithium and manganese-rich cathode (LMR or LLMO) |
| Fluorinated Ester 3 | lithium iron phosphate (LFP) |
| Fluorinated Ester 3 | lithium cobalt phosphate (LCP) |
| Fluorinated Ester 3 | lithium manganese phosphate (LMP) |
| Fluorinated Ester 3 | lithium manganese iron phosphate (LMFP) |
| Fluorinated Ester 3 | transition metal sulfide |
| Fluorinated Ester 3 | sulfur |
| Fluorinated Ester 3 | sulfur/carbon composite |

TABLE 13

| Fluorinated Ester | Cathode Material |
|---|---|
| Fluorinated Ester 1 | sodium vanadium phosphate |
| Fluorinated Ester 1 | sodium copper nickel iron manganese oxide |
| Fluorinated Ester 1 | Prussian white |
| Fluorinated Ester 1 | Prussian blue |
| Fluorinated Ester 1 | sodium iron manganese oxide |
| Fluorinated Ester 1 | sodium nickel manganese oxide |
| Fluorinated Ester 1 | sodium ferric phosphate pyrophosphate |
| Fluorinated Ester 1 | sodium vanadium phosphate pyrophosphate |
| Fluorinated Ester 1 | sodium ferric sulfate |
| Fluorinated Ester 1 | sodium manganese oxide |
| Fluorinated Ester 1 | sodium manganese phosphate |
| Fluorinated Ester 1 | sodium nickel manganese iron oxide |
| Fluorinated Ester 1 | sodium iron nickel oxide |
| Fluorinated Ester 1 | sodium iron phosphate |
| Fluorinated Ester 1 | sodium iron fluorophosphate |
| Fluorinated Ester 1 | sodium iron manganese fluorophosphate |
| Fluorinated Ester 1 | sodium vanadium fluorophosphate |
| Fluorinated Ester 1 | sodium iron pyrophosphate |
| Fluorinated Ester 1 | sodium manganese pyrophosphate |
| Fluorinated Ester 1 | sodium vanadium pyrophosphate |
| Fluorinated Ester 1 | transition metal sulfide |
| Fluorinated Ester 1 | sulfur |
| Fluorinated Ester 1 | sulfur/carbon composite |
| Fluorinated Ester 2 | sodium vanadium phosphate |
| Fluorinated Ester 2 | sodium copper nickel iron manganese oxide |
| Fluorinated Ester 2 | Prussian white |
| Fluorinated Ester 2 | Prussian blue |
| Fluorinated Ester 2 | sodium iron manganese oxide |
| Fluorinated Ester 2 | sodium nickel manganese oxide |
| Fluorinated Ester 2 | sodium ferric phosphate pyrophosphate |
| Fluorinated Ester 2 | sodium vanadium phosphate pyrophosphate |
| Fluorinated Ester 2 | sodium ferric sulfate |
| Fluorinated Ester 2 | sodium manganese oxide |
| Fluorinated Ester 2 | sodium manganese phosphate |
| Fluorinated Ester 2 | sodium nickel manganese iron oxide |
| Fluorinated Ester 2 | sodium iron nickel oxide |
| Fluorinated Ester 2 | sodium iron phosphate |
| Fluorinated Ester 2 | sodium iron fluorophosphate |
| Fluorinated Ester 2 | sodium iron manganese fluorophosphate |
| Fluorinated Ester 2 | sodium vanadium fluorophosphate |
| Fluorinated Ester 2 | sodium iron pyrophosphate |
| Fluorinated Ester 2 | sodium manganese pyrophosphate |
| Fluorinated Ester 2 | sodium vanadium pyrophosphate |
| Fluorinated Ester 2 | transition metal sulfide |
| Fluorinated Ester 2 | sulfur |
| Fluorinated Ester 2 | sulfur/carbon composite |
| Fluorinated Ester 3 | sodium vanadium phosphate |
| Fluorinated Ester 3 | sodium copper nickel iron manganese oxide |
| Fluorinated Ester 3 | Prussian white |
| Fluorinated Ester 3 | Prussian blue |
| Fluorinated Ester 3 | sodium iron manganese oxide |
| Fluorinated Ester 3 | sodium nickel manganese oxide |
| Fluorinated Ester 3 | sodium ferric phosphate pyrophosphate |
| Fluorinated Ester 3 | sodium vanadium phosphate pyrophosphate |
| Fluorinated Ester 3 | sodium ferric sulfate |
| Fluorinated Ester 3 | sodium manganese oxide |
| Fluorinated Ester 3 | sodium manganese phosphate |
| Fluorinated Ester 3 | sodium nickel manganese iron oxide |
| Fluorinated Ester 3 | sodium iron nickel oxide |
| Fluorinated Ester 3 | sodium iron phosphate |
| Fluorinated Ester 3 | sodium iron fluorophosphate |
| Fluorinated Ester 3 | sodium iron manganese fluorophosphate |
| Fluorinated Ester 3 | sodium vanadium fluorophosphate |
| Fluorinated Ester 3 | sodium iron pyrophosphate |
| Fluorinated Ester 3 | sodium manganese pyrophosphate |
| Fluorinated Ester 3 | sodium vanadium pyrophosphate |
| Fluorinated Ester 3 | transition metal sulfide |
| Fluorinated Ester 3 | sulfur |
| Fluorinated Ester 3 | sulfur/carbon composite |

A battery is formed with an electrolyte from Tables 3-5 (for formation of a lithium-ion battery) or Tables 6-8 (for formation of a sodium-ion battery), optionally including a, e.g., at least one, supplemental solvent in Tables 9A-9F, an anode including an anode material from Table 10 (for formation of a lithium-ion battery) or Table 11 (for formation of a sodium-ion battery), and a cathode including a cathode material from Table 12 (for formation of a lithium-ion battery) or Table 13 (for formation of a sodium-ion battery). Depending on properties of the battery, the battery is prepared in ambient conditions, a dry room, or an argon-filled glove box. The battery is prepared as a coin cell, pouch cell, cylindrical cell, or prismatic cell form factor with a nominal capacity of 10 milliampere-hours (mAh)-1,000 ampere-hours (Ah). Electrolyte is added to each cell.

### VI. Battery Performance

Electrochemical cycling tests is carried out using a battery tester with temperature controlled at −60° C.-60° C. The battery is first subjected to formation cycles at C/100-1 C charge rate and C/100-1 C discharge rate with a lower voltage limit of 0 volts (V)-3 V and upper voltage limit of 1.5 V-6.0 V. As used herein, a C-rate means a current which will discharge a battery in one hour, e.g., a C-rate for a battery having a discharge capacity of 1.6 ampere-hours would be 1.6 amperes.

The battery is subjected to extended cycling using a C/100-1 C charge rate and C/100-1 C discharge rate. Electrochemical performance parameters of the battery include the number of cycles to reach 80% of initial capacity and the battery resistance growth from electrochemical impedance spectroscopy (EIS) measurements. Batteries with the electrolyte formulation exhibit a higher initial (dis)charge capacity, higher number of cycles before reaching 80% capacity retention, reduced impedance, or a combination thereof compared to a comparative example.

### EXAMPLES

### Synthetic Methods:

Provided herein are methods of preparing fluorinated esters, as illustrated by the following examples.

### Synthetic Example 1A: Synthesis of Fluorinated Ester 1 (1,2-difluoroethyl 3,3,3-trifluoropropanoate)

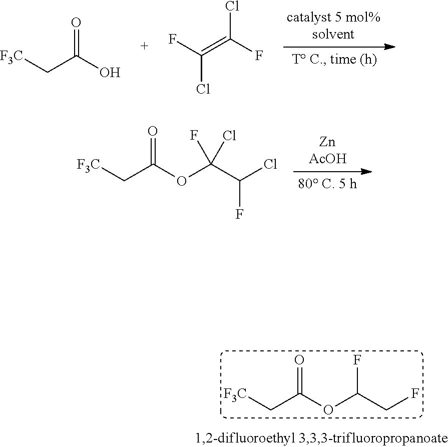

### Example 1A, Step 1

In a typical reaction employing a fluorinated olefin, an autoclave is used. In a nitrogen glovebox the autoclave chamber is loaded with 5 mol % of a suitable catalyst selected from Iron(III) trifluoromethanesulfonate (Fe(OTf)₃), Aluminum (III) trifluoromethanesulfonate (Al(OTf)₃), Scandium trifluoromethanesulfonate (Sc(OTf)₃), Silver trifluoromethanesulfonate (AgOTf), Copper trifluoromethanesulfonate (Cu(OTf)₂, (triphenylphosphine)goldtrifluoromethanesulfonate ((PPh₃)AuOTf), N-bromosuccinimide (NBS), iodine (I₂), or trifluoromethanesulfonic acid (TfOH). The chamber is sealed, brought outside the glove box and trifluoropropanoic acid (0.05 moles to 0.5 moles (mol), 1-10 equivalents (equiv.)) is added. A solvent selected from acetonitrile (MeCN), 1,4-dioxane, tetrahydrofuran (THF), diglyme, benzonitrile (PhCN), or toluene, can be added or the reaction conducted solvent free (250 mL). The autoclave is equipped with a thermocouple and pressure gauge, cooled down with a dry ice/acetone bath and evacuated. Then, (E)-1,2-dichloro-1,2-difluoroethene (0.05 mol to 0.5 mol, 1-10 equiv.) is transferred into the autoclave under vacuum. The resulting mixture is warmed to room temperature and then heated at 50° C.-120° C. with stirring for 2-24 hours (h). After cooling to room temperature, the reaction mix is transferred to a suitable vessel and purified by distillation. The resulting chloro-fluoroester can then be subjected to reductive dechlorination conditions to afford the fluorinated product.

### Example 1A, Step 2: Reductive Dechlorination with Zinc

Inside a nitrogen glovebox, zinc powder (3 equiv.) is added to a round bottom flask equipped with a stir bar. The flask is sealed and brought outside the glovebox. Next, acetic acid (3 equiv.) is added via syringe, and the corresponding ester is added next, dropwise with a syringe. The resulting suspension is heated to 80° C. and stirred for 5 h. After cooling to room temperature, the mixture is quenched with saturated NaHCO₃ solution at 0° C. and extracted with ether three times. The organic phase is dried with MgSO₄ and distilled to obtain the target compound.

### Synthetic Example 1B: Synthesis of Fluorinated Ester 2 (1,1-difluoroethyl 3,3,3-trifluoropropanoate)

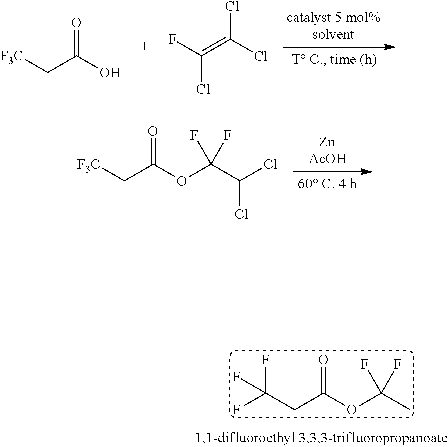

### Example 1B, Step 1

In a typical reaction employing a fluorinated olefin, an autoclave is used. In a nitrogen glovebox the autoclave chamber is loaded with 5 mol % of a suitable catalyst selected from Iron(III) trifluoromethanesulfonate (Fe(OTf)₃), Aluminum (III) trifluoromethanesulfonate (Al(OTf)₃), Scandium trifluoromethanesulfonate (Sc(OTf)₃), Silver trifluoromethanesulfonate (AgOTf), Copper trifluoromethanesulfonate (Cu(OTf)₂, (triphenylphosphine)goldtrifluoromethanesulfonate ((PPh₃)AuOTf), N-bromosuccinimide (NBS), iodine (I₂), or trifluoromethanesulfonic acid (TfOH). The chamber is sealed, brought outside the glove box and trifluoropropanoic acid (0.05 moles to 0.5 moles (mol), 1-10 equivalents (equiv.)) is added. A solvent selected from acetonitrile (MeCN), 1,4-dioxane, tetrahydrofuran (THF), diglyme, benzonitrile (PhCN), or toluene, can be added or the reaction conducted solvent free (250 mL). The autoclave is equipped with a thermocouple and pressure gauge, cooled down with a dry ice/acetone bath and evacuated. Then, 1,1-dichloro-2,2-difluoroethene (0.05 mol to 0.5 mol, 1-10 equiv.) is transferred into the autoclave under vacuum. The resulting mixture is warmed to room temperature and then heated at 50° C.-120° C. with stirring for 2-24 hours (h). After cooling to room temperature, the reaction mix is transferred to a suitable vessel and purified by distillation. The resulting chloro-fluoroester can then be subjected to reductive dechlorination conditions to afford the fluorinated product.

### Example 1B, Step 2: Reductive Dechlorination with Zinc

Inside a nitrogen glovebox, zinc powder (3 equiv.) is added to a round bottom flask equipped with a stir bar. The flask is sealed and brought outside the glovebox. Next, acetic acid (3 equiv.) is added via syringe, and the corresponding ester is added next, dropwise with a syringe. The resulting suspension is heated to 60° C. and stirred for 4 h. After cooling to room temperature, the mixture is quenched with saturated NaHCO₃ solution at 0° C. and extracted with ether three times. The organic phase is dried with MgSO₄ and distilled to obtain the target compound.

### Synthetic Example 1 C: Synthesis of Fluorinated Ester 3 (1,1-difluoroethyl 2,2-difluoroacetate)

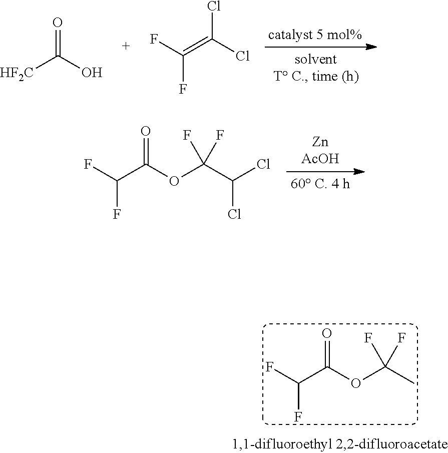

### Example 1 C, Step 1

In a typical reaction employing a fluorinated olefin, an autoclave is used. In a nitrogen glovebox the autoclave chamber is loaded with 5 mol % of a suitable catalyst selected from Iron(III) trifluoromethanesulfonate (Fe(OTf)₃), Aluminum (III) trifluoromethanesulfonate (Al(OTf)₃), Scandium trifluoromethanesulfonate (Sc(OTf)₃), Silver trifluoromethanesulfonate (AgOTf), Copper trifluoromethanesulfonate (Cu(OTf)₂, (triphenylphosphine)goldtrifluoromethanesulfonate ((PPh₃)AuOTf), N-bromosuccinimide (NBS), iodine (I₂), or trifluoromethanesulfonic acid (TfOH). The chamber is sealed, brought outside the glove box and difluoroacetic acid (0.05 moles to 0.5 moles (mol), 1-10 equivalents (equiv.)) is added. A solvent selected from acetonitrile (MeCN), 1,4-dioxane, tetrahydrofuran (THF), diglyme, benzonitrile (PhCN), or toluene, can be added or the reaction conducted solvent free (250 mL). The autoclave is equipped with a thermocouple and pressure gauge, cooled down with a dry ice/acetone bath and evacuated. Then, 1,1-dichloro-2,2-difluoroethene (0.05 mol to 0.5 mol, 1-10 equiv.) is transferred into the autoclave under vacuum. The resulting mixture is warmed to room temperature and then heated at 50° C.-120° C. with stirring for 2-24 hours (h). After cooling to room temperature, the reaction mix is transferred to a suitable vessel and purified by distillation. The resulting chloro-fluoroester can then be subjected to reductive dechlorination conditions to afford the fluorinated product.

### Example 1 C, Step 2: Reductive Dechlorination with Zinc

Inside a nitrogen glovebox, zinc powder (3 equiv.) is added to a round bottom flask equipped with a stir bar. The flask is sealed and brought outside the glovebox. Next, acetic acid is added via syringe, and the corresponding ester is added next, dropwise with a syringe. The resulting suspension is heated to 60° C. and stirred for 4 h. After cooling to room temperature, the mixture is quenched with saturated NaHCO₃ solution at 0° C. and extracted with ether three times. The organic phase is dried with MgSO₄ and distilled to obtain the target compound.

### ALTERNATIVE Synthetic Route Using Sodium Carboxylate Salts:

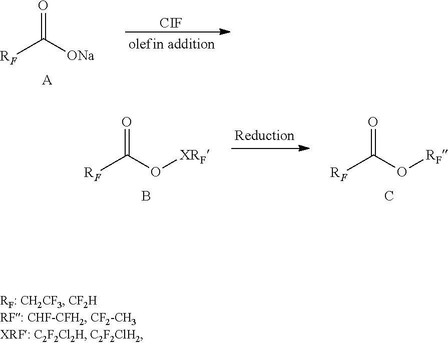

### Synthetic example 2A: Synthesis of Fluorinated Ester 1 (1,2-difluoroethyl 3,3,3-trifluoropropanoate)

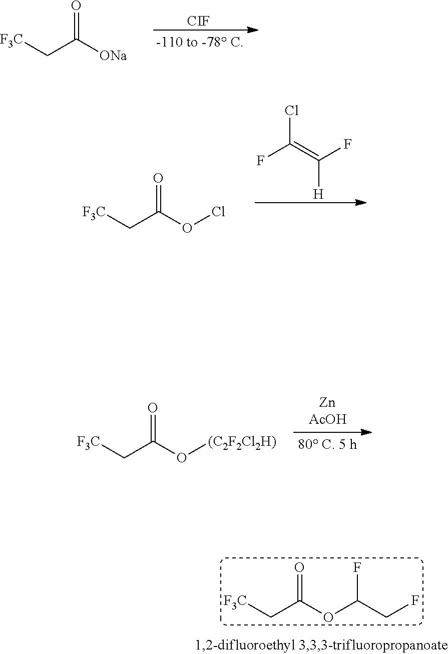

### Example 2A, Step 1

To prepare the hypochlorous anhydride, 0.5 mol of dried sodium 3,3,3-trifluoropropanoate are added to a passivated Hastelloy autoclave equipped with a thermocouple and a pressure gauge. The reactor is then evacuated and cooled to −195° C. Then, CIF (0.15 mol) is carefully added to the autoclave at this temperature. The mixture is warmed to −110° C. and stirred at this temperature for 5 hours (h). When the internal temperature reaches −78° C., this temperature is then maintained for 12 h. After this time, the reactor headspace is evacuated to remove any unreacted CIF and volatile by products that are collected into a cylinder cooled to −110° C. Then, the autoclave is further cooled to −195° C. and 1,2-difluoro-1-chloroethylene (0.15 mol) is added under vacuum. The mixture is stirred for 24 hours, allowing the reactor to slowly warm up over time. The resulting ester is then collected, distilled and used for the next step.

### Synthetic Example 2A: Step 2

Inside a nitrogen glovebox, zinc powder (3 equiv.) is added to a round bottom flask equipped with a stir bar. The flask is sealed and brought outside the glovebox. Next, acetic acid (3 equiv.) is added via syringe, and the corresponding ester is added next, dropwise with a syringe. The resulting suspension is heated to 80° C. and stirred for 5 h. After cooling to room temperature, the mixture is quenched with saturated NaHCO₃ solution at 0° C. and extracted with ether three times. The organic phase is dried with MgSO₄ and distilled to obtain the target compound.

### Synthetic Example 2B: Synthesis of Fluorinated Ester 2 (1,1-difluoroethyl 3,3,3-trifluoropropanoate)

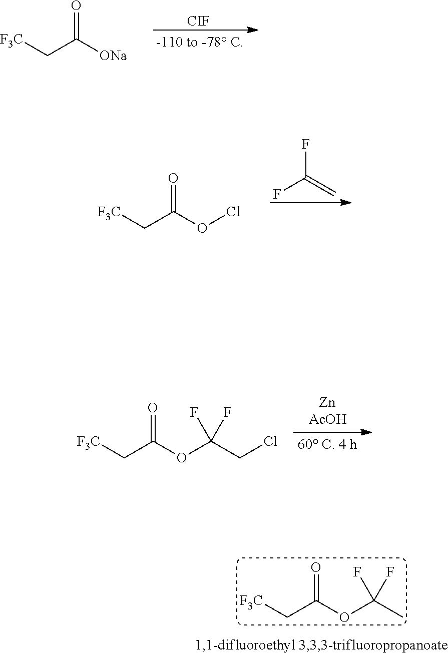

### Example 2B, Step 1

To prepare the hypochlorous anhydride, 0.5 mol of dried sodium 3,3,3-trifluoropropanoate are added to a passivated Hastelloy autoclave equipped with a thermocouple and a pressure gauge. The reactor is then evacuated and cooled to −195° C. Then, CIF (0.15 mol) is carefully added to the autoclave at this temperature. The mixture is warmed to −110° C. and stirred at this temperature for 5 hours. When the internal temperature reaches −78° C., this temperature is then maintained for 12 h. After this time, the reactor headspace is evacuated to remove any unreacted CIF and volatile by products that are collected into a cylinder cooled to −110° C. Then, the autoclave is further cooled to −195° C. and 1,1-difluoroethylene (0.15 mol) is added under vacuum. The mixture is stirred for 24 hours, allowing the reactor to slowly warm up over time. The resulting ester is then collected, distilled and used for the next step.

### Synthetic Example 2B, Step 2: Reductive Dechlorination with Zinc

Inside a nitrogen glovebox, zinc powder (3 equiv.) is added to a round bottom flask equipped with a stir bar. The flask is sealed and brought outside the glovebox. Next, acetic acid (3 equiv.) is added via syringe, and the corresponding ester is added next, dropwise with a syringe. The resulting suspension is heated to 80° C. and stirred for 5 h. After cooling to room temperature, the mixture is quenched with saturated NaHCO₃ solution at 0° C. and extracted with ether three times. The organic phase is dried with MgSO₄ and distilled to obtain the target compound.

### Synthetic Example 2C: Synthesis of Fluorinated Ester 3 (1,1-difluoroethyl 2,2-difluoroacetate)

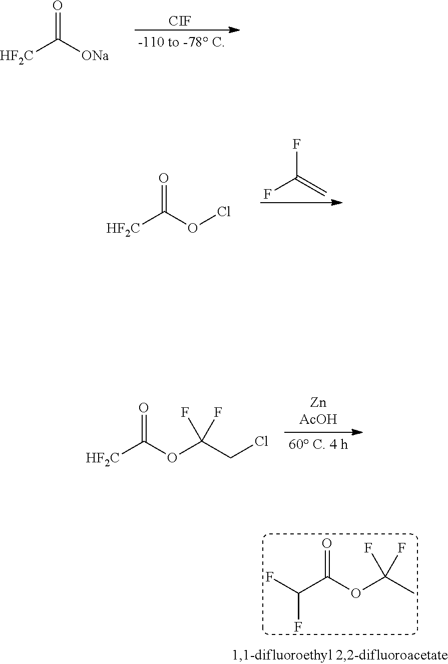

### Synthetic Example 2C, Step 1

To prepare the hypochlorous anhydride, 0.5 mol of dried sodium 2,2-difluoroacetate are added to a passivated Hastelloy autoclave equipped with a thermocouple and a pressure gauge. The reactor is then evacuated and cooled to −195° C. Then, CIF (0.15 mol) is carefully added to the autoclave at this temperature. The mixture is warmed to −110° C. and stirred at this temperature for 5 hours. When the internal temperature reaches −78° C., this temperature is then maintained for 12 h. After this time, the reactor headspace is evacuated to remove any unreacted CIF and volatile by products that are collected into a cylinder cooled to −110° C. Then, the autoclave is further cooled to −195° C. and 1,1-difluoroethylene (0.15 mol) is added under vacuum. The mixture is stirred for 24 hours, allowing the reactor to slowly warm up over time. The resulting ester is then collected, distilled and used for the next step.

### Synthetic Example 2C, Step 2: Reductive Dechlorination with Zinc

Inside a nitrogen glovebox, zinc powder (3 equiv.) is added to a round bottom flask equipped with a stir bar. The flask is sealed and brought outside the glovebox. Next, acetic acid (3 equiv.) is added via syringe, and the corresponding ester is added next, dropwise with a syringe. The resulting suspension is heated to 80° C. and stirred for 5 h. After cooling to room temperature, the mixture is quenched with saturated NaHCO₃ solution at 0° C. and extracted with ether three times. The organic phase is dried with MgSO₄ and distilled to obtain the target compound.

### Example 3A (Lithium-Ion Electrolyte Formulation)

An electrolyte including 1,2-difluoroethyl 3,3,3-trifluoropropanoate (Fluorinated Ester 1) is prepared by dissolving lithium hexafluorophosphate (LiPF₆) (Lithium Salt 4) at 1 moles per liter (molar, M) concentration in a solvent mixture of 1,2-difluoroethyl 3,3,3-trifluoropropanoate (Fluorinated Ester 1) and fluoroethylene carbonate (FEC). The amount of 1,2-difluoroethyl 3,3,3-trifluoropropanoate in the solvent mixture is 90 weight percent (wt. %) and the amount of fluoroethylene carbonate in the solvent mixture is 10 wt. %, based on a total weight of the solvent mixture. Further examples of electrolyte formulation include substitution of Fluorinated Ester 1 with at least one of Fluorinated Esters 2 and 3, addition to Fluorinated Ester 1 of at least one of Fluorinated Esters 2 and 3, substitution of Lithium Salt 4 with at least one of Lithium Salts 1-3 or 5-16, addition to Lithium Salt 4 of at least one of Lithium Salts 1-3 or 5-16 (see Tables 3-5), omission of FEC, substitution of FEC with a different supplemental solvent in Tables 9A-9F, addition to FEC of a different supplemental solvent in Tables 9A-9F, or a combination thereof.

### Example 3B (Lithium-Ion Battery Formation)

The electrolyte of Example 3A is tested in a battery with a graphite anode, a lithium nickel manganese cobalt oxide (NMC) cathode, and a polypropylene separator. The battery is prepared in an argon-filled glove box and has a pouch cell form factor with a nominal capacity of 300 milliampere-hours (mAh). An amount of 1 mL of electrolyte is added to each cell. Further examples of battery formation include substitution of the graphite anode with a different anode material in Table 10, substitution of the NMC cathode with a different cathode material in Table 12, or a combination thereof.

### Example 3C (Lithium-Ion Battery Performance)

Electrochemical cycling tests of the battery of Example 3B is carried out using a Maccor, Inc. battery tester with temperature controlled at 40° C. The battery is first subjected to 3 formation cycles at C/20 charge rate and C/20 discharge rate with a lower voltage limit of 3.0 V and upper voltage limit of 4.5 V. After completion of formation cycles, the battery is degassed and re-sealed in the argon-filled glove box.

The battery is subjected to extended cycling using a 0.5 C charge rate and 0.5 C discharge rate. Electrochemical performance parameters of the battery include the number of cycles to reach 80% of initial capacity and the battery resistance growth from electrochemical impedance spectroscopy (EIS) measurements. Batteries with the electrolyte formulation exhibit more cycles before reaching 80% capacity retention compared to batteries utilizing a 1 M LiPF₆ 3:7 ethylene carbonate:dimethyl carbonate electrolyte.

### Example 4A (Lithium-Ion Electrolyte Formulation)

Lithium hexafluorophosphate (Lithium Salt 4) at 1 M concentration is dissolved in a 9:1 volume ratio mixture of at least one of Fluorinated Esters 1-3:fluoroethylene carbonate (FEC). Further examples of electrolyte formulation include substitution of Lithium Salt 4 with at least one of Lithium Salts 1-3 or 5-16, addition to Lithium Salt 4 of at least one of Lithium Salts 1-3 or 5-16 (see Tables 3-5), substitution of FEC with a different supplemental solvent in Tables 9A-9F, addition to FEC of a different supplemental solvent in Tables 9A-9F, or a combination thereof.

### Example 4B (Lithium Ion Battery Formation)

The electrolyte of Example 4A is tested in a battery with a graphite anode, lithium nickel manganese cobalt oxide (NMC) cathode, and polypropylene separator. The battery is prepared in an argon-filled glove box and has a pouch cell form factor with a nominal capacity of 300 mAh. Further examples of battery formation include substitution of the graphite anode with a different anode material in Table 10, substitution of the NMC cathode with a different cathode material in Table 12, or a combination thereof.

### Example 4C (Lithium-Ion Battery Performance)

The electrochemical cycling tests of the battery of Example 4B are carried out using a Maccor Inc. battery tester with temperature control at 40° C. The battery is first subjected to three formation cycles at C/20 rate (charge and discharge over a period of 20 hours). After completion of formation cycles, the battery is degassed and re-sealed in the argon filled glove box. The battery is subjected to extended cycling using a 0.5 C charge rate and 0.5 C discharge rate. Electrochemical performance parameters of the battery include the number of cycles to reach 80% of initial capacity and the battery resistance growth from electrochemical impedance spectroscopy (EIS) measurements. Batteries with the electrolyte formulation exhibit more cycles before reaching 80% capacity retention compared to batteries utilizing a 1 M LiPF₆ 3:7 ethylene carbonate: dimethyl carbonate electrolyte.

### Example 5A (Sodium-Ion Electrolyte Formulation)

An electrolyte including 1,2-difluoroethyl 3,3,3-trifluoropropanoate (Fluorinated Ester 1) is prepared by dissolving sodium hexafluorophosphate (NaPF₆) (Sodium Salt 4) at 1 M concentration in a solvent mixture of 1,2-difluoroethyl 3,3,3-trifluoropropanoate (Fluorinated Ester 1) and fluoroethylene carbonate (FEC). The amount of 1,2-difluoroethyl 3,3,3-trifluoropropanoate in the solvent mixture is 90 weight percent (wt. %) and the amount of fluoroethylene carbonate in the solvent mixture is 10 wt. %, based on a total weight of the solvent mixture. Further examples of electrolyte formulation include substitution of Fluorinated Ester 1 with at least one of Fluorinated Esters 2 and 3, addition to Fluorinated Ester 1 of at least one of Fluorinated Esters 2 and 3, substitution of Sodium Salt 4 with at least one of Sodium Salts 1-3 or 5-13, addition to Sodium Salt 4 of at least one of Sodium Salts 1-3 or 5-13 (see Tables 6-8), omission of FEC, substitution of FEC with a different supplemental solvent in Tables 9A-9F, addition to FEC of a different supplemental solvent in Tables 9A-9F, or a combination thereof.

### Example 5B (Sodium-Ion Battery Formation)

The electrolyte of Example 5A is tested in a battery with a hard carbon anode, a sodium nickel manganese iron oxide cathode, and a polypropylene separator. The battery is prepared in an argon-filled glove box and has a pouch cell form factor with a nominal capacity of 200 milliampere-hours (mAh). An amount of 1 mL of electrolyte is added to each cell. Further examples of battery formation include substitution of the hard carbon anode with a different anode material in Table 11, substitution of the sodium nickel manganese iron oxide cathode with a different cathode material in Table 13, or a combination thereof.

### Example 5C (Sodium-Ion Battery Performance)

Electrochemical cycling tests of the battery of Example 5B is carried out using a Maccor, Inc. battery tester with temperature controlled at 40° C. The battery is first subjected to 3 formation cycles at C/20 charge rate and C/20 discharge rate with a lower voltage limit of 3.0 V and upper voltage limit of 4.0 V. After completion of formation cycles, the battery is degassed and re sealed in the argon filled glove box.

The battery is subjected to extended cycling using a 0.5 C charge rate and 0.5 C discharge rate. Electrochemical performance parameters of the battery include the number of cycles to reach 80% of initial capacity and the battery resistance growth from electrochemical impedance spectroscopy (EIS) measurements. Batteries with the electrolyte formulation exhibit more cycles before reaching 80% capacity retention compared to batteries utilizing a 1 M NaPF₆ 3:7 ethylene carbonate: dimethyl carbonate electrolyte.

### Example 6A (Sodium-Ion Electrolyte Formulation)

Sodium hexafluorophosphate (Sodium Salt 4) at 1 M concentration is dissolved in a 9:1 volume ratio mixture of at least one of Fluorinated Esters 1-3:fluoroethylene carbonate (FEC). Further examples of electrolyte formulation include substitution of Sodium Salt 4 with at least one of Sodium Salts 1-3 or 5-13, addition to Sodium Salt 4 of at least one of Sodium Salts 1-3 or 5-13 (see Tables 6-8), substitution of FEC with a different supplemental solvent in Tables 9A-9F, addition to FEC of a different supplemental solvent in Tables 9A-9F, or a combination thereof.

### Example 6B (Sodium-Ion Battery Formation)

The electrolyte of Example 6A is tested in a battery with a hard carbon anode, sodium nickel manganese iron oxide cathode, and polypropylene separator. The battery is prepared in an argon-filled glove box and has a pouch cell form factor with a nominal capacity of 300 mAh. Further examples of battery formation include substitution of the hard carbon anode with a different anode material in Table 11, substitution of the sodium nickel manganese iron oxide with a different cathode material in Table 13, or a combination thereof.

### Example 6C (Sodium-Ion Battery Performance)

The electrochemical cycling tests of the battery of Example 6B are carried out using a Maccor Inc. battery tester with temperature control at 40° C. The battery is first subjected to three formation cycles at C/20 rate (charge and discharge over a period of 20 hours). After completion of formation cycles, the battery is degassed and re sealed in the argon filled glove box. The battery is then subjected to extended cycling using a 0.5 C charge rate and 0.5 C discharge rate. Electrochemical performance parameters of the battery include the number of cycles to reach 80% of initial capacity and the battery resistance growth from electrochemical impedance spectroscopy (EIS) measurements. Batteries with the electrolyte formulation exhibit more cycles before reaching 80% capacity retention compared to batteries utilizing a 1 M NaPF₆ 3:7 v:v ethylene carbonate: dimethyl carbonate electrolyte.

### ASPECTS

This disclosure further encompasses the following aspects.
Aspect 1: A fluorinated ester selected from 1,2-difluoroethyl 3,3,3-trifluoropropanoate, 1,1-difluoroethyl 3,3,3-trifluoropropanoate, or 1,1-difluoroethyl 2,2-difluoroacetate.
Aspect 2: A method of synthesizing a fluorinated ester, the method comprising: providing a carboxylic acid or carboxylic acid derivative; and reacting the carboxylic acid or carboxylic acid derivative with a fluorinated olefin to synthesize the fluorinated ester.
Aspect 3: The method of claim 2, wherein the method comprises: providing a fluorinated carboxylate salt; reacting the fluorinated carboxylate salt with chlorine monofluoride to form a fluorinated hypochlorous anhydride; reacting the fluorinated hypochlorous anhydride with a fluorinated olefin to synthesize a fluorinated ester intermediate; and selectively dehalogenating the fluorinated ester intermediate to synthesize the fluorinated ester.
Aspect 4: The method of aspect 2, wherein the carboxylic acid or carboxylic acid derivative is fluorinated.
Aspect 5: The method of aspect 2, wherein the reaction is in the presence of a phase transfer catalyst or phase transfer agent.
Aspect 6: The method of aspect 2, wherein the method further comprises selectively dehalogenating the fluorinated ester.
Aspect 7: The method of aspect 2, wherein the method further comprises reductively dechlorinating the fluorinated ester.
Aspect 8: The method of aspect 7, wherein reductively dechlorinating the fluorinated ester is promoted by zinc.
Aspect 9: The method of aspect 2, wherein the fluorinated olefin comprises a gem-difluoroolefin, a trihalosubstituted alkene, or a tetrahalosubstituted alkene.
Aspect 10: The method of aspect 2, wherein the fluorinated olefin is (E)-1,2-dichloro-1,2-difluoroethene, (Z)-1,2-dichloro-1,2-difluoroethene, 1,1-difluoroethene, or 1,1-dichloro-2,2-difluoroethene.
Aspect 11: The method of aspect 2, wherein the carboxylic acid comprises 3,3,3-trifluoropropanoic acid, difluoroacetic acid, 3-fluoropropanoic acid, or propanoic acid.
Aspect 12: The method of aspect 2, wherein the carboxylic acid derivative comprises a derivative of 3,3,3-trifluoropropanoic acid, difluoroacetic acid, 3-fluoropropanoic acid, or propanoic acid.
Aspect 13: The method of aspect 2, wherein the carboxylic acid derivative comprises an acid fluoride, a sodium carboxylate salt, or a hypochlorous anhydride.
Aspect 14: A fluorinated ester synthesized by the method of aspect 2, wherein the fluorinated ester is 1,2-difluoroethyl 3,3,3-trifluoropropanoate.
Aspect 15: A fluorinated ester synthesized by the method of aspect 2, wherein the fluorinated ester is 1,1-difluoroethyl 3,3,3-trifluoropropanoate.
Aspect 16: A fluorinated ester synthesized by the method of aspect 2, wherein the fluorinated ester is 1,1-difluoroethyl 2,2-difluoroacetate.
Aspect 17: A method of synthesizing a fluorinated ester, the method comprising: providing a fluorinated carboxylic acid or fluorinated carboxylic acid derivative; reacting the fluorinated carboxylic acid or fluorinated carboxylic acid derivative with a fluorinated olefin to synthesize a fluorinated ester intermediate; and selectively dehalogenating the fluorinated ester intermediate to synthesize the fluorinated ester.
Aspect 18: A fluorinated ester synthesized by the method of aspect 17, wherein the fluorinated ester is 1,2-difluoroethyl 3,3,3-trifluoropropanoate, 1,1-difluoroethyl 3,3,3-trifluoropropanoate, or 1,1-difluoroethyl 2,2-difluoroacetate.
Aspect 19: An electrolyte comprising: 1,2-difluoroethyl 3,3,3-trifluoropropanoate, 1,1-difluoroethyl 3,3,3-trifluoropropanoate, 1,1-difluoroethyl 2,2-difluoroacetate, or a combination thereof; and lithium bis(trifluoromethanesulfonyl)imide, lithium bis(fluorosulfonyl)imide, lithium bis(oxalato)borate, lithium hexafluorophosphate, lithium tetrafluoroborate, lithium perchlorate, lithium hexafluoroarsenate, lithium triflate, lithium nitrate, lithium difluorophosphate, lithium difluoro(oxalato)borate, lithium trifluoroacetate, lithium 4,5-dicyano-2-(trifluoromethyl)imidazole, lithium difluoro(dioxalato)phosphate, lithium tetrafluoro(oxalato)phosphate, lithium bis(pentafluoroethanesulfonyl)imide, or a combination thereof.
Aspect 20: The electrolyte of aspect 19, further comprising fluoroethylene carbonate, propylene carbonate, dimethyl carbonate, diethyl carbonate, ethyl methyl carbonate, vinyl carbonate, vinyl ethylene carbonate, ethylene carbonate, difluoroethylene carbonate, 3,3,3-trifluoropropylene carbonate, monofluoroethyl methyl carbonate, difluoroethyl methyl carbonate, trifluoroethyl methyl carbonate, bis(2,2,2-trifluoroethyl) carbonate, 2,2-difluoroethyl ethyl carbonate, acetonitrile, fluoroacetonitrile, ethyl acetate, methyl acetate, methyl propanoate, methyl 3,3,3-trifluoropropanoate, ethyl 3,3,3-trifluoropropanoate, ethyl 2,2-difluoropropanoate, isopropyl trifluoroacetate, methyl 4,4,4-trifluorobutanoate, methyl difluoroacetate, 2,2,2-trifluoroethyl butanoate, methyl 2,2-difluoropropanoate, ethyl trifluoroacetate, 2,2-difluoroethyl trifluoroacetate, 2,2,2-trifluoroethyl acetate, ethyl 2,2-difluoroacetate, 2,2-difluoroethyl acetate, 2,2-difluoroethyl 3,3-difluoropropanoate, 2,2-difluoroethyl 2,2-difluoroacetate, succinonitrile, propionitrile, butyronitrile, adiponitrile, 1,3,6-hexanetricarbonitrile, trimethyl borate, triphenyl borate, triethyl borate, tris(pentafluorophenyl)borane, tris(trimethylsilyl)phosphate, tris(2,2,2-trifluoroethyl) borate, trimethyl phosphate, triethyl phosphate, tris(trimethylsilyl)phosphate, tris(trimethylsilyl)phosphite, tris(2,2,2-trifluoroethyl) phosphate, tris(2,2,2-trifluoroethyl) phosphite, (pentafluorophenyl)diphenyl phosphine, tris(pentafluorophenyl) phosphine, 1,3,2-dioxathiolane-2,2-dioxide, 1,3-propanesultone, prop-1-ene-1,3-sultone, propanediol cyclic sulfate, 1,3,2-dioxathiolane 2-oxide, bis(2,2,2-trifluoroethyl) ether, 1,1,2,2-tetrafluoroethyl-2,2,3,3-tetrafluoropropyl ether, 1H,1H,5H-octafluoropentyl-1,1,2,2-tetrafluoroethylether, 2,2,2-trifluoroethyl 1,1,2,2-tetrafluoroethyl ether, tetrahydrofuran, 1,3-dioxolane, 1,2-dimethoxyethane, diethylene glycol dimethyl ether, tetraethylene glycol dimethyl ether, succinic anhydride, butyric anhydride, 1,4-dioxane, tetravinyl silane, 1,4-butane sultone, dimethyl sulfoxide, methylene methanedisulfonate, N,N-dimethylformamide, gamma-butyrolactone, or a combination thereof.
Aspect 21: A battery comprising: the electrolyte of aspect 19 or 20; an anode comprising graphite, lithium metal, silicon, a silicon oxide, a graphite/silicon composite, a graphite/silicon oxide composite, a silicon/carbon composite, a silicon carbide, a graphite/silicon carbide composite, a silicon nitride, a graphite/silicon nitride composite, a hard carbon, an amorphous carbon, or lithium titanium oxide; and a cathode comprising lithium nickel manganese cobalt oxide, lithium nickel cobalt aluminum oxide, lithium nickel manganese aluminum oxide, lithium nickel manganese cobalt aluminum oxide, lithium nickel oxide, lithium nickel manganese oxide, lithium cobalt oxide, lithium manganese oxide, lithium and manganese-rich cathode, lithium iron phosphate, lithium cobalt phosphate, lithium manganese phosphate, lithium manganese iron phosphate, a transition metal sulfide, sulfur, or a sulfur/carbon composite.
Aspect 22: An electrolyte comprising: 1,2-difluoroethyl 3,3,3-trifluoropropanoate, 1,1-difluoroethyl 3,3,3-trifluoropropanoate, 1,1-difluoroethyl 2,2-difluoroacetate, or a combination thereof; and sodium bis(trifluoromethanesulfonyl)imide, sodium bis(fluorosulfonyl)imide, sodium bis(oxalato)borate, sodium hexafluorophosphate, sodium tetrafluoroborate, sodium perchlorate, sodium hexafluoroarsenate, sodium triflate, sodium nitrate, sodium difluorophosphate, sodium difluoro(oxalato)borate, sodium trifluoroacetate, sodium 4,5-dicyano-2-(trifluoromethyl)imidazole, or a combination thereof.
Aspect 23: The electrolyte of aspect 22, further comprising fluoroethylene carbonate, propylene carbonate, dimethyl carbonate, diethyl carbonate, ethyl methyl carbonate, vinyl carbonate, vinyl ethylene carbonate, ethylene carbonate, difluoroethylene carbonate, 3,3,3-trifluoropropylene carbonate, monofluoroethyl methyl carbonate, difluoroethyl methyl carbonate, trifluoroethyl methyl carbonate, bis(2,2,2-trifluoroethyl) carbonate, 2,2-difluoroethyl ethyl carbonate, acetonitrile, fluoroacetonitrile, ethyl acetate, methyl acetate, methyl propanoate, methyl 3,3,3-trifluoropropanoate, ethyl 3,3,3-trifluoropropanoate, ethyl 2,2-difluoropropanoate, isopropyl trifluoroacetate, methyl 4,4,4-trifluorobutanoate, methyl difluoroacetate, 2,2,2-trifluoroethyl butanoate, methyl 2,2-difluoropropanoate, ethyl trifluoroacetate, 2,2-difluoroethyl trifluoroacetate, 2,2,2-trifluoroethyl acetate, ethyl 2,2-difluoroacetate, 2,2-difluoroethyl acetate, 2,2-difluoroethyl 3,3-difluoropropanoate, 2,2-difluoroethyl 2,2-difluoroacetate, succinonitrile, propionitrile, butyronitrile, adiponitrile, 1,3,6-hexanetricarbonitrile, trimethyl borate, triphenyl borate, triethyl borate, tris(pentafluorophenyl)borane, tris(trimethylsilyl)phosphate, tris(2,2,2-trifluoroethyl) borate, trimethyl phosphate, triethyl phosphate, tris(trimethylsilyl)phosphate, tris(trimethylsilyl)phosphite, tris(2,2,2-trifluoroethyl) phosphate, tris(2,2,2-trifluoroethyl) phosphite, (pentafluorophenyl)diphenyl phosphine, tris(pentafluorophenyl) phosphine, 1,3,2-dioxathiolane-2,2-dioxide, 1,3-propanesultone, prop-1-ene-1,3-sultone, propanediol cyclic sulfate, 1,3,2-dioxathiolane 2-oxide, bis(2,2,2-trifluoroethyl) ether, 1,1,2,2-tetrafluoroethyl-2,2,3,3-tetrafluoropropyl ether, 1H,1H,5H-octafluoropentyl-1,1,2,2-tetrafluoroethylether, 2,2,2-trifluoroethyl 1,1,2,2-tetrafluoroethyl ether, tetrahydrofuran, 1,3-dioxolane, 1,2-dimethoxyethane, diethylene glycol dimethyl ether, tetraethylene glycol dimethyl ether, succinic anhydride, butyric anhydride, 1,4-dioxane, tetravinyl silane, 1,4-butane sultone, dimethyl sulfoxide, methylene methanedisulfonate, N,N-dimethylformamide, gamma-butyrolactone, or a combination thereof.
Aspect 24: A battery comprising: the electrolyte of aspect 22 or 23; an anode comprising sodium metal, graphite, silicon, a silicon oxide, a graphite/silicon composite, a graphite/silicon oxide composite, a silicon/carbon composite, a silicon carbide, a graphite/silicon carbide composite, a silicon nitride, a graphite/silicon nitride composite, a hard carbon, an amorphous carbon, sodium titanium oxide, tin, a tin oxide, or a tin sulfide; and a cathode comprising sodium vanadium phosphate, sodium copper nickel iron, manganese oxide, Prussian white, Prussian blue, sodium iron manganese oxide, sodium nickel manganese oxide, sodium ferric phosphate pyrophosphate, sodium vanadium phosphate pyrophosphate, sodium ferric sulfate, sodium manganese oxide, sodium manganese phosphate, sodium nickel manganese iron oxide, sodium iron nickel oxide, sodium iron phosphate, sodium iron fluorophosphate, sodium iron manganese fluorophosphate, sodium vanadium fluorophosphate, sodium iron pyrophosphate, sodium manganese pyrophosphate, sodium vanadium pyrophosphate, a transition metal sulfide, sulfur, or a sulfur/carbon composite.
Aspect 25: An electrolyte comprising: 1,2-difluoroethyl 3,3,3-trifluoropropanoate, 1,1-difluoroethyl 3,3,3-trifluoropropanoate, 1,1-difluoroethyl 2,2-difluoroacetate, or a combination thereof; fluoroethylene carbonate, propylene carbonate, dimethyl carbonate, diethyl carbonate, ethyl methyl carbonate, vinyl carbonate, vinyl ethylene carbonate, ethylene carbonate, difluoroethylene carbonate, 3,3,3-trifluoropropylene carbonate, monofluoroethyl methyl carbonate, difluoroethyl methyl carbonate, trifluoroethyl methyl carbonate, bis(2,2,2-trifluoroethyl) carbonate, 2,2-difluoroethyl ethyl carbonate, acetonitrile, fluoroacetonitrile, ethyl acetate, methyl acetate, methyl propanoate, methyl 3,3,3-trifluoropropanoate, ethyl 3,3,3-trifluoropropanoate, ethyl 2,2-difluoropropanoate, isopropyl trifluoroacetate, methyl 4,4,4-trifluorobutanoate, methyl difluoroacetate, 2,2,2-trifluoroethyl butanoate, methyl 2,2-difluoropropanoate, ethyl trifluoroacetate, 2,2-difluoroethyl trifluoroacetate, 2,2,2-trifluoroethyl acetate, ethyl 2,2-difluoroacetate, 2,2-difluoroethyl acetate, 2,2-difluoroethyl 3,3-difluoropropanoate, 2,2-difluoroethyl 2,2-difluoroacetate, succinonitrile, propionitrile, butyronitrile, adiponitrile, 1,3,6-hexanetricarbonitrile, trimethyl borate, triphenyl borate, triethyl borate, tris(pentafluorophenyl)borane, tris(trimethylsilyl)phosphate, tris(2,2,2-trifluoroethyl) borate, trimethyl phosphate, triethyl phosphate, tris(trimethylsilyl)phosphate, tris(trimethylsilyl)phosphite, tris(2,2,2-trifluoroethyl) phosphate, tris(2,2,2-trifluoroethyl) phosphite, (pentafluorophenyl)diphenyl phosphine, tris(pentafluorophenyl) phosphine, 1,3,2-dioxathiolane-2,2-dioxide, 1,3-propanesultone, prop-1-ene-1,3-sultone, propanediol cyclic sulfate, 1,3,2-dioxathiolane 2-oxide, bis(2,2,2-trifluoroethyl) ether, 1,1,2,2-tetrafluoroethyl-2,2,3,3-tetrafluoropropyl ether, 1H,1H,5H-octafluoropentyl-1,1,2,2-tetrafluoroethylether, 2,2,2-trifluoroethyl 1,1,2,2-tetrafluoroethyl ether, tetrahydrofuran, 1,3-dioxolane, 1,2-dimethoxyethane, diethylene glycol dimethyl ether, tetraethylene glycol dimethyl ether, succinic anhydride, butyric anhydride, 1,4-dioxane, tetravinyl silane, 1,4-butane sultone, dimethyl sulfoxide, methylene methanedisulfonate, N,N-dimethylformamide, gamma-butyrolactone, or a combination thereof; and a metal salt.
Aspect 26: An electrolyte comprising: 1,2-difluoroethyl 3,3,3-trifluoropropanoate, 1,1-difluoroethyl 3,3,3-trifluoropropanoate, 1,1-difluoroethyl 2,2-difluoroacetate, or a combination thereof; and a metal salt.
Aspect 27: The electrolyte of aspect 26, where the metal salt comprises lithium bis(trifluoromethanesulfonyl)imide, lithium bis(fluorosulfonyl)imide, lithium bis(oxalato)borate, lithium hexafluorophosphate, lithium tetrafluoroborate, lithium perchlorate, lithium hexafluoroarsenate, lithium triflate, lithium nitrate, lithium difluorophosphate, lithium difluoro(oxalato)borate, lithium trifluoroacetate, lithium 4,5-dicyano-2-(trifluoromethyl)imidazole, lithium difluoro(dioxalato)phosphate, lithium tetrafluoro(oxalato)phosphate, lithium bis(pentafluoroethanesulfonyl)imide, or a combination thereof.
Aspect 28: The electrolyte of aspect 26, where the metal salt comprises sodium bis(trifluoromethanesulfonyl)imide, sodium bis(fluorosulfonyl)imide, sodium bis(oxalato)borate, sodium hexafluorophosphate, sodium tetrafluoroborate, sodium perchlorate, sodium hexafluoroarsenate, sodium triflate, sodium nitrate, sodium difluorophosphate, sodium difluoro(oxalato)borate, sodium trifluoroacetate, sodium 4,5-dicyano-2-(trifluoromethyl)imidazole, or a combination thereof.
Aspect 29: The electrolyte of any one of aspects 26 to 28, further comprising fluoroethylene carbonate, propylene carbonate, dimethyl carbonate, diethyl carbonate, ethyl methyl carbonate, vinyl carbonate, vinyl ethylene carbonate, ethylene carbonate, difluoroethylene carbonate, 3,3,3-trifluoropropylene carbonate, monofluoroethyl methyl carbonate, difluoroethyl methyl carbonate, trifluoroethyl methyl carbonate, bis(2,2,2-trifluoroethyl) carbonate, 2,2-difluoroethyl ethyl carbonate, acetonitrile, fluoroacetonitrile, ethyl acetate, methyl acetate, methyl propanoate, methyl 3,3,3-trifluoropropanoate, ethyl 3,3,3-trifluoropropanoate, ethyl 2,2-difluoropropanoate, isopropyl trifluoroacetate, methyl 4,4,4-trifluorobutanoate, methyl difluoroacetate, 2,2,2-trifluoroethyl butanoate, methyl 2,2-difluoropropanoate, ethyl trifluoroacetate, 2,2-difluoroethyl trifluoroacetate, 2,2,2-trifluoroethyl acetate, ethyl 2,2-difluoroacetate, 2,2-difluoroethyl acetate, 2,2-difluoroethyl 3,3-difluoropropanoate, 2,2-difluoroethyl 2,2-difluoroacetate, succinonitrile, propionitrile, butyronitrile, adiponitrile, 1,3,6-hexanetricarbonitrile, trimethyl borate, triphenyl borate, triethyl borate, tris(pentafluorophenyl)borane, tris(trimethylsilyl)phosphate, tris(2,2,2-trifluoroethyl) borate, trimethyl phosphate, triethyl phosphate, tris(trimethylsilyl)phosphate, tris(trimethylsilyl)phosphite, tris(2,2,2-trifluoroethyl) phosphate, tris(2,2,2-trifluoroethyl) phosphite, (pentafluorophenyl)diphenyl phosphine, tris(pentafluorophenyl) phosphine, 1,3,2-dioxathiolane-2,2-dioxide, 1,3-propanesultone, prop-1-ene-1,3-sultone, propanediol cyclic sulfate, 1,3,2-dioxathiolane 2-oxide, bis(2,2,2-trifluoroethyl) ether, 1,1,2,2-tetrafluoroethyl-2,2,3,3-tetrafluoropropyl ether, 1H,1H,5H-octafluoropentyl-1,1,2,2-tetrafluoroethylether, 2,2,2-trifluoroethyl 1,1,2,2-tetrafluoroethyl ether, tetrahydrofuran, 1,3-dioxolane, 1,2-dimethoxyethane, diethylene glycol dimethyl ether, tetraethylene glycol dimethyl ether, succinic anhydride, butyric anhydride, 1,4-dioxane, tetravinyl silane, 1,4-butane sultone, dimethyl sulfoxide, methylene methanedisulfonate, N,N-dimethylformamide, gamma-butyrolactone, or a combination thereof.
Aspect 30: A battery comprising: the electrolyte of any one of aspects 26 to 29; an anode; a separator; and a cathode, wherein the anode comprises sodium metal, graphite, silicon, a silicon oxide, a graphite/silicon composite, a graphite/silicon oxide composite, a silicon/carbon composite, a silicon carbide, a graphite/silicon carbide composite, a silicon nitride, a graphite/silicon nitride composite, a hard carbon, an amorphous carbon, sodium titanium oxide, tin, a tin oxide, or a tin sulfide; and the cathode comprises sodium vanadium phosphate, sodium copper nickel iron, manganese oxide, Prussian white, Prussian blue, sodium iron manganese oxide, sodium nickel manganese oxide, sodium ferric phosphate pyrophosphate, sodium vanadium phosphate pyrophosphate, sodium ferric sulfate, sodium manganese oxide, sodium manganese phosphate, sodium nickel manganese iron oxide, sodium iron nickel oxide, sodium iron phosphate, sodium iron fluorophosphate, sodium iron manganese fluorophosphate, sodium vanadium fluorophosphate, sodium iron pyrophosphate, sodium manganese pyrophosphate, sodium vanadium pyrophosphate, a transition metal sulfide, sulfur, or a sulfur/carbon composite, or the anode comprises graphite, lithium metal, silicon, a silicon oxide, a graphite/silicon composite, a graphite/silicon oxide composite, a silicon/carbon composite, a silicon carbide, a graphite/silicon carbide composite, a silicon nitride, a graphite/silicon nitride composite, a hard carbon, an amorphous carbon, or lithium titanium oxide; and the cathode comprises lithium nickel manganese cobalt oxide, lithium nickel cobalt aluminum oxide, lithium nickel manganese aluminum oxide, lithium nickel manganese cobalt aluminum oxide, lithium nickel oxide, lithium nickel manganese oxide, lithium cobalt oxide, lithium manganese oxide, lithium and manganese-rich cathode, lithium iron phosphate, lithium cobalt phosphate, lithium manganese phosphate, lithium manganese iron phosphate, transition metal sulfide, sulfur, or a sulfur/carbon composite.

It should be understood that the foregoing description is only illustrative of the present disclosure. Various alternatives and modifications may be devised by those skilled in the art without departing from the disclosure. Accordingly, the present disclosure is intended to embrace all such alternatives, modifications and variances that fall within the scope of the appended claims.

1. A fluorinated ester selected from 1,2-difluoroethyl 3,3,3-trifluoropropanoate, 1,1-difluoroethyl 3,3,3-trifluoropropanoate, or 1,1-difluoroethyl 2,2-difluoroacetate.

2. A method of synthesizing a fluorinated ester, the method comprising:
(a) providing a carboxylic acid or carboxylic acid derivative; and
(b) reacting the carboxylic acid or carboxylic acid derivative with a fluorinated olefin to synthesize the fluorinated ester.

3. The method of claim 2, wherein the method comprises:
(a) providing a fluorinated carboxylate salt;
(b) reacting the fluorinated carboxylate salt with chlorine monofluoride to form a fluorinated hypochlorous anhydride;
(c) reacting the fluorinated hypochlorous anhydride with a fluorinated olefin to synthesize a fluorinated ester intermediate; and
(d) selectively dehalogenating the fluorinated ester intermediate to synthesize the fluorinated ester.

4. The method of claim 2, wherein the method further comprises selectively dehalogenating the fluorinated ester.

5. The method of claim 2, wherein the fluorinated olefin is (E)-1,2-dichloro-1,2-difluoroethene, (Z)-1,2-dichloro-1,2-difluoroethene, 1,1-difluoroethene, or 1,1-dichloro-2,2-difluoroethene.

6. The method of claim 2, wherein the carboxylic acid comprises 3,3,3-trifluoropropanoic acid, difluoroacetic acid, 3-fluoropropanoic acid, or propanoic acid.

7. The method of claim 2, wherein the carboxylic acid derivative comprises a derivative of 3,3,3-trifluoropropanoic acid, or difluoroacetic acid.

8. The method of claim 2, wherein the carboxylic acid derivative comprises a sodium carboxylate salt or a hypochlorous anhydride.

9. A fluorinated ester synthesized by the method of claim 2, wherein the fluorinated ester is 1,2-difluoroethyl 3,3,3-trifluoropropanoate.

10. A fluorinated ester synthesized by the method of claim 2, wherein the fluorinated ester is 1,1-difluoroethyl 3,3,3-trifluoropropanoate.

11. A fluorinated ester synthesized by the method of claim 2, wherein the fluorinated ester is 1,1-difluoroethyl 2,2-difluoroacetate.

12. An electrolyte comprising:
1,2-difluoroethyl 3,3,3-trifluoropropanoate, 1,1-difluoroethyl 3,3,3-trifluoropropanoate, 1,1-difluoroethyl 2,2-difluoroacetate, or a combination thereof; and
a metal salt.

13. The electrolyte of claim 12, wherein the metal salt comprises lithium bis(trifluoromethanesulfonyl)imide, lithium bis(fluorosulfonyl)imide, lithium bis(oxalato)borate, lithium hexafluorophosphate, lithium tetrafluoroborate, lithium perchlorate, lithium hexafluoroarsenate, lithium triflate, lithium nitrate, lithium difluorophosphate, lithium difluoro(oxalato)borate, lithium trifluoroacetate, lithium 4,5-dicyano-2-(trifluoromethyl)imidazole, lithium difluoro(dioxalato)phosphate, lithium tetrafluoro(oxalato)phosphate, lithium bis(pentafluoroethanesulfonyl)imide, or a combination thereof.

14. The electrolyte of claim 12, wherein the metal salt comprises sodium bis(trifluoromethanesulfonyl)imide, sodium bis(fluorosulfonyl)imide, sodium bis(oxalato)borate, sodium hexafluorophosphate, sodium tetrafluoroborate, sodium perchlorate, sodium hexafluoroarsenate, sodium triflate, sodium nitrate, sodium difluorophosphate, sodium difluoro(oxalato)borate, sodium trifluoroacetate, sodium 4,5-dicyano-2-(trifluoromethyl)imidazole, or a combination thereof.

15. The electrolyte of claim 12, further comprising fluoroethylene carbonate, propylene carbonate, dimethyl carbonate, diethyl carbonate, ethyl methyl carbonate, vinyl carbonate, vinyl ethylene carbonate, ethylene carbonate, difluoroethylene carbonate, 3,3,3-trifluoropropylene carbonate, monofluoroethyl methyl carbonate, difluoroethyl methyl carbonate, trifluoroethyl methyl carbonate, bis(2,2,2-trifluoroethyl) carbonate, 2,2-difluoroethyl ethyl carbonate, acetonitrile, fluoroacetonitrile, ethyl acetate, methyl acetate, methyl propanoate, methyl 3,3,3-trifluoropropanoate, ethyl 3,3,3-trifluoropropanoate, ethyl 2,2-difluoropropanoate, isopropyl trifluoroacetate, methyl 4,4,4-trifluorobutanoate, methyl difluoroacetate, 2,2,2-trifluoroethyl butanoate, methyl 2,2-difluoropropanoate, ethyl trifluoroacetate, 2,2-difluoroethyl trifluoroacetate, 2,2,2-trifluoroethyl acetate, ethyl 2,2-difluoroacetate, 2,2-difluoroethyl acetate, 2,2-difluoroethyl 3,3-difluoropropanoate, 2,2-difluoroethyl 2,2-difluoroacetate, succinonitrile, propionitrile, butyronitrile, adiponitrile, 1,3,6-hexanetricarbonitrile, trimethyl borate, triphenyl borate, triethyl borate, tris(pentafluorophenyl)borane, tris(trimethylsilyl)phosphate, tris(2,2,2-trifluoroethyl) borate, trimethyl phosphate, triethyl phosphate, tris(trimethylsilyl)phosphate, tris(trimethylsilyl)phosphite, tris(2,2,2-trifluoroethyl) phosphate, tris(2,2,2-trifluoroethyl) phosphite, (pentafluorophenyl)diphenyl phosphine, tris(pentafluorophenyl) phosphine, 1,3,2-dioxathiolane-2,2-dioxide, 1,3-propanesultone, prop-1-ene-1,3-sultone, propanediol cyclic sulfate, 1,3,2-dioxathiolane 2-oxide, bis(2,2,2-trifluoroethyl) ether, 1,1,2,2-tetrafluoroethyl-2,2,3,3-tetrafluoropropyl ether, 1H,1H,5H-octafluoropentyl-1,1,2,2-tetrafluoroethylether, 2,2,2-trifluoroethyl 1,1,2,2-tetrafluoroethyl ether, tetrahydrofuran, 1,3-dioxolane, 1,2-dimethoxyethane, diethylene glycol dimethyl ether, tetraethylene glycol dimethyl ether, succinic anhydride, butyric anhydride, 1,4-dioxane, tetravinyl silane, 1,4-butane sultone, dimethyl sulfoxide, methylene methanedisulfonate, N,N-dimethylformamide, gamma-butyrolactone, or a combination thereof.
